(19) United States
(12) Patent Application Publication
(10) Pub. No.: US 2010/0029710 A1
(43) Pub. Date: Feb. 4, 2010

(54) COMPOUNDS HAVING BOTH ANGIOTENSIN II RECEPTOR ANTAGONISM AND PPARy ACTIVATING ACTIVITIES

(75) Inventors: Christopher Franklin Bigge, Ann Arbor, MI (US); Agustin Casimiro-Garcia, Mystic, CT (US); Chitase Lee, Ann Arbor, MI (US); Hud Lawrence Risley, Baltic, CT (US); Robert Philip Schaum, Old Saybrook, CT (US)

Correspondence Address: PFIZER INC.;PATENT DEPARTMENT, Bld 114 M/S 114, EASTERN POINT ROAD, GROTON, CT, 06340, US

(73) Assignee: Pfizer Products Inc., Groton, CT (US)

(21) Appl. No.: 12/518,472

(22) PCT Filed: Dec. 3, 2007
(86) PCT No.: PCT/IB2007/003844

§ 371 Date: Jun. 10, 2009

Related U.S. Application Data
(60) Provisional application No. 60/876,334, filed on Dec. 21, 2006.
(60) Provisional application No. 60/970,653, filed on Sep. 7, 2007.

(51) Int. Cl. A61K 31/437; C07D 471/04; A61P 3/10; A61P 3/00; A61P 9/12

(52) U.S. Cl. 514/303; 546/118

(57) ABSTRACT

Compounds of following formula (I) are provided that have both angiotensin II receptor antagonist activity and PPARy agonist activity. Also provided are pharmaceutical compositions comprising the compounds and methods of treatment of diseases with the compounds including type 2 diabetes, insulin resistance, hyperinsulinemia, hyperlipidemia, hypertriglyceridemia, metabolic syndrome, congestive heart failure, and hypertension.

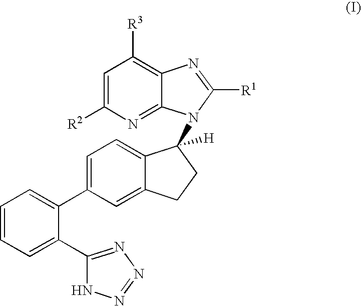

### BACKGROUND OF THE INVENTION

This invention relates to compounds that have both angiotensin II receptor antagonism and PPARy activating activities.

U.S. Pat. No. 5,338,740 and Carpino et al., Bioorganic & Medicinal Chemistry Letters, (1994), Vol. 4, No. 1, 93-8 disclose that certain substituted [2-(1H-tetrazol-5-yl)-phenyl]-indan-1-yl}-3H-imidazo[4,5-b]pyridines are angiotensin II receptor antagonists (ARB). The compounds are useful in the treatment of hypertension, glaucoma, renal disease, congestive heart failure, cognitive dysfunction, and other conditions in which the action of angiotensin II is implicated.

US Publication 2003/0158090 discloses a method of treating diabetes, which comprises administration of an inhibitor of the angiotensin II system and an anti-diabetic agent.

WO 2004/017896 discloses a method of treating hypertension and type-2 diabetes mellitus, metabolic syndrome or pre-diabetic condition comprising administering a combination of a dual PPARα/γ (peroxisome proliferator-activated receptor α/γ) agonist and an angiotensin II type I receptor antagonist. A dual PPARα/γ agonist has both PPARα and PPARγ activity in comparison to the glitazones that have only PPARγ activity.

WO 2004/014308 and US Publication 2004/0127443 disclose a method of treatment and compounds that are angiotensin II type I receptor antagonists (ARB) and can also increase the activity of PPAR's. The diseases that these molecules are used to treat are type-2 diabetes, metabolic syndrome, insulin resistance, and inflammatory disorders.

Peroxisome proliferator-activated receptors (PPARs) are members of the nuclear receptor superfamily of ligand-activated transcription factors. Three subtypes of PPARs have been cloned from the mouse and human: i.e., PPARα, PPARγ, and PPARδ. The PPARs are important regulators of carbohydrate and lipid metabolism, cell growth and differentiation, phenotype transition, apoptosis, neovascularization, immunoregulation and the inflammatory response. Compounds that activate PPARs are useful for the treatment and prevention of a variety of clinical disorders such as type 2 diabetes, insulin resistance, hyperinsulinemia, hyperlipidemia, hypertriglyceridemia, and metabolic syndrome.

Type 2 diabetes is associated with a wide variety of conditions such as hyperglycemia, insulin resistance, hyperinsulinemia, excess weight, high blood pressure, and dyslipidemia (hypertriglyceridemia and low levels of high density lipoproteins), which can lead to the deposition of plaque in the arteries. This cluster of associated conditions has often been referred to as metabolic syndrome, and is strongly associated with an elevated risk for heart disease.

Examples of known compounds that can activate PPARs include thiazolidinediones (e.g. rosiglitazone, pioglitazone, MK 767 (KRP-297), MCC-555, netoglitazone, balaglitazone, rivoglitazone) that primarily activate PPARγ, or PPARγ and PPARα, and non-thiazolidinediones that can activate any combination of PPARα, PPARγ, and PPARδ are JTT-501, LSN862, DRF 4832, LM 4156, LY 510929, LY 519818, TY 51501, X 334, certain tyrosine-based derivatives such as GW1929, and GW7845, phenylacetic acid-based derivatives, phenoxazine phenyl propanoic acid derivatives such as DRF 2725, DRF 2189, cinammic and dihydrocinammic acid-based derivatives such as tesaglitazar (AZ 242)), and 3-Phenyl-7-propylbenzisoxazoles (Adams A D, et al. Bioorg Med Chem Lett. (2003) 13:931-5), that can activate PPARγ in combination with PPARα or PPARδ or both PPARα and PPARδ.

Molecules with PPARγ agonist activity are used to treat type 2 diabetes and are known to decrease insulin resistance, hyperglycemia, hyperinsulinemia, and hypertriglyceridemia. PPARγ agonists, however, are not used for the treatment of high blood pressure. Molecules with angiotensin II receptor antagonist activity are useful in the treatment of high blood pressure. There is a need for a molecule that has both angiotensin II receptor antagonist and PPARγ agonist activities that can be used to treat conditions such as type 2 diabetes, insulin resistance, hyperinsulinemia, hyperlipidemia, hypertriglyceridemia, metabolic syndrome, congestive heart failure, or hypertension.

There remains a need for pharmaceutical agents that have both angiotensin II receptor antagonism and PPARγ activating activities and are useful in the treatment, prevention or diminution of the manifestations of the maladies described herein.

### SUMMARY OF THE INVENTION

The present invention is directed to a compound of the Formula I

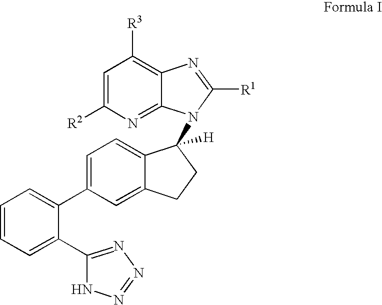

wherein:

R¹ is (C₁-C₄)alkyl or ethoxy;

R² is (C₁-C₈)alkyl, (C₂-C₈)alkenyl or (C₂-C₈)alkynyl, said (C₁-C₈)alkyl, (C₂-C₈)alkenyl or (C₂-C₈)alkynyl mono-, di- or tri-substituted independently with hydroxyl, (C₁-C₅)alkylcarbonyloxy, benzylcarbonyloxy, (C₁-C₃)alkoxy, halo, trifluoromethyl, nitrile, oxo or a 3 to 8 membered partially saturated, fully saturated or fully unsaturated ring optionally having one to three heteroatoms selected independently from one, two or three N, one 0 or one S and said 3 to 8 membered ring optionally mono-, di- or tri-substituted independently with halo, (C₂-C₆)alkenyl, (C₁-C₆)alkyl, hydroxyl, (C₁-C₆)alkoxy, (C₁-C₄)alkylthio, amino, nitro, cyano, oxo, carboxy, (C₁-C₆)alkyloxycarbonyl or mono-N— or di-N,N—(C₁-C₆)alkylamino wherein said (C₁-C₆)alkyl substituent is optionally mono-, di- or tri-substituted independently with halo, hydroxyl, (C₁-C₆)alkoxy, (C₁-C₄)alkylthio, amino, nitro, cyano, oxo, carboxy, (C₁-C₆)alkyloxycarbonyl or mono-N— or di-N,N—(C₁-C₆)alkylamino and wherein said (C₁-C₆)alkyl substituent is also optionally substituted with from one to nine fluorines; and

R³ is CH₃; or

a pharmaceutically acceptable salt thereof.

Another aspect of this invention is directed to methods of treating hypertension, obesity, overweight condition, hypertriglyceridemia, hyperlipidemia, hypoalphalipoproteinemia, Syndrome X (Metabolic Syndrome), diabetes mellitus (especially Type II), hyperinsulinemia, impaired glucose tolerance, insulin resistance, a diabetic complication, atherosclerosis, coronary heart disease, hypercholesterolemia, inflammation, thrombosis or congestive heart failure in a mammal (including a human being) which comprise administering to said mammal a therapeutically effective amount of a compound of Formula I, a prodrug of said compound, or a pharmaceutically acceptable salt of said compound or prodrug.

Also provided herein are compositions comprising a pharmaceutically effective amount of one or more of the compounds described herein and a pharmaceutically acceptable carrier or excipient.

This invention is also directed to pharmaceutical compositions for the treatment of obesity, an overweight condition, hypertriglyceridemia, hyperlipidemia, hypoalphalipoproteinemia, Syndrome X, diabetes mellitus (especially Type II), hyperinsulinemia, impaired glucose tolerance, insulin resistance, a diabetic complication, atherosclerosis, hypertension, coronary heart disease, hypercholesterolemia, inflammation, or congestive heart failure in a mammal (including a human being) which comprise a therapeutically effective amount of a compound of Formula I, a prodrug thereof, or a pharmaceutically acceptable salt of said compound or of said prodrug and a pharmaceutically acceptable vehicle, diluent or carrier.

This invention is also directed to pharmaceutical combination compositions comprising: a therapeutically effective amount of a composition comprising

a first compound, said first compound being a Formula I compound, a prodrug thereof, or a pharmaceutically acceptable salt of said compound or of said prodrug;

a second compound, said second compound being an anti-hypertensive agent; and/or optionally

a pharmaceutical vehicle, diluent or carrier.

Another aspect of this invention is methods for treating hypertension in a mammal comprising administering to a mammal suffering from hypertension

a first compound, said first compound being a Formula I compound a prodrug thereof, or a pharmaceutically acceptable salt of said compound or of said prodrug; and

a second compound, said second compound being an antihypertensive agent wherein the amounts of the first and second compounds result in a therapeutic effect.

Yet another aspect of this invention is kits comprising:

a. a first compound, said first compound being a Formula I compound, a prodrug thereof, or a pharmaceutically acceptable salt of said compound or of said prodrug and a pharmaceutically acceptable carrier, vehicle or diluent in a first unit dosage form;

b. a second compound, said second compound being an anti-hypertensive agent and a pharmaceutically acceptable vehicle, diluent or carrier in a second unit dosage form; and

c. means for containing said first and second dosage forms wherein the amounts of the first and second compounds result in a therapeutic effect.

This invention is also directed to pharmaceutical combination compositions comprising: a therapeutically effective amount of a composition comprising

a first compound, said first compound being a Formula I compound, a prodrug thereof, or a pharmaceutically acceptable salt of said compound or of said prodrug;

a second compound, said second compound being a diabetic treating agent; and/or optionally

a pharmaceutical vehicle, diluent or carrier.

Another aspect of this invention is methods for treating diabetes in a mammal comprising administering to a mammal suffering from diabetes

a first compound, said first compound being a Formula I compound a prodrug thereof, or a pharmaceutically acceptable salt of said compound or of said prodrug; and

a second compound, said second compound being a diabetic treating agent wherein the amounts of the first and second compounds result in a therapeutic effect.

Yet another aspect of this invention is kits comprising:

a. a first compound, said first compound being a Formula I compound, a prodrug thereof, or a pharmaceutically acceptable salt of said compound or of said prodrug and a pharmaceutically acceptable vehicle, diluent or carrier in a first unit dosage form;

b. a second compound, said second compound being a diabetic treating agent and a pharmaceutically acceptable vehicle, diluent or carrier in a second unit dosage form; and

c. means for containing said first and second dosage forms wherein the amounts of the first and second compounds result in a therapeutic effect.

This invention is also directed to pharmaceutical combination composition comprising: a therapeutically effective amount of a composition comprising

a first compound, said first compound being a Formula I compound, a prodrug thereof, or a pharmaceutically acceptable salt of said compound or of said prodrug;

a second compound, said second compound being an antiatherosclerotic agent; and/or optionally

a pharmaceutically acceptable vehicle, diluent or carrier.

Another aspect of this invention is methods for treating atherosclerosis in a mammal comprising administering to a mammal suffering from atherosclerosis

a first compound, said first compound being a Formula I compound, a prodrug thereof, or a pharmaceutically acceptable salt of said compound or of said prodrug; and

a second compound, said second compound being an antiatherosclotic agent wherein the amounts of the first and second compounds result in a therapeutic effect.

Yet another aspect of this invention is kits comprising:

a. a first compound, said first compound being a Formula I compound, a prodrug thereof, or a pharmaceutically acceptable salt of said compound or of said prodrug and a pharmaceutically acceptable carrier, vehicle or diluent in a first unit dosage form;

b. a second compound, said second compound being an antiatherosclerotic agent and a pharmaceutically acceptable carrier, vehicle or diluent in a second unit dosage form; and

c. means for containing said first and second dosage forms wherein the amounts of the first and second compounds result in a therapeutic effect.

This invention is also directed to pharmaceutical combination compositions comprising: a therapeutically effective amount of a composition comprising

a first compound, said first compound being a Formula I compound, a prodrug thereof, or a pharmaceutically acceptable salt of said compound or of said prodrug;

a second compound, said second compound being an anti-obesity agent; and/or optionally

a pharmaceutical vehicle, diluent or carrier.

Another aspect of this invention is methods for treating obesity in a mammal comprising administering to a mammal suffering from obesity

a first compound, said first compound being a Formula I compound a prodrug thereof, or a pharmaceutically acceptable salt of said compound or of said prodrug; and

a second compound, said second compound being an anti-obesity agent wherein the amounts of the first and second compounds result in a therapeutic effect.

Yet another aspect of this invention is kits comprising:

a. a first compound, said first compound being a Formula I compound, a prodrug thereof, or a pharmaceutically acceptable salt of said compound or of said prodrug and a pharmaceutically acceptable carrier, vehicle or diluent in a first unit dosage form;

b. a second compound, said second compound being an anti-obesity agent or a pharmaceutically acceptable vehicle, diluent or carrier in a second unit dosage form; and

c. a container for said first and second dosage forms wherein the amounts of the first and second compounds result in a therapeutic effect.

All patents and patent applications referred to herein are hereby incorporated by reference.

Other features and advantages of this invention will be apparent from this specification and the appendant claims which describe the invention.

### BRIEF DESCRIPTION OF THE DRAWINGS

FIG. 1 is a characteristic x-ray powder diffraction pattern showing that the compound of Example 10 Form A, (S)-1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-2-methylpropan-1-ol, is crystalline. (Vertical Axis: Intensity (CPS); Horizontal Axis: Two theta (degrees))

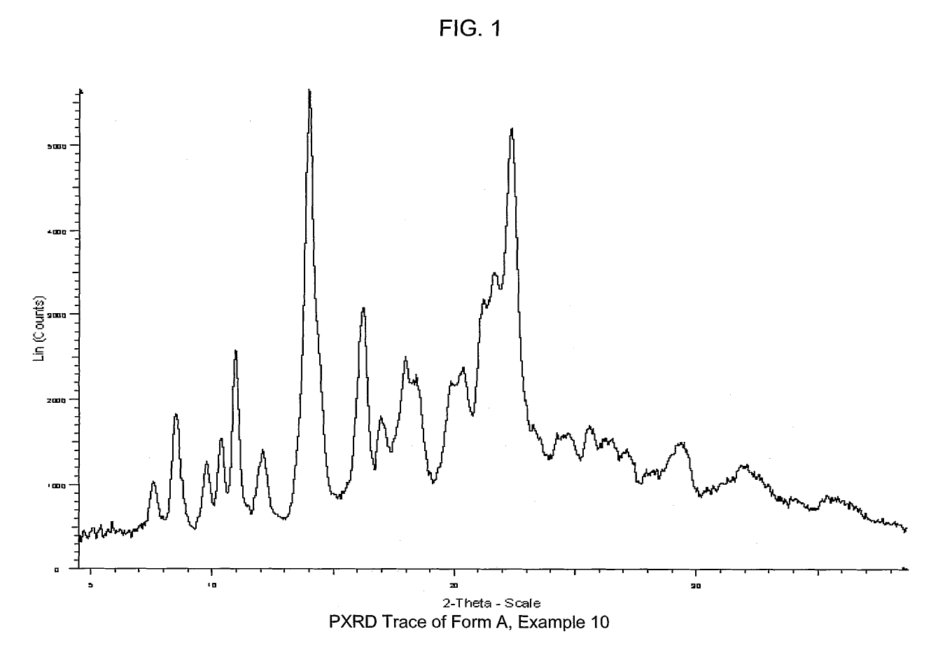

FIG. 2 is a characteristic x-ray powder diffraction pattern showing that the compound of Example 10c Form B, (S)-1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-2-methylpropan-1-ol, is crystalline. (Vertical Axis: Intensity (CPS); Horizontal Axis: Two theta (degrees))

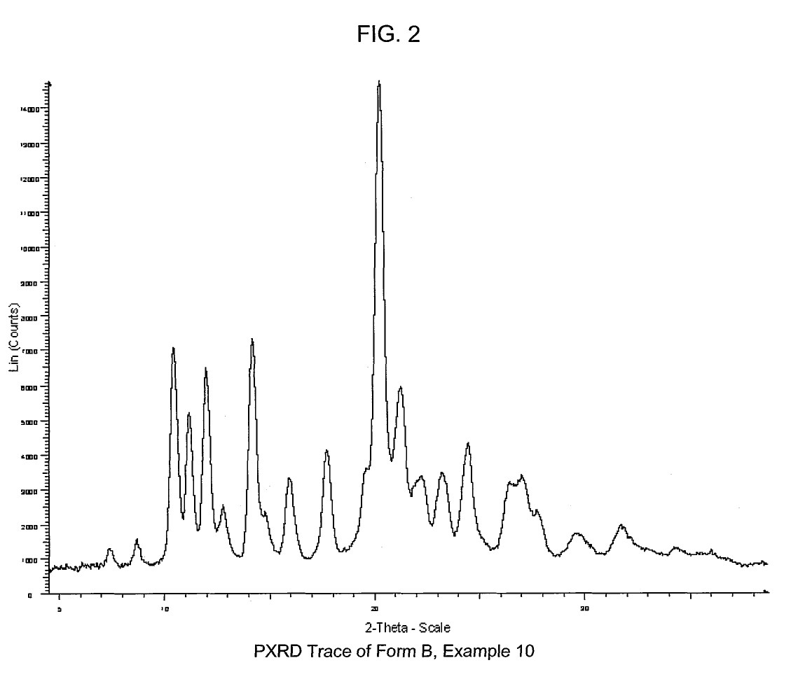

FIG. 3 is the characteristic x-ray powder diffraction pattern of Example 16 Form A of 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-2-methylpropan-2-ol Vertical Axis: Intensity (CPS); Horizontal Axis Two theta (degrees))

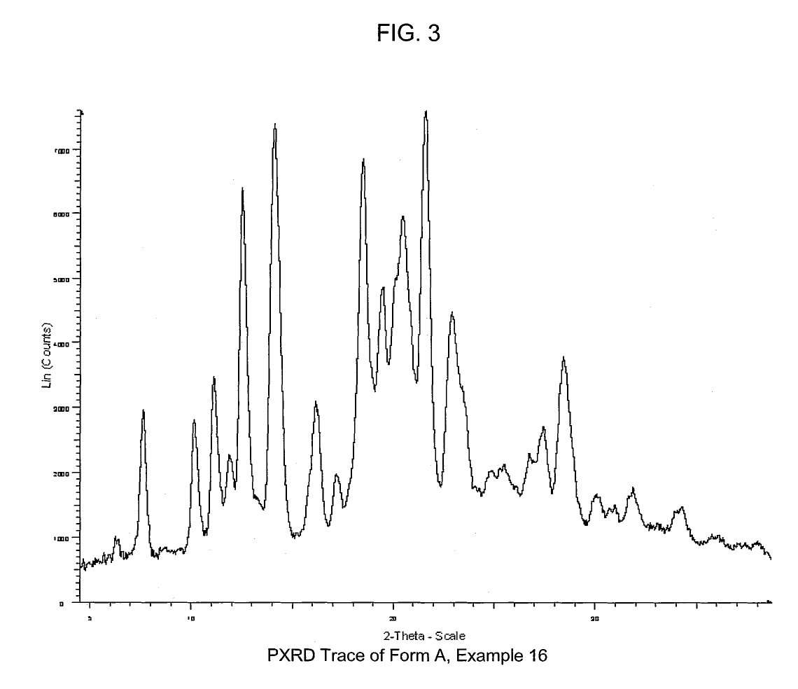

FIG. 4 is the characteristic x-ray powder diffraction pattern of Example 16 Form B 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-2-methylpropan-2-ol Vertical Axis: Intensity (CPS); Horizontal Axis Two theta (degrees))

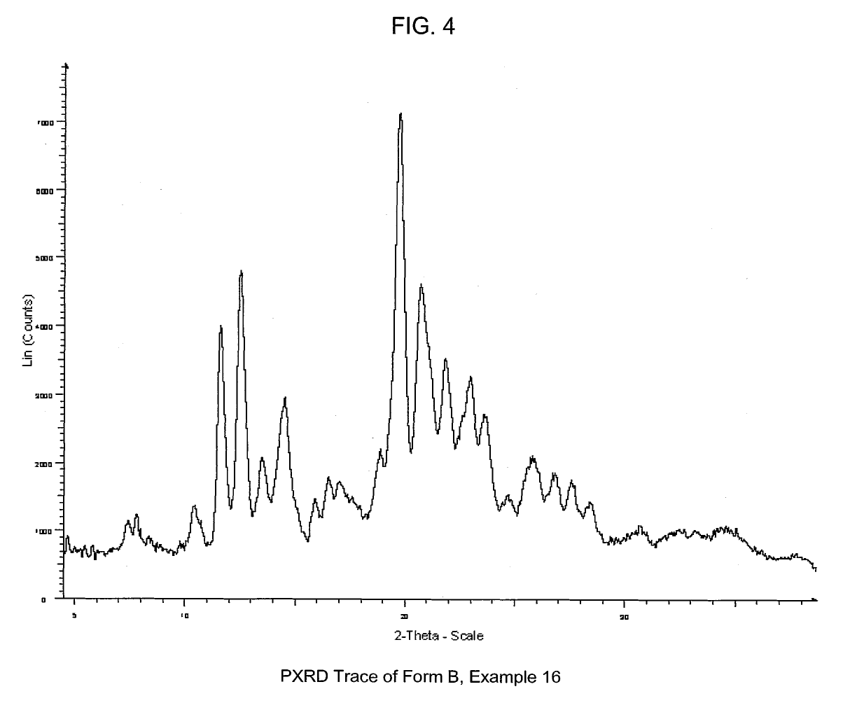

FIG. 5 is the characteristic x-ray powder diffraction pattern of Example 13 Form A, 2-Ethyl-5-(2-methoxy-ethyl)-7-methyl-3-{(S)-5-[2-(1H-tetrazol-5-yl)-phenyl]-indan-1-yl}-3H-imidazo[4,5-b]pyridine, Vertical Axis: Intensity (CPS); Horizontal Axis: Two theta (degrees))

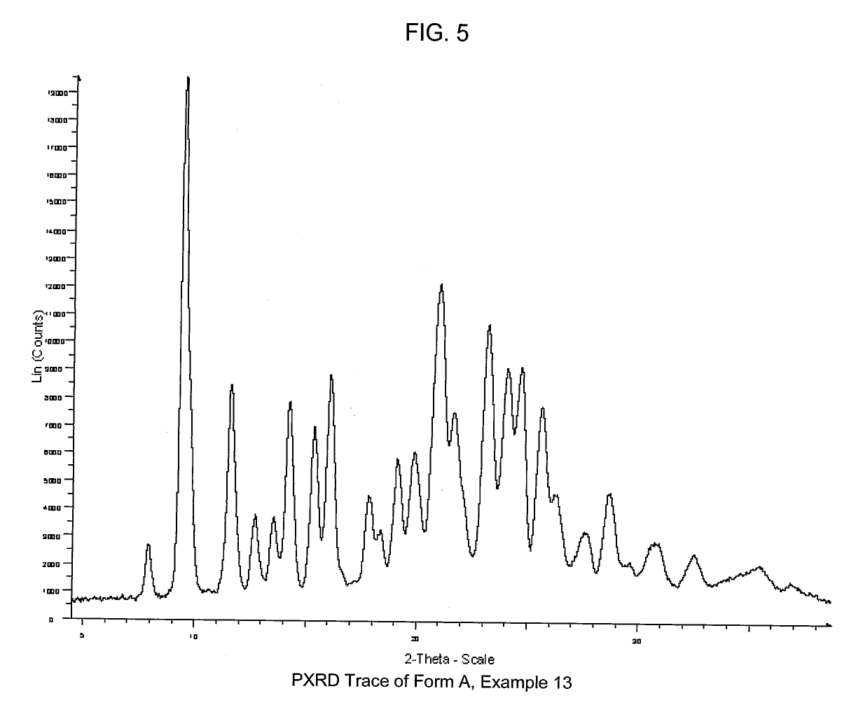

### DETAILED DESCRIPTION OF THE INVENTION

A preferred group of compounds, designated the A Group, contains those compounds having the Formula I as shown above wherein

R¹ is (C₂-C₄) alkyl; and

R² is (C₁-C₈)alkyl, said (C₁-C₈)alkyl mono- or di-substituted independently with hydroxyl, (C₁-C₅)alkylcarbonyloxy, benzylcarbonyloxy, (C₁-C₃)alkoxy, halo, keto or a 5 to 6 membered partially saturated, fully saturated or unsaturated ring optionally having one or two N, and said 5 to 6 membered ring optionally mono-, di- or tri-substituted independently with hydroxy, halo, (C₁-C₃)alkoxy, (C₁-C₄)alkyl or oxo; or

a pharmaceutically acceptable salt thereof.

A group of compounds which is preferred among the A Group of compounds designated the B Group, contains those compounds wherein

R² is (C₂-C₅)alkyl, said (C₂-C₅)alkyl mono-substituted with hydroxyl or (C₁-C₅)alkylcarbonyloxy, benzylcarbonyloxy,

or a pharmaceutically acceptable salt thereof.

A group of compounds which is preferred among the A Group of compounds designated the C Group, contains those compounds wherein

R² is selected from (C₂-C₄)alkyl, said (C₂-C₄)alkyl mono-substituted with (C₁-C₃)alkoxy,

or a pharmaceutically acceptable salt thereof.

A group of compounds which is preferred among the A Group of compounds designated the D Group, contains those compounds wherein

R² is selected from (C₂-C₅)alkyl, said (C₂-C₅)alkyl mono-substituted with a 5 to 6 membered partially saturated, fully saturated or unsaturated ring optionally having one or two N, and said 5 to 6 membered ring optionally mono-, di- or tri-substituted independently with hydroxyl, halo, (C₁-C₃)alkoxy, (C₁-C₄)alkyl or oxo; or

a pharmaceutically acceptable salt thereof.

A group of compounds which is preferred among the A Group of compounds designated the E Group, contains those compounds wherein

R² is selected from (C₂-C₅)alkyl, said (C₂-C₅)alkyl mono-substituted with hydroxyl, (C₁-C₅)alkylcarbonyloxy, benzylcarbonyloxy, or (C₁-C₃)alkoxy, and mono-substituted with a 5 to 6 membered partially saturated, fully saturated or fully unsaturated ring optionally having one or two N, and said 5 to 6 membered ring optionally mono-, di- or tri-substituted independently with hydroxy, halo, (C₁-C₃)alkoxy, (C₁-C₄)alkyl or oxo; or

a pharmaceutically acceptable salt thereof.

A group of compounds which is preferred among the B Group of compounds designated the F Group, contains those compounds wherein

R¹ is ethyl;

R² is (C₂-C₅)alkyl, said (C₂-C₅)alkyl mono-substituted with hydroxyl or (C₁-C₅)alkylcarbonyloxy, benzylcarbonyloxy,

or a pharmaceutically acceptable salt thereof.

A group of compounds which is preferred among the C Group of compounds designated the G Group, contains those compounds wherein

R¹ is ethyl;

R² is selected from (C₂-C₄)alkyl, said (C₂-C₄)alkyl mono-substituted with (C₁-C₃)alkoxy.

Another aspect of this invention is directed to the compounds
a. (S)-1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-2-methylpropan-1-ol);
b. 3-((1S)-5-(2-(1H-tetrazol-5-yl)phenyl)-2,3-dihydro-1H-inden-1-yl)-2-ethyl-5-(2-methoxyethyl)-7-methyl-3H-imidazo[4,5-b]pyridine;
c. 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-2-methylpropan-2-ol;
d. 2-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)ethanol; or
e. 2-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)ethyl acetate

or a pharmaceutically acceptable salt thereof.

An especially preferred compound is

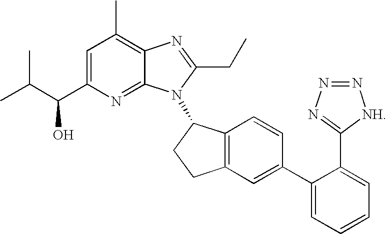

Another aspect of this invention is the compound

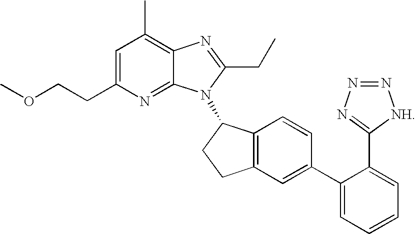

Another aspect of this invention is the compound

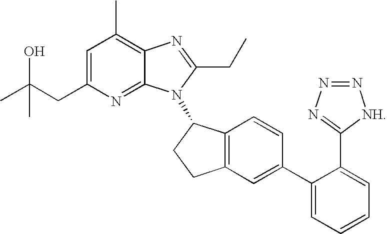

An especially preferred compound is

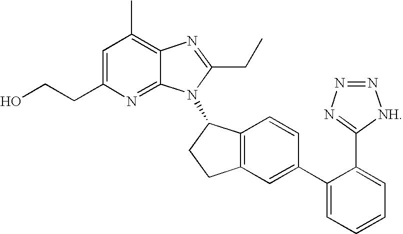

An especially preferred compound is

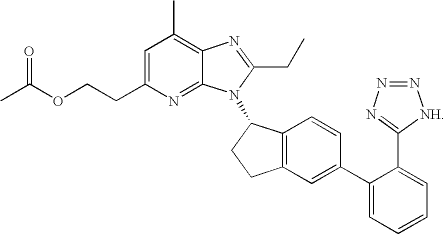

Another aspect of this invention is direct to compounds of Formula IIA

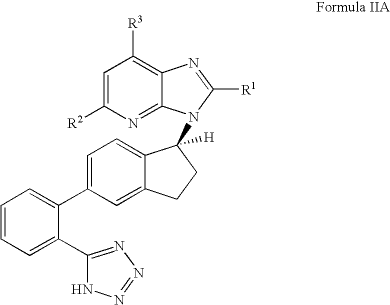

wherein:

R¹ is selected from ethyl, n-propyl, iso-propyl, cyclopropyl, n-butyl, s-butyl, isobutyl, and t-butyl;

R² is n-butyl substituted by 1 or 2 groups selected from OH, C₁-C₃ alkoxy, C(O)OR^{a} or C(O)NR^{a}R^{b} and C₃-C₆ cycloalkyl;

R^{a} is selected from H, C₁-C₆ alkyl, —(CH₂)₀₋₃—(C₃-C₇ cycloalkyl), phenyl and benzyl;

R^{b} is selected from H and C₁-C₆ alkyl; and

R³ is selected from CH₃; or

a pharmaceutically acceptable salt thereof.

Another aspect of this invention is directed to compounds of Formula IIIA:

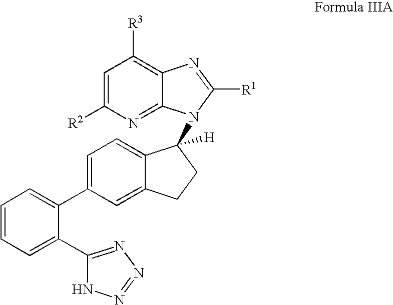

wherein:

R¹ is selected from ethyl, n-propyl iso-propyl, cyclopropyl, n-butyl, s-butyl, isobutyl, and t-butyl;

R² is isobutyl substituted by 1 or 2 groups selected from OH, C₁-C₃ alkoxy, C(O)OR^{a} or C(O)NR^{a}R^{b} and C₃-C₆ cycloalkyl;

R^{a} is selected from H, C₁-C₆ alkyl, —(CH₂)₀₋₃—(C₃-C₇ cycloalkyl), phenyl and benzyl;

R^{b} is selected from H and C₁-C₆ alkyl; and

R³ is CH₃;

or a pharmaceutically acceptable salt thereof.

Another aspect of this invention are the compounds
(S,S)-4-(2-Ethyl-7-methyl-3-{5-[2-(1H-tetrazol-5-yl)-phenyl]-indan-1-yl}-3H-imidazo[4,5-b]pyridin-5-yl)-butan-2-ol;
(S)-1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)propan-2-ol
(R)-1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)propan-2-ol
(S)-1-(2-Ethyl-7-methyl-3-{(S)-5-[2-(1H-tetrazol-5-yl)-phenyl]-indan-1-yl}-3H-imidazo[4,5-b]pyridin-5-yl)-2-methyl-propan-1-ol;
3-((1S)-5-(2-(1H-tetrazol-5-yl)phenyl)-2,3-dihydro-1H-inden-1-yl)-2-ethyl-5-(2-methoxyethyl)-7-methyl-3H-imidazo[4,5-b]pyridine;
1-((S)-2-Ethyl-7-methyl-3-{5-[2-(1H-tetrazol-5-yl)-phenyl]-indan-1-yl}-3H-imidazo[4, 5-]pyridin-5-yl)-2-methyl-propan-2-ol;
3-((1S)-5-(2-(1H-tetrazol-5-yl)phenyl)-2,3-dihydro-1H-inden-1-yl)-2-ethyl-5-(2-methoxypropan-2-yl)-7-methyl-3H-imidazo[4,5-b]pyridine;
3-((1S)-5-(2-(1H-tetrazol-5-yl)phenyl)-2,3-dihydro-1H-inden-1-yl)-2-ethyl-7-methyl-5-(pyridin-2-ylmethyl)-3H-imidazo[4,5-b]pyridine;
(S)-2-Ethyl-7-methyl-5-(2-pyridin-3-yl-ethyl)-3-{5-[2-(1H-tetrazol-5-yl)-phenyl]-indan-1-yl}-3H-imidazo[4,5-b]pyridine;
(S)-2-ethyl-(5-ethyl-[1,3,4]oxadiazol-2-ylmethyl)-7-methyl-3-{5-[2-(1H-tetrazol-5-yl)-phenyl]-indan-1-yl}-3H-imidazo[4,5-b]pyridine;
(S)-(2-Ethyl-7-methyl-3-{5-[2-(1H-tetrazol-5-yl)-phenyl]-indan-1-(S)-yl}-3H-imidazo[4,5-b]pyridin-5-yl)-(S)-phenyl-methanol;
(S)-(2-Ethyl-7-methyl-3-{5-[2-(1H-tetrazol-5-yl)-phenyl]-indan-1-(S)-yl}-3H-imidazo[4,5-b]pyridin-5-yl)-(R)-phenyl-methanol;
2-(S)-(2-Ethyl-7-methyl-3-{5-[2-(1H-tetrazol-5-yl)-phenyl]-indan-1-(S)-yl}-3H-imidazo[4,5-b]pyridin-5-ylmethyl)-cyclohexanone; and
2-(R)-(2-Ethyl-7-methyl-3-{5-[2-(1H-tetrazol-5-yl)-phenyl]-indan-1-(S)-yl}-3H-imidazo[4,5-b]pyridin-5-ylmethyl)-cyclohexanone;
   or a pharmaceutically acceptable salt form thereof.

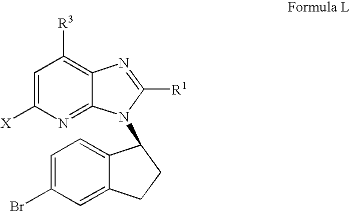

Formula I intermediates include Formula L compounds wherein R¹ is (C₁-C₄)alkyl or ethoxy; X is chloro, bromo, cyano, CH₂OH, CHO, COOMe, (C₁-C₈)alkyl or —(CH₂)ₘ—(C₃-C₆)cycloalkyl; R³ is CH₃; wherein m is 0 or 1; and wherein the cycloalkyl in R¹ and R² may optionally be substituted with 1 methyl group. Examples of useful intermediate compounds of Formula L include:
(S)-3-(5-bromo-2,3-dihydro-1H-inden-1-yl)-5-chloro-2-ethyl-7-methyl-3H-imidazo[4,5-b]pyridine;
(S)-3-(5-bromo-2,3-dihydro-1H-inden-1-yl)-5-bromo-2-ethyl-7-methyl-3H-imidazo[4,5-b]pyridine;
(S)-3-(5-bromo-2,3-dihydro-1H-inden-1-yl)-5-cyano-2-ethyl-7-methyl-3H-imidazo[4,5-b]pyridine;
(S)-3-(5-bromo-2,3-dihydro-1H-inden-1-yl)-5-hydroxymethyl-2-ethyl-7-methyl-3H-imidazo[4,5-b]pyridine;
(S)-methyl 3-(5-bromo-2,3-dihydro-1H-inden-1-yl)-2-ethyl-7-methyl-3H-imidazo[4,5-b]pyridine-5-carboxylate;
(S)-3-(5-bromo-2,3-dihydro-1H-inden-1-yl)-7-methyl-3H-imidazo[4,5-b]pyridine-5-carbaldehyde; and
(S)-5-allyl-3-(5-bromo-2,3-dihydro-1H-inden-1-yl)-2-ethyl-7-methyl-3H-imidazo[4,5-b]pyridine.

Other useful intermediates in the preparation of the compounds of Formula I are compounds of Formula LI and Formula LII.

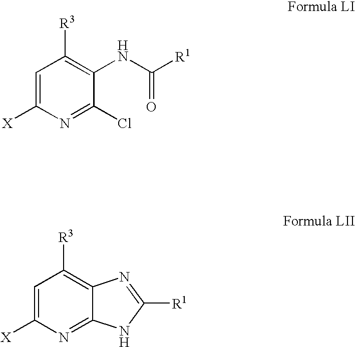

wherein R¹ is (C₁-C₄)alkyl or ethoxy;
X is Cl, Br, allyl, (C₃-C₈)alkyl or —(CH₂)ₘ—(C₃-C₆)cycloalkyl;

### R³ is CH₃;

wherein m is 0 or 1; and wherein the cycloalkyl in R¹ and R² may optionally be substituted with 1 methyl group.
Examples of useful intermediate compounds of Formula LI include
N-(6-allyl-2-chloro-4-methylpyridin-3-yl)propionamide
N-(6-bromo-2-chloro-4-methylpyridin-3-yl)propionamide

Examples of useful intermediates of Formula LII include:
5-chloro-2-ethyl-7-methyl-3H-imidazo[4,5-b]pyridine
5-bromo-2-ethyl-7-methyl-3H-imidazo[4,5-b]pyridine
methyl 2-ethyl-7-methyl-3H-imidazo[4,5-b]pyridine-5-carboxylate
2-ethyl-7-methyl-3H-imidazo[4,5-b]pyridine-5-carbonitrile
5-allyl-2-ethyl-7-methyl-3H-imidazo[4,5-b]pyridine
2-ethyl-5-isobutyl-7-methyl-3H-imidazo[4,5-b]pyridine;
2-ethyl-5-cyclopropylmethyl-7-methyl-3H-imidazo[4,5-b]pyridine;
2-ethyl-5-cyclobutylmethyl-7-methyl-3H-imidazo[4,5-b]pyridine;
2-ethyl-5-cyclopentylmethyl-7-methyl-3H-imidazo[4,5-b]pyridine;
2-ethyl-5-cyclohexylmethyl-7-methyl-3H-imidazo[4,5-b]pyridine;

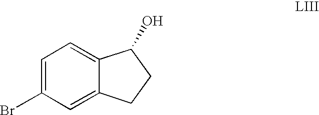

Intermediate LIII is (R)-5-bromo-2,3-dihydro-1H-inden-1-ol

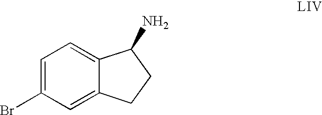

Intermediate LIV is (S)-5-bromo-2,3-dihydro-1H-inden-1-amine

Pharmaceutically acceptable salts of the compounds of Formula I include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002).

The compounds of the invention may exist in both unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water. Pharmaceutically acceptable solvates include hydrates and other solvates wherein the solvent of crystallization may be isotopically substituted, e.g. D₂O, d₆-acetone, d₆-DMSO.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

The compounds of the invention include compounds of Formula I as hereinbefore defined, polymorphs, and isomers thereof (including optical, geometric and tautomeric isomers) as hereinafter defined and isotopically-labelled compounds of Formula I.

The compounds of the present invention may be administered as prodrugs. Thus certain derivatives of compounds of Formula I which may have little or no pharmacological activity themselves can, when administered into or onto the body, be converted into compounds of Formula I having the desired activity, for example, by hydrolytic cleavage. Such derivatives are referred to as ‘prodrugs’. [Further information on the use of prodrugs may be found in Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).]

Prodrugs can, for example, be produced by replacing appropriate functionalities present in the compounds of Formula I with certain moieties known to those skilled in the art as ‘pro-moieties’ as described, for example, in “Design of Prodrugs” by H Bundgaard (Elsevier, 1985).

Some examples of such prodrugs include:

i where the compound of Formula I contains a carboxylic acid functionality (—COOH), an ester thereof, for example, replacement of the hydrogen with (C₁-C₈)alkyl;
ii where the compound of Formula I contains an alcohol functionality (—OH), an ether thereof, for example, replacement of the hydrogen with (C₁-C₆)alkanoyloxymethyl; and
iii where the compound of Formula I contains a primary or secondary amino functionality (—NHH₂ or —NHR where R≠H), an amide thereof, for example, replacement of one or both hydrogens with (C₁-C₁₀)alkanoyl.

In addition, certain compounds of Formula I may themselves act as prodrugs of other compounds of Formula I.

Compounds of Formula I containing an additional asymmetric carbon atom to the 1-indane carbon atom can exist as two or more stereoisomers. Where a compound of Formula I contains an alkenyl or alkenylene group or a cycloalkyl group, geometric cis/trans (or Z/E) isomers are possible. Where the compound contains, for example, a keto or oxime group or an aromatic moiety, tautomeric isomerism (‘tautomerism’) can occur. It follows that a single compound may exhibit more than one type of isomerism.

Included within the scope of the claimed compounds present invention are all stereoisomers, geometric isomers and tautomeric forms of the compounds of Formula (I), including compounds exhibiting more than one type of isomerism, and mixtures of one or more thereof. Also included are acid addition or base salts wherein the counterion is optically active, for example, D-lactate or L-lysine, or racemic, for example, DL-tartrate or DL-arginine.

The present invention includes all pharmaceutically acceptable isotopically-labelled compounds of Formula (I) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of Formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled compounds of Formula (I) can generally be prepared by conventional techniques known to those skilled in the art or by processes analogous to those described in the accompanying Examples and Preparations using an appropriate isotopically-labelled reagents in place of the non-labelled reagent previously employed.

Metabolic Syndrome (Syndrome X) is an increasingly common clinical disorder that refers to an array of risk factors for cardiovascular disease including visceral obesity, insulin resistance, hypertension, disordered glucose metabolism and dyslipidemia. Generally, a patient is deemed to have metabolic syndrome if three of those risk factors are present. Metabolic syndrome greatly increases the likelihood of developing type 2 diabetes and the risk of cardiovascular morbidity and mortality.

References herein to “treatment” include curative, palliative and prophylactic treatment.

As used herein, the expressions “reaction-inert solvent” and “inert solvent” refer to a solvent or a mixture thereof which does not interact with starting materials, reagents, intermediates or products in a manner which adversely affects the yield of the desired product.

By “pharmaceutically acceptable” is meant the carrier, diluent, excipients, and/or salt must be compatible with the other ingredients of the Formulation, and not deleterious to the recipient thereof.

The term “pharmaceutically effective amount”, as used herein, refers to an amount of the compound of Formula I sufficient to treat, prevent onset of or delay or diminish the symptoms and physiological manifestations of the indications described herein.

The term “room temperature” means a temperature between 18 to 25° C., “HPLC” refers to high pressure liquid chromatography, “MPLC” refers to medium pressure liquid chromatography, “TLC” refers to thin layer chromatography, “MS” refers to mass spectrum, “NMR” refers to nuclear magnetic resonance spectroscopy, “DCM” refers to dichloromethane, “DMSO” refers to dimethyl sulfoxide, “DME” refers to dimethoxyethane, “EtOAc” refers to ethyl acetate, “MeOH” refers to methanol, “Ph” refers to the phenyl group, “Pr” refers to propyl, “trityl” refers to the triphenylmethyl group, “ACN” refers to acetonitrile, “DEAD” refers to diethylazodicarboxylate, and “DIAD” refers to diisopropylazodicarboxylate.

Alkyl, alkenyl and alkynyl groups and the alkyl portions of alkoxy groups discussed herein include straight or branched groups having the number of carbon atoms indicated including, for example, methyl, methoxy, ethyl, styrene, propyl, isopropyl, isopropyloxy, allyl, n-butyl, t-butyl, isobutyl, pentyl, isopentyl, and 2-methylbutyl groups. The terms halo or halogen refer to F, Cl, Br or I.

The 3 to 8 membered rings optionally containing independently at least one to three nitrogen ring atoms and optionally having from 1 to 3 additional ring heteroatoms N, one O, or one S include as examples azetidine, oxazetidine, oxazole, isoxazole, oxathiazole, oxadiazolone, isothiazole, thiazole, thiadiazole, imidazole, pyrazole, isopyrazole, 1,3,4-oxadiazole, 1,2,3-oxadiazole, diazole, diazine, oxazine, dioxazine, oxadiazine, thiadiazine, triazole, triazole, tetrazole, oxazine, dioxazine, oxadiazine, thiadiazine, oxathiazole, triazine, thiazine, dithiazine, tetrazine, pentazine, pyrazolidine, tetrazine, triazine, morpholine, thiazine, piperazine, pyrazine, pyridazine, pyrimidine, piperidine and pyridine.

It is to be understood that if a carbocyclic or heterocyclic moiety may be bonded or otherwise attached to a designated substrate through differing ring atoms without denoting a specific point of attachment, then all possible points are intended, whether through a carbon atom or, for example, a trivalent nitrogen atom. For example, the term “pyridyl” means 2-, 3-, or 4-pyridyl, the term “thienyl” means 2-, or 3-thienyl, and so forth.

In general the compounds of this invention can be made by processes which include processes analogous to those known in the chemical arts, particularly in light of the description contained herein. Certain processes for the manufacture of the compounds of this invention are provided as further features of the invention and are illustrated by the following reaction schemes. Other processes may be described in the experimental section.

The compounds of Formula I may be synthesized by methods similar to those disclosed in U.S. Pat. No. 5,338,740. Specific synthetic schemes for preparation of the compounds of Formula I are outlined below.

As an initial note, in the preparation of the Formula I compounds it is noted that some of the preparation methods useful for the preparation of the compounds described herein may require protection of remote functionality (e.g., primary amine, secondary amine, carboxyl in Formula I precursors). The need for such protection will vary depending on the nature of the remote functionality and the conditions of the preparation methods. The need for such protection is readily determined by one skilled in the art. The use of such protection/deprotection methods is also within the skill in the art. For a general description of protecting groups and their use, see T. W. Greene, Protective Groups in Organic Synthesis, John Wiley & Sons, New York, 1991.

For example, certain compounds contain primary amines or carboxylic acid functionalities which may interfere with reactions at other sites of the molecule if left unprotected. Accordingly, such functionalities may be protected by an appropriate protecting group which may be removed in a subsequent step. Suitable protecting groups for amine and carboxylic acid protection include those protecting groups commonly used in peptide synthesis (such as N-t-butoxycarbonyl, benzyloxycarbonyl, and 9-fluorenylmethylenoxycarbonyl for amines and lower alkyl or benzyl esters for carboxylic acids) which are generally not chemically reactive under the reaction conditions described and can typically be removed without chemically altering other functionality in the Formula I compound.

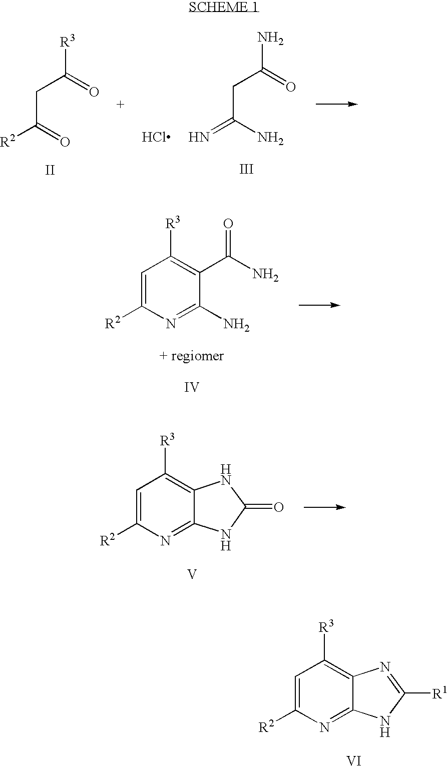

The Formula VI imidazo[4,5b]pyridine intermediate compounds wherein R¹, R² and R³ are as defined above may be prepared from the appropriate Formula IV compounds wherein R² and R³ are appropriate to achieve the desired Formula VI compound by a Hofmann rearrangement, cyclization and conversion of the urea V to the imidazole ring analogously to a procedure reported in Tetrahedron Lett. 1994, 35, 5775-5778.

For example, the Formula VI compounds may be prepared from the Formula V compounds by treatment with a di-substituted anhydride, selected to achieve the desired R¹ substituent, (e.g., propionic anhydride) at a temperature of about 15° C. to about 30° C., typically room temperature under an inert atmosphere, followed by addition of an alkyl acid e.g., propionic acid and an activating agent such as magnesium chloride. The resulting slurry is heated at temperatures of about 60° C. to about 180° C. for about six hours to about twenty-four hours followed by addition of a protic solvent such as methanol and continued heating at temperatures of about 30° C. to about 90° C. for about thirty minutes to about two hours resulting in the desired Formula VI 2, 5, 7 substituted imidazo[4,5b]pyridine intermediate compounds.

Alternatively, the Formula VI compounds may be prepared from the Formula V compounds by treatment with the appropriate alkyl acid such as propionic acid in a strong inorganic acid such as hydrochloric acid at elevated temperatures of about 150° C. to about 250° C. for about eight to about twenty-four hours in a sealed reactor to yield the desired Formula VI compound.

The Formula V compound may be prepared in two steps. A suitable inorganic base such potassium hydroxide in a protic solvent such as methanol is treated with the Formula III 2-amidino-acetamide (i.e., malonamamidine hydrochloride) at reduced temperatures of about 0° C. to about 20° C. followed by warming to about 15° C. to about 25° C. for at least about five minutes to about one hour. The desired Formula II compound, (wherein R² and R³ are selected to yield the desired Formula VI compound), is added to the mixture at a temperature of about ambient for about twenty-four to about forty-eight hours resulting in the desired Formula IV regioisomers.

Iodobenzene diacetate is added to the resulting Formula IV regioisomers mixture subsequent to the addition of more base e.g., potassium hydroxide and cooling to temperatures of about −20° C. to about 0° C. Following this the mixture is maintained at reduced temperatures for about one to about six hours followed by warming to ambient temperatures for a period of about eight to about twenty-four hours resulting in the desired Formula V imidazo-pyridin-one.

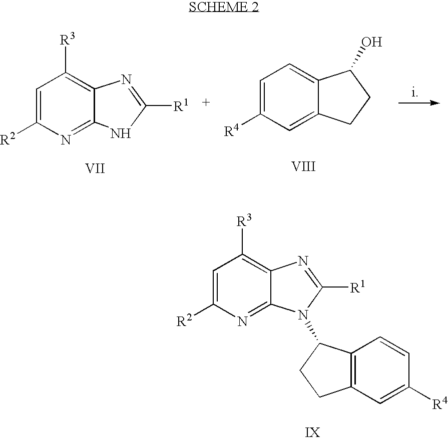

The Formula IX intermediate chiral compounds wherein R¹, R² and R³ are as defined above and R⁴ is bromine or 2-(1-trityl-1H-tetrazol-5-yl)phenyl)- may be prepared by condensation of VII and VIII under Mitsunobu conditions. The reaction of the appropriate compounds of Formula VII wherein R¹, R² and R³ are appropriate and the appropriately substituted (R)-indanol VIII proceeds with inversion of stereochemistry on the indane ring to achieve the desired Formula IX corresponding imidazo[4,5b]pyridine intermediate with the requisite (S)-configuration.

The Formula VII compounds are combined with triphenylphosphine and the appropriate Formula VIII indanol, wherein R⁴ is preferably bromo or 2-(1-trityl-1H-tetrazol-5-yl)phenyl)-, in an anhydrous, aprotic solvent such as THF or toluene under nitrogen. The mixture is cooled to a temperature below about 10° C. followed by addition of diethylazodicarboxylate (DEAD) and warming to a temperature of about 20° C. to about 30° C. for about eight to about twenty-four hours to provide the desired Formula IX conjugate intermediate.

Alternatively, the Formula VII compounds are combined with tributylphosphine, the appropriate Formula VIII indanol, and DEAD in an anhydrous solvent such as toluene or THF under nitrogen in the presence of an amine base such as diisopropylethyl amine at a temperature of about 50° C. to about 70° C. to yield the Formula IX intermediate.

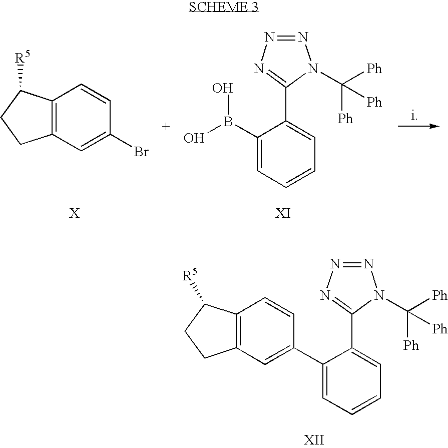

The Formula XII intermediate compounds wherein R⁵ is the 2,5,7-substituted 3H-imidazo[4,5-b]pyridine-1-yl moiety of the desired Formula I compound may be prepared from the corresponding Formula X bromo-compounds by a palladium catalyzed Suzuki reaction with the Formula XI compound as the coupling partner.

Triphenylphosphine in an aprotic solvent such as DME, toluene or DMF is deoxygenated with nitrogen for a sufficient time period e.g., 30 minutes. Palladium diacetate is added to the mixture and the mixture stirred for about 10 minutes to about two hours followed by addition of potassium carbonate, water and the Formula XI boronic acid. The mixture is exposed to elevated temperatures of about 50° C. to about reflux, preferably about 80° C. under an inert atmosphere for about six to about twenty-four hours to provide the desired Formula XII compound.

As appropriate, the triphenylmethyl (trityl) group may be removed from the tetrazole ring by deprotection with aqueous acid (e.g., sulfuric acid, hydrochloric acid) in a polar solvent such as acetonitrile or acetone. Alternatively, it can be removed by refluxing in a protic solvent such as methanol, or by using a reagent such as trimethylsilyl iodide in THF. The trityl protecting group on the tetrazole may either be attached to N-1 or N-2. In the final product, the hydrogen may be attached to either N-1 or N-2. For convenience, both the trityl group and H are shown as attached to N-1.

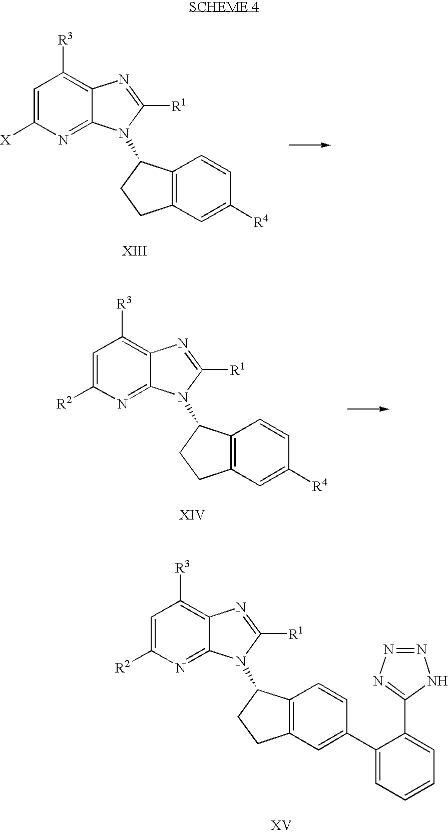

Scheme 4 illustrates that the C5 position of the imidazopyridine can be modified by procedures known to those skilled in the art. R⁴ in the Formula XIII compound is preferably bromo, but may also be a 2-(1-trityl-1H-tetrazol-5-yl)-phenyl substituent derived from prior Suzuki coupling. When both X and R⁴ are Br, coupling reactions generally favor reaction at the X substituent (e.g., Br) preferentially giving a single product with the R⁴ substituent (e.g., Br) intact. For example, for the Formula XIII compound wherein X is Br (or Cl), carbonylation in the presence of methanol yields compounds wherein X is COOMe, aryl cyanation yields compounds wherein X is CN and Sonogoshira coupling reactions with 2-substituted acetylenes yield compounds wherein X is an R² including an ethyne linkage. Other coupling procedures include using enolates as substrates to give compounds wherein X is an R² including a —CH₂CHOH linkage (e.g., —CH₂CHOH-alkyl). Thus, one may obtain a diversity of Formula XIII intermediates wherein X is Br, Cl, COOMe, CN, CHO, etc. that can be further modified into the compound XIV C5 R² substitutions as defined above by methods that are well known by those familiar with the art. For some analogs it is preferred to have the XIII intermediate R⁴ substituent as 2-(1-trityl-1H-tetrazol-5-yl)-phenyl prior to modification of the X substituent into the final R² moiety. Such derivatives may be prepared according to Scheme 2. Thus transformations from X to R² may take multiple steps to achieve the objective substitution.

Further, the Formula I compounds wherein R¹, R², and R³ are as defined above (depicted as Formula XV compounds) may be prepared from the Formula XIV compound, wherein R⁴ is either Br or 2-(1-trityl-1H-tetrazol-5-yl)-phenyl and R¹, R², and R³ are appropriate to achieve the desired Formula XV compound. When R⁴ is Br, various coupling reactions known to those skilled in the art such as Suzuki and Stille couplings may be used to form the biphenyl linkage and the final compound obtained by deprotection of the tetrazole Alternatively, if the Formula XIV compound R⁴ substituent is 2-(1-trityl-1H-tetrazol-5-yl)-phenyl, then removal of the trityl group is all that is required to obtain the Formula XV compound. Of course, although illustrated as one step those skilled in the art understand that the conversion can take several steps.

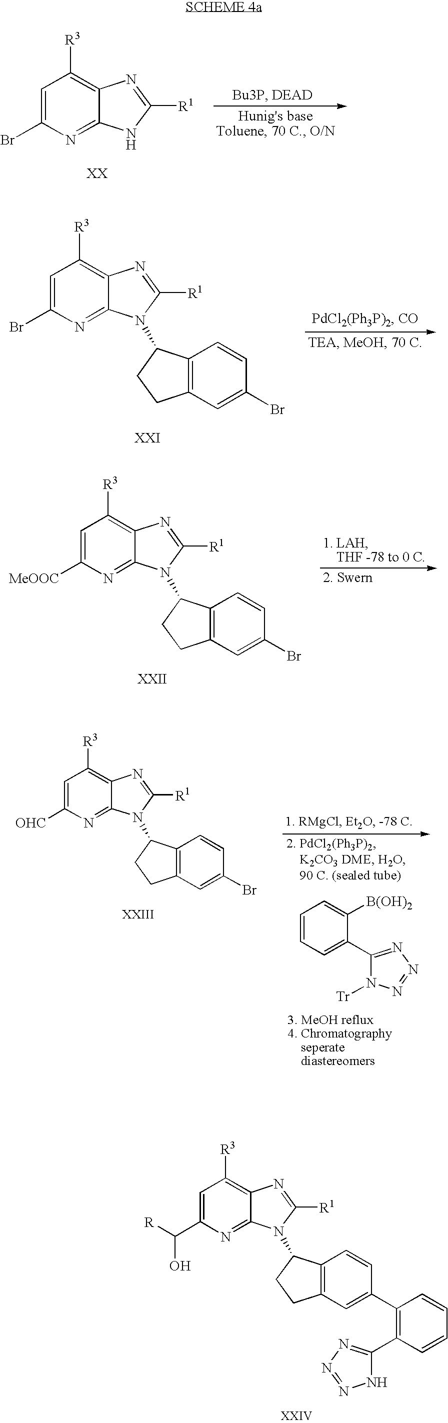

The Formula I compounds wherein R¹ and R³ are as defined above and R is an R² which includes a hydroxylmethylene linkage to the imidazopyridine core (depicted as Formula XXIV compounds) may be prepared from the Formula XXIII aldehyde, wherein R¹ and R³ are appropriate. The Formula XXIII aldehyde is reacted with a Grignard reagent followed by a coupling reaction, such as a Suzuki reaction, and cleavage of the trityl group to give the Formula XXIV compounds with the substituted hydroxymethylene moiety at C5.

The Formula XXIII aldehyde is dissolved in a polar, aprotic solvent such as ether or THF at reduced temperatures of about −78° C. to about 0° C. for about one hour to about six hours followed by reaction with the appropriate alkyl- or arylmagnesium halide.

The resulting compound may be conjugated at the aryl bromide with the tetrazolylphenyl moiety by a palladium catalyzed Suzuki reaction. Thus, it is treated with 2-(1-trityl-1H-tetrazol-5-yl)-phenyl boronic acid in the presence of a suitable catalyst such as palladium II acetate and triphenylphosphine in the presence of an inorganic base such as potassium carbonate.

Specifically, triphenylphosphine in an aprotic solvent such as DME is deoxygenated with nitrogen for a sufficient time period e.g., 30 minutes. A suitable catalyst such as palladium diacetate or palladium chloride is added to the mixture and the mixture stirred for about 10 minutes to about two hours followed by addition of potassium carbonate, water and 2-(1-trityl-1H-tetrazol-5-yl)-phenyl boronic acid. The mixture is exposed to elevated temperatures of about 50° C. to about reflux, preferably about 80° C. under an inert atmosphere for about six to about twenty-four hours. The trityl group is removed as described in Scheme III by methods previously outlined to provide the desired Formula XXIV compound.

The desired Formula XXIII aldehyde wherein R¹ and R³ are as defined above may be prepared from the desired Formula XXII compound, wherein R¹ and R³ are appropriate to achieve the desired Formula XXIII compound, by reduction followed by oxidation to the corresponding aldehyde.

The Formula XXII compound is treated as a cooled solution with a strong reducing agent such as lithium aluminum hydride in an anhydrous, aprotic solvent such as THF or ether at reduced temperatures of about −10° C. to about 15° C. for about 10 minutes to about one hour. The resulting alcohol is oxidized, for example by a Swern oxidation, with an oxidizing agent such as oxalyl chloride in the presence of an amine base such as triethylamine and DMSO. The reaction is prepared in an inert solvent such as dichloromethane at reduced temperatures of about −78° C. to about ambient for about thirty minutes to about two hours providing the Formula XXIII aldehyde.

The Formula XXII compound may be prepared from the appropriate Formula XXI compound by palladium-catalyzed carbonylation in the presence of methanol.

The Formula XXI compound is mixed with a palladium catalyst such as bis(triphenylphosphine)palladium(II) dichloride in a protic solvent such as methanol with an amine base such as triethylamine at an elevated temperature of about 50° C. to about reflux, preferably about 70° C. under carbon monoxide atmosphere for about ten to about two hundred fifty hours to provide the desired Formula XXII ester.

The Formula XXI compound may be prepared from the appropriate Formula XX compound by a Mitsunobu-like reaction.

The appropriate Formula XX compound, (R)-5-bromo-indan-1ol and tributylphosphine is cooled to a temperature of about −10° C. to about 15° C. and treated with diethylazadicarboxylate (DEAD). The mixture is allowed to warm to room temperature for about 30 minutes to about two hours followed by addition of a suitably selective amine base such as di-isopropylethylamine (Hunig's Base) at elevated temperatures of about 50° C. to about 100° C.

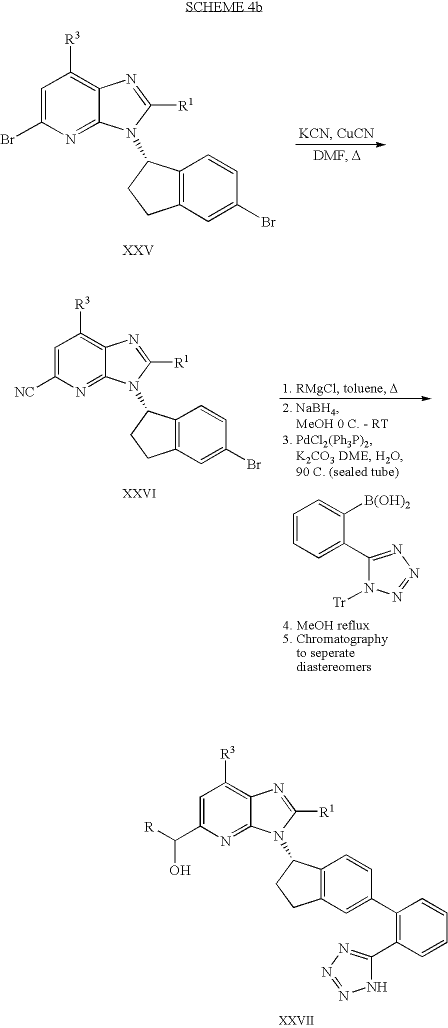

The Formula I compounds wherein R¹ and R³ are as defined above and R is an R² which includes a hydroxylmethylene linkage to the imidazopyridine core (depicted as Formula XXVII compounds) may be prepared from the Formula XXVI compound, wherein R¹ and R³ are appropriate to achieve the desired Formula XXVII compound by reaction with a Grignard reagent followed by reduction of the resulting ketone and a coupling reaction such as a Suzuki reaction, and cleavage of the trityl group.

The Formula XXVI cyano compound is dissolved in an anhydrous solvent such as toluene and reacted with an appropriate alkyl- or arylmagnesium halide at an elevated temperatures of about 40° C. to about 60° C.

The resulting compound is reduced with a hydride reducing agent such as sodium borohydride in a protic solvent such as methanol or lithium aluminum hydride in an aprotic solvent such as THF. The product is then coupled with the tetrazolphenyl moiety by a palladium catalyzed Suzuki reaction. Thus, it is treated with 2-(1-trityl-1H-tetrazol-5-yl)-phenyl boronic acid in the presence of a suitable catalyst such as palladium II acetate and triphenylphosphine in the presence of an inorganic base such as potassium carbonate.

Specifically, triphenylphosphine in an aprotic solvent such as DME is deoxygenated with nitrogen for a sufficient time period e.g., 30 minutes. A suitable catalyst such as palladium diacetate or palladium chloride is added to the mixture and the mixture stirred for about 10 minutes to about two hours followed by addition of potassium carbonate, water and 2-(1-trityl-1H-tetrazol-5-yl)-phenyl boronic acid. The mixture is exposed to elevated temperatures of about 50° C. to about reflux, preferably about 80° C. under an inert atmosphere for about six to about twenty-four hours.

As appropriate, the trityl group may be removed as described in Scheme III to provide the desired Formula XXVII compound.

The Formula XXVI compounds wherein R¹ and R³ are as defined above may be prepared from the Formula XXV compound, wherein R¹ and R³ are appropriate to achieve the desired Formula XXVI compound by a conventional aromatic nucleophilic substitution of a cyanide ion for the bromo substituent.

The Formula XXV bromo compound is mixed with potassium cyanide and copper(I) cyanide in an polar solvent such as DMF at elevated temperatures of about 100° C. to about 200° C., typically about 145° C. for about twelve hours to about twenty-four hours to provide the desired Formula XXVI cyano compound.

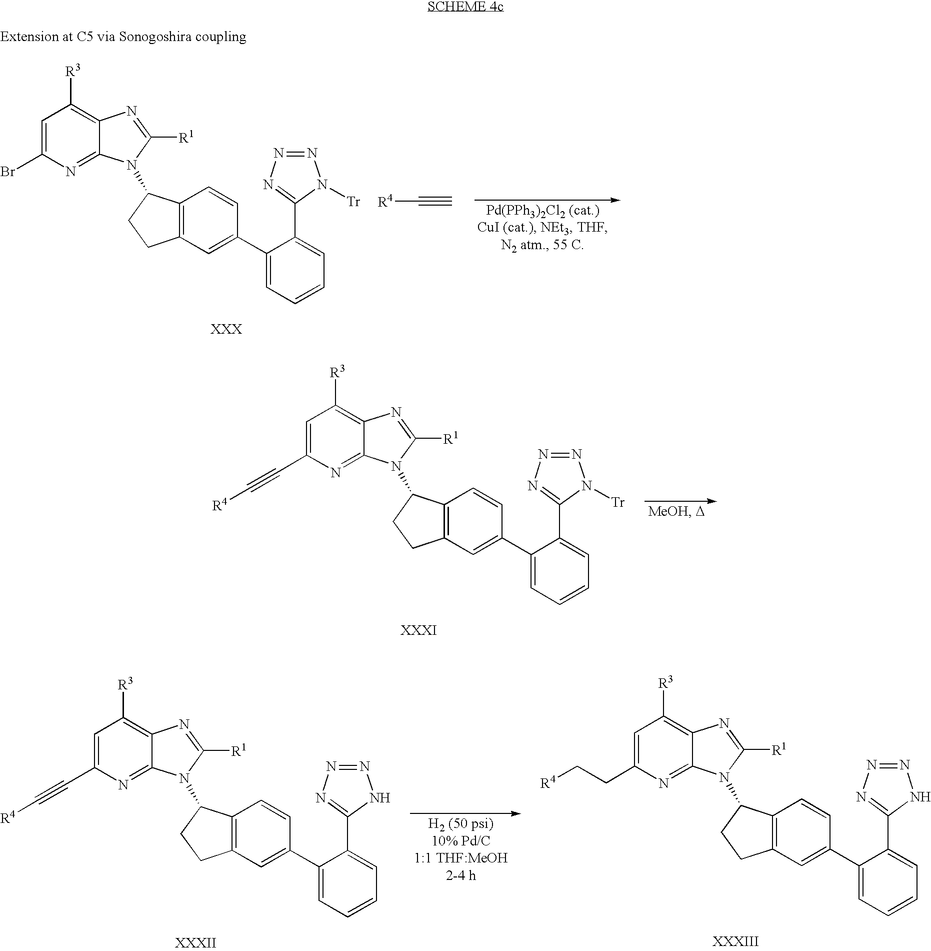

The Formula I compounds wherein R¹ and R³ are as defined above and R⁴ is an R² which includes an ethyl linkage to the imidazopyridine core (depicted as Formula XXXIII compounds) may be prepared via a Sonogoshira coupling followed by hydrogenation.

The Formula XXXIII compounds wherein R¹ and R³ are as defined above and R⁴ is an R² (which includes an ethyl linkage to the imidazopyridine core) may be prepared from the Formula XXXII compound, wherein R¹, R³ and R⁴ are appropriate to achieve the desired Formula XXXIII compound, by hydrogenation. The Formula XXXII compound is treated with a hydride source (e.g., 1 to 10 atmospheres of hydrogen gas, cyclohexene or ammonium formate) in the presence of a suitable catalyst (e.g., 5-20% palladium on carbon, palladium hydroxide; preferably 10% palladium on carbon) in a polar solvent (e.g., methanol, ethanol or ethyl acetate; preferably ethanol) at a temperature between about −78° C. and about 100° C., preferably ambient temperature, for 0.1 to 24 hours, preferably 1 hour.

The Formula XXXII compounds wherein R¹ and R³ are as defined above and R⁴ is an R² (which includes an ethyl linkage to the imidazopyridine core) may be prepared from the Formula XXXI compounds by deprotection using methods known to those skilled in the art. Briefly, the Formula XXXI compounds are heated at a temperature of about 30° C. and about reflux with a suitable alcohol such as methanol for about twelve hours to about 48 hours to remove the triphenylmethyl (trityl) moiety protecting group.

The Formula XXXI compounds wherein R¹ and R³ are as defined above and R⁴ is an R² (which includes an ethyl linkage to the imidazopyridine core) may be prepared from the Formula XXX bromo-imidazopyridine with the appropriate R⁴-acetylene compound by coupling reactions known to those skilled in the art. For example, the Formula XXX compound (in an anhydrous, polar solvent such as THF) is treated with a coupling catalyst such as bis(triphenylphosphine)palladium(II) dichloride and copper iodide in the presence of a base such as an amine base e.g., triethylamine. The desired R⁴-acetylene compound is added and the mixture is heated at elevated temperatures of about reflux under nitrogen for about two hours to about twelve hours.

Those skilled in the art will know that the reaction sequence can be varied, for example, the trityl moiety may be removed after hydrogenation of the alkyne.

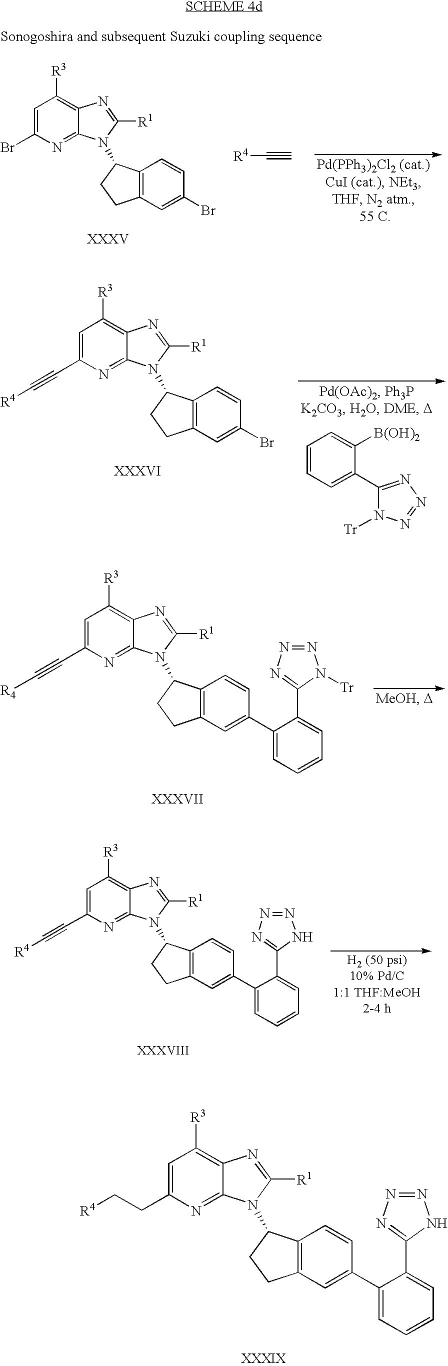

This scheme provides an alternative to Scheme 4c in that the tetrazolylphenyl substituent is added after the extension at the imidazopyridine C5 via Sonogoshira coupling. This alternative synthesis is a complementary method that may be preferred on large scale.

For the Formula XXXIX compounds the hydrogenation of the alkyne was generally performed after the Sonogashira coupling step and followed by the removal of the trityl group.

The Formula XXXIX compounds wherein R¹ and R³ are as defined above and R⁴ is an R² (which includes an ethyl linkage to the imidazopyridine core) may be prepared from the Formula XXXVIII compound, wherein R¹, R³ and R⁴ are appropriate to achieve the desired Formula XXXIX compound, by hydrogenation. The Formula XXXVIII compound is treated with a hydride source (e.g., 1 to 10 atmospheres of hydrogen gas, cyclohexene or ammonium formate) in the presence of a suitable catalyst (e.g., 5-20% palladium on carbon, palladium hydroxide; preferably 10% palladium on carbon) in a polar solvent (e.g., methanol, ethanol or ethyl acetate; preferably ethanol) at a temperature between about −78° C. and about 100° C., preferably ambient temperature, for 0.1 to 24 hours, preferably 1 hour.

The Formula XXXVIII compounds wherein R¹ and R³ are as defined above and R⁴ is an R² (which includes an alkyne linkage to the imidazopyridine core) may be prepared from the Formula XXXVII compounds by deprotection using methods known to those skilled in the art. Briefly, the Formula XXXVII compounds are heated at a temperature of about 30° C. and about reflux with a suitable alcohol such as methanol for about twelve hours to about 48 hours to remove the triphenylmethyl (trityl) moiety protecting group.

The Formula XXXVII compounds wherein R¹, R², and R⁴ are as defined above may be prepared from the Formula XXXVI compounds, wherein R¹, R², and R⁴ are selected to achieve the desired Formula XXXVII compounds, by treatment with triphenylphosphine and 2-(1-trityl-1H-tetrazol-5-yl)-phenyl boronic acid in the presence of a suitable catalyst such as palladium (II) acetate, and an inorganic base such as potassium carbonate.

Specifically, triphenylphosphine in an aprotic solvent such as DME is deoxygenated with nitrogen for a sufficient time period e.g., 30 minutes. A suitable catalyst such as palladium diacetate or palladium chloride is added to the mixture and the mixture stirred for about 10 minutes to about two hours followed by addition of potassium carbonate, water and 2-(1-trityl-1H-tetrazol-5-yl)-phenyl boronic acid. The mixture is exposed to elevated temperatures of about 50° C. to about reflux, preferably about 80° C. under an inert atmosphere for about six to about twenty-four hours.

The Formula XXXVI compounds wherein R¹ and R³ are as defined above and R⁴ is an R² (which includes an alkyne linkage to the imidazopyridine core) may be prepared from the Formula XXXV bromo-imidazopyridine with the appropriate R⁴-acetylene compound by coupling reactions known to those skilled in the art. For example, the Formula XXXV compound (in an anhydrous, polar solvent such as THF) is treated with a coupling catalyst such as bis(triphenylphosphine)palladium(II) dichloride and copper iodide in the presence of a base such as an amine base e.g., triethylamine. The desired R⁴-acetylene compound is added and the mixture is heated at elevated temperatures of about reflux under nitrogen for about two hours to about twelve hours.

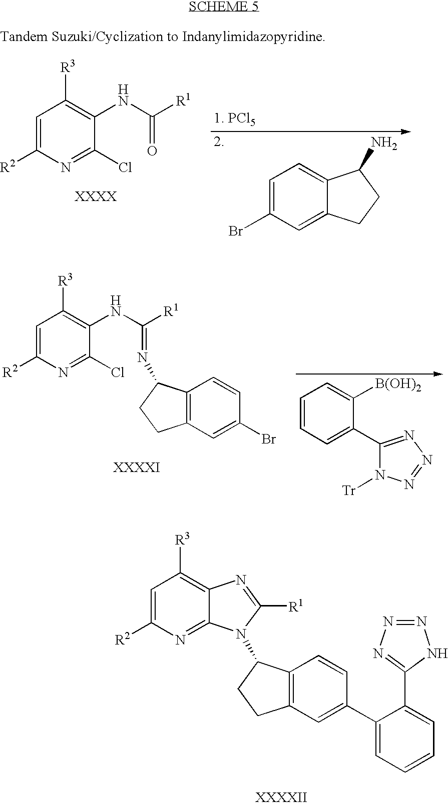

The Formula I compounds wherein R¹, R², and R³ are as defined above (depicted as Formula XXXXII compounds) may be prepared by a tandem Suzuki/cyclization procedure followed by removal of the trityl group.

Thus, the Formula XXXXII compounds wherein R¹, R², and R³ are as defined above may be prepared from the Formula XXXXI imidoylchloride compounds, wherein R¹, R², and R³ are selected to achieve the desired Formula XXXXII compounds, by treatment with triphenylphosphine and 2-(1-trityl-1H-tetrazol-5-yl)-phenyl boronic acid in the presence of a suitable catalyst such as palladium (II) acetate, and an inorganic base such as potassium carbonate.

The components are combined in a suitable aprotic solvent such as DME, under an inert gas such as nitrogen, with catalytic amount of water at room temperature followed by temperature elevation to about 40° C. to about reflux, preferably about 85° C. for about one to about six hours. S-Phos (2-dicyclohexylphosphino-2′,6′-dimethoxy-1,1′-biphenyl) and a suitable catalyst such as palladium (II) acetate are then added to the reaction mixture and the reaction is heated at an elevated temperature of about 40° C. to about reflux, preferably about 85° C. for about 10 to about 24 hours.

The trityl protection may be removed by methods such as those previously described.

The Formula XXXXI compounds wherein R¹, R², and R³ are as defined above may be prepared from the Formula XXXX 2,3,5,6-substituted pyridines by synthesis of an acyl halide followed by amination. The Formula XXXX compound is mixed with phosphorous pentachloride in an aprotic solvent such as DCM at an elevated temperature of about reflux for about one to about five hours to yield the intermediate imidoylchloride. The resulting imidoylchloride is combined at a low temperature of about 0° C. to about room temperature with the appropriate (S)-5-bromo-indan-1-ylamine and a suitable base such as an amine base e.g., triethylamine for about two to about 170 hours.

The starting materials and reagents for the above described Formula I compounds, are also readily available or can be easily synthesized by those skilled in the art using conventional methods of organic synthesis. For example, many of the compounds used herein, are related to, or are derived from compounds in which there is a large scientific interest and commercial need, and accordingly many such compounds are commercially available or are reported in the literature or are easily prepared from other commonly available substances by methods which are reported in the literature.

Cis/trans isomers may be separated by conventional techniques well known to those skilled in the art, for example, chromatography and fractional crystallisation.

Mixtures of stereoisomers may be separated by conventional techniques known to those skilled in the art. [see, for example, “Stereochemistry of Organic Compounds” by E L Eliel (Wiley, New York, 1994).]

Conventional techniques for the preparation/isolation of individual enantiomers include chiral synthesis from a suitable optically pure precursor.

Alternatively, the racemate (or a racemic precursor) may be reacted with a suitable optically active compound, for example, an alcohol, or, in the case where the compound of Formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Pharmaceutically acceptable salts of compounds of Formula I may be prepared by one or more of three methods:
(i) by reacting the compound of Formula I with the desired acid or base;
(ii) by removing an acid- or base-labile protecting group from a suitable precursor of the compound of Formula I or by ring-opening a suitable cyclic precursor, for example, a lactone or lactam, using the desired acid or base; or
(iii) by converting one salt of the compound of Formula I to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionization in the resulting salt may vary from completely ionized to almost non-ionized.

The compounds of this invention may also be used in conjunction with other pharmaceutical agents (e.g., antihypertensive and antidiabetic agents) for the treatment of the disease/conditions described herein.

The compounds of the present invention may be used in combination with antihypertensive agents and such antihypertensive activity is readily determined by those skilled in the art according to standard assays (e.g., blood pressure measurements). Exemplary antihypertensive agents include calcium channel blockers, angiotensin converting enzyme inhibitors (ACE inhibitors), antiotensin II receptor antagonists (ARB antagonists), Beta-adrenergic receptor blockers (beta- or β-blockers), Alpha-adrenergic receptor blockers (alpha- or α-blockers), vasodilators such as cerebral vasodilators, coronary vasodilators and peripheral vasodilators and diuretics.

The compounds of the present invention may be used in combination with antidiabetic agents and such anti-diabetic activity is readily determined by those skilled in the art according to standard assays known in the art. Examples of such antidiabetic agents include aldose reductase inhibitors, glucocorticoid receptor antagonists, glycogenolysis inhibitors, glycogen phosphorylase inhibitors, sorbitol dehydrogenase inhibitors, insulin, insulin analogs, insulinotropin, sulfonylureas and analogs, biguanides, imidazolines, insulin secretagogues, linogliride, glitazones, glucosidase inhibitors, acarbose, miglitol, emiglitate, voglibose, camiglibose, β-agonists, phosphodiesterase inhibitors, vanadate, vanadium complexes (e.g. Naglivan®), peroxovanadium complexes, amylin antagonists, glucagon antagonists, gluconeogenesis inhibitors, somatostatin analogs, antilipolytic agents, nicotinic acid, acipimox, pramlintide (Symlin™), and nateglinide

Preferred antidiabetic agents include chlorpropamide, glibenclamide, tolbutamide, tolazamide, acetohexamide, Glypizide®, glimepiride, repaglinide, meglitinide, metformin, phenformin, buformin, midaglizole, isaglidole, deriglidole, idazoxan, efaroxan, fluparoxan, ciglitazone, pioglitazone, englitazone, darglitazone, clomoxir or etomoxir.

The compounds of the present invention may be used in combination with cholesterol modulating agents (including cholesterol lowering agents) such as a lipase inhibitor, an HMG-CoA reductase inhibitor, an HMG-CoA synthase inhibitor, an HMG-CoA reductase gene expression inhibitor, an HMG-CoA synthase gene expression inhibitor, an MTP/Apo B secretion inhibitor, a CETP inhibitor, a bile acid absorption inhibitor, a cholesterol absorption inhibitor, a cholesterol synthesis inhibitor, a squalene synthetase inhibitor, a squalene epoxidase inhibitor, a squalene cyclase inhibitor, a combined squalene epoxidase/squalene cyclase inhibitor, a fibrate, niacin, an ion-exchange resin, an antioxidant, an ACAT inhibitor or a bile acid sequestrant.

The compounds of the present invention can be used in combination with anti-obesity agents. Such anti-obesity activity is readily determined by those skilled in the art according to standard assays known in the art. Suitable anti-obesity agents include phenylpropanolamine, ephedrine, pseudoephedrine, phentermine, β₃ adrenergic receptor agonists, apolipoprotein-B secretion/microsomal triglyceride transfer protein (apo-B/MTP) inhibitors, MCR-4 agonists, cholecystokinin-A (CCK-A) agonists, monoamine reuptake inhibitors (e.g., sibutramine), sympathomimetic agents, serotoninergic agents, cannabinoid receptor (CB-1) antagonists (e.g., rimonabant described in U.S. Pat. No. 5,624,941 (SR-141,716A), purine compounds, such as those described in US Patent Publication No. 2004/0092520; pyrazolo[1,5-a][1,3,5]triazine compounds, such as those described in U.S. Non-Provisional patent application Ser. No. 10/763,105 filed on Jan. 21, 2004; and bicyclic pyrazolyl and imidazolyl compounds, such as those described in U.S. Provisional Application No. 60/518,280 filed on Nov. 7, 2003), dopamine agonists (e.g., bromocriptine), melanocyte-stimulating hormone receptor analogs, 5HT2c agonists, melanin concentrating hormone antagonists, leptin (the OB protein), leptin analogs, leptin receptor agonists, galanin antagonists, lipase inhibitors (e.g., tetrahydrolipstatin, i.e. orlistat), bombesin agonists, anorectic agents (e.g., a bombesin agonist), Neuropeptide-γ antagonists, thyroxine, thyromimetic agents, dehydroepiandrosterones or analogs thereof, glucocorticoid receptor agonists or antagonists, orexin receptor antagonists, urocortin binding protein antagonists, glucagon-like peptide-1 receptor agonists, ciliary neurotrophic factors (e.g., Axokine™), human agouti-related proteins (AGRP), ghrelin receptor antagonists, histamine 3 receptor antagonists or inverse agonists, neuromedin U receptor agonists, and the like.

The compounds of this invention may also be used in combination with a lipase inhibitor. A lipase inhibitor is a compound that inhibits the metabolic cleavage of dietary triglycerides or plasma phospholipids into free fatty acids and the corresponding glycerides (e.g. EL, HL, etc.). Under normal physiological conditions, lipolysis occurs via a two-step process that involves acylation of an activated serine moiety of the lipase enzyme. This leads to the production of a fatty acid-lipase hemiacetal intermediate, which is then cleaved to release a diglyceride. Following further deacylation, the lipase-fatty acid intermediate is cleaved, resulting in free lipase, a glyceride and fatty acid. In the intestine, the resultant free fatty acids and monoglycerides are incorporated into bile acid-phospholipid micelles, which are subsequently absorbed at the level of the brush border of the small intestine. The micelles eventually enter the peripheral circulation as chylomicrons. Such lipase inhibition activity is readily determined by those skilled in the art according to standard assays (e.g., Methods Enzymol. 286: 190-231).

Pancreatic lipase mediates the metabolic cleavage of fatty acids from triglycerides at the 1- and 3-carbon positions. The primary site of the metabolism of ingested fats is in the duodenum and proximal jejunum by pancreatic lipase, which is usually secreted in vast excess of the amounts necessary for the breakdown of fats in the upper small intestine. Because pancreatic lipase is the primary enzyme required for the absorption of dietary triglycerides, inhibitors have utility in the treatment of obesity and the other related conditions. Such pancreatic lipase inhibition activity is readily determined by those skilled in the art according to standard assays (e.g., Methods Enzymol. 286: 190-231).

Gastric lipase is an immunologically distinct lipase that is responsible for approximately 10 to 40% of the digestion of dietary fats. Gastric lipase is secreted in response to mechanical stimulation, ingestion of food, the presence of a fatty meal or by sympathetic agents. Gastric lipolysis of ingested fats is of physiological importance in the provision of fatty acids needed to trigger pancreatic lipase activity in the intestine and is also of importance for fat absorption in a variety of physiological and pathological conditions associated with pancreatic insufficiency. See, for example, C. K. Abrams, et al., Gastroenterology, 92, 125 (1987). Such gastric lipase inhibition activity is readily determined by those skilled in the art according to standard assays (e.g., Methods Enzymol. 286: 190-231).

A variety of gastric and/or pancreatic lipase inhibitors are known to one of ordinary skill in the art.

In combination therapy treatment, both the compounds of this invention and the other drug therapies are administered to mammals (e.g., humans, male or female) by conventional methods.

The Formula I compounds of this invention, their prodrugs and the salts of such compounds and prodrugs are all adapted to therapeutic use as agents that mediate angiotensin II and PPARγ activity in mammals, particularly humans. Hence, these compounds are useful for the treatment of the various conditions in which the action of angiotensin II is implicated as described above. In addition, these compounds are useful for the treatment of the various conditions in which the action of PPARγ agonist activity is implicated such as are described above. Thus, by virtue of their combined angiotensin II activity and their PPARγ activity they are adapted to treat conditions such as for example, hypertension, type 2 diabetes, insulin resistance, hyperinsulinemia, hyperlipidemia, hypertriglyceridemia, metabolic syndrome, ir congestive heart failure.

It is believed that the renin-angiotensin system acts as a crucial regulatory mechanism in the control of homeostasis and fluid/electrolyte balance in mammals, including humans. Renin-angiotensin system activity has a direct influence on blood pressure and has been found to play an important role in congestive heart failure and in the development and maintenance of hypertension. Angiotensin II, an octapeptide hormone produced via the cleavage of angiotensin I by angiotensin converting enzyme, is a potent and direct arterial vasoconstrictor which increases vascular resistance and blood pressure and accordingly angiotensin II antagonists have a beneficial impact on such vascular resistant and blood pressure mediated diseases as hypertension and congestive heart failure and complications due to those conditions. Angiotensin II mediates its physiological actions through activation of two G-protein coupled receptors. The AT1 receptor is the principle receptor for the vasoconstrictor, proinflammatory, antinatriuretic and hypertrophic actions of Angiotensin II. The AT2 receptor is less well characterized, but is believed to act as a physiological regulator by opposing many of the AT1 mediated actions.

Given the ability of the Formula I compounds of this invention, their prodrugs and the salts of such compounds and prodrugs to impact PPARγ, they are of use in the treatment of type 2 diabetes and to decrease insulin resistance, hyperglycemia, hyperinsulinemia, and hypertriglyceridemia. The term PPARγ modulator refers to compounds which modulate peroxisome proliferator activator receptor gamma (PPARγ) activity in mammals, particularly humans.

It is believed the compounds of this invention, by activating the PPARγ receptor, stimulate transcription of key genes involved in lipid and glucose metabolism such as those in fatty acid oxidation and also those involved in glucose transport and metabolism. Enhanced glucose uptake into peripheral tissues in response to insulin signaling as well as suppression of inflammatory cytokines released from visceral adipose tissues constitutes some of the pleiotropic effects of this mechanistic class. By virtue of their activity, these agents reduce the proinflammatory burden on the vasculature and reduce atherosclerotic development and macrovascular events in diabetic patients.

Given the positive correlation between triglycerides, LDL cholesterol, and their associated apolipoproteins in blood with the development of cardiovascular, cerebral vascular and peripheral vascular diseases, the Formula I compounds of this invention, their prodrugs and the salts of such compounds and prodrugs, by virtue of their pharmacologic action, are useful for the prevention, arrestment and/or regression of atherosclerosis and its associated disease states. These include cardiovascular disorders (e.g., angina, cardiac ischemia and myocardial infarction) and complications due to cardiovascular disease.

Thus, given the ability of the Formula I compounds of this invention, their prodrugs and the salts of such compounds and prodrugs to reduce plasma glucose, insulin and triglycerides, they are of use in the treatment of diabetes.

The utility of the Formula I compounds of the invention, their prodrugs and the salts of such compounds and prodrugs as medical agents in the treatment of the above described disease/conditions in mammals (e.g. humans, male or female) is demonstrated by the activity of the compounds of this invention in conventional in vitro and in vivo assays described below. The in vivo assays (with appropriate modifications within the skill in the art) may be used to determine the activity of other agents as well as the compounds of this invention. Such assays also provide a means whereby the activities of the Formula I compounds of this invention, their prodrugs and the salts of such compounds and prodrugs (or the other agents described herein) can be compared to each other and with the activities of other known compounds. The results of these comparisons are useful for determining dosage levels in mammals, including humans, for the treatment of such diseases.

The following protocols may of course be varied by those skilled in the art.

### PPARγ Receptor Agonist Activity In Vitro Assay

PPARγ activity was determined in two in vitro assay formats. Human 6× His tagged PPARγ-LBD (aa 206-477, Genbank accession number NM_{—}138712.1) was prepared for use in a scintillation proximity assay (SPA) to determine binding affinity. This pure protein was incubated with yttrium silicate SPA beads, various compound dilutions and 3H-darglitazone as the competing radioligand and incubated for 3 hours to allow equilibration. Total binding was determined in the absence of compound and non-specific binding in the presence of 100 μM rosiglitazone. Plates were read on a TopCount (Perkin Elmer) and concentration-response curves constructed using commercial curve fitting software. The Ki was determined via interpolation and use of the Cheng-Prussof equation.

PPARγ agonist activity may be determined by a cell line transiently transfected with PPARγ bound to a DNA binding domain that controls luciferase expression. The degree of receptor agonism is measured by the amount of luciferase activity after compound treatment. The ratio of treated cells over vehicle control cells provides a measure of fold activation and allows an EC₅₀ (shown in tables I and II as SPA EC₅₀) and % activation to be calculated

A human hepatocellular carcinoma cell line (HepG2) was subcultured into dishes and transiently transfected with DNA to allow expression of human PPARγ LBD bound to Gal4 DBD. Luciferase reporter gene and β-galactosidase control genes were co-transfected to enable activity to be measured. After 24 hours, cells were exposed to compounds in the concentration range of 0.1 nM-100 μM and left for a further 24 hours. Luciferase activity is determined by luminometry and PPARγ activation is expressed as a ratio of luciferase activity to β-galactosidase activity to account for transfection efficiency. Potency is described by an EC₅₀ defined as the concentration of compound producing receptor activation equivalent to 50% of the maximum for that compound (shown in Table I as transfection EC50). The maximum fold activation is a measure of efficacy and is expressed as a percentage of a reference full agonist run in the same assay. Efficacy is described by % activation of receptor relative to a standard thiazolidinedione (TZD) full agonist.

### Angiotensin II Type 1 Receptor Antagonist Activity In Vitro Assay

Angiotensin II receptor antagonist activity may be determined using commercially available flashplates coated with the AT1 or AT2 receptor which provide a rapid and high throughput means to evaluate compounds compared to traditional filtration based assays (Regina M. Van Der Hee et al., Journal of Biomolecular Screening 10(2); 2005; 118-126).

AT1 receptor affinity was determined in a radioligand binding format using commercially available flashplate technology. Human recombinant AT1 receptor were coated on the wells of a 96 well flashplate and test compounds were diluted and added to wells at a final concentration range of 50 μM to 1 μM. Total binding was determined in the absence of compound and non-specific binding in the presence of 10 μM cold Angiotensin II. ¹²⁵I labeled Sar¹, Ile⁸-Angiotensin II was added to all wells at Kd concentration and left for 2 hours at room temperature to equilibrate. Plates were read on a TopCount (Perkin Elmer) and concentration-response curves constructed using commercial curve fitting software. The Ki was determined via interpolation and the use of the Cheng-Prussof equation.

TABLE 1
In Vitro assays for AT1 antagonist activity (IC50) and
PPARγ activity (EC50 and % activation
compared to full agonist response).

| | | PPARγ | |
|---|---|---|---|
| | AT1 IC50 | Transfection | PPARγ % |
| Example | (nM) | EC50 (nM) | activation |
| 1 | 4.5 | 90 | 27 |
| 2 | 3.5 | 105 | 29 |
| 3 | 1.6 | 665 | 32 |
| 4 | 2.2 | 295 | 32 |
| 5 | 2.9 | 290 | 48 |
| 6 | 10 | 275 | 46 |
| 8a | 1.3 | 60 | 16 |
| 8b | 1.5 | 100 | 25 |
| 9 | 2.7 | 60 | 27 |
| 10 | 1.6 | 145 | 27 |
| 11 | 16 | 440 | 39 |
| 12 | 0.7 | 460 | 17 |
| 13 | 2.5 | 435 | 26 |
| 13a | 4.1 | 415 | 24 |
| 14 | 5 | 410 | 21 |
| 15 | 1.7 | 85 | 19 |
| 16 | 2.9 | 250 | 20 |
| 17 | 8.8 | 65 | 24 |
| 18 | 5.4 | 85 | 29 |
| 19 | 7.2 | 105 | 20 |
| 20 | 5.7 | 70 | 31 |
| 21 | 3.1 | 100 | 23 |
| 22 | 2.2 | 460 | 28 |
| 23 | 4.8 | 220 | 26 |
| 24 | 2.1 | 425 | 28 |
| 25 | 2.4 | 180 | 26 |
| 26 | 10 | 380 | 26 |
| 27 | 2.0 | 485 | 21 |
| 28 | 2.2 | 330 | 15 |
| 29 | 2.9 | 195 | 31 |
| 30 | 6.0 | 160 | 28 |
| 31 | 6.9 | 45 | 63 |
| 32 | 2.1 | 510 | 20 |
| 33 | 2.1 | 400 | 23 |
| 34 | 2.1 | 595 | 12 |
| 35 | 0.5 | 390 | 23 |
| 36 | 1.2 | 215 | 30 |
| 37 | 0.5 | 270 | 24 |
| 38 | 2.0 | 215 | 21 |
| 39 | 1.1 | 135 | 24 |
| 40 | 0.8 | 770 | 20 |
| 41 | 0.9 | 965 | 21 |
| 42 | 1.1 | 155 | 29 |
| 43 | 1.0 | 840 | 20 |
| 44 | 1.1 | 475 | 38 |
| 45 | 1.1 | 140 | 32 |
| 46 | 1.2 | 170 | 26 |
| 47 | 1.3 | 550 | 34 |
| 48 | 1.1 | 190 | 21 |
| 49 | 1.6 | 815 | 18 |
| 50 | 1.7 | 415 | 30 |
| 51 | 2.1 | 130 | 36 |
| 52 | 1.3 | 290 | 36 |
| 53 | 1.4 | 215 | 30 |
| 54 | 1.0 | 535 | 26 |
| 55 | 2.4 | 150 | 38 |
| 56 | 2.5 | 110 | 31 |
| 57 | 2.5 | 285 | 28 |
| 58 | 3.8 | 200 | 31 |
| 59 | 2.9 | 215 | 30 |
| 60 | 22 | 525 | 21 |
| 61 | 5 | 410 | 21 |
| 62 | 1.4 | 70 | 50 |
| 63 | 2.4 | 295 | 27 |
| 64 | 1.9 | 95 | 21 |
| 65 | 2.3 | 245 | 22 |
| 66 | 6 | 435 | 22 |
| 67 | 5.1 | 110 | 23 |
| 68 | 6.5 | 45 | 34 |
| 69 | 4.0 | 30 | 36 |

### In Vivo Assay

The dual pharmacology exhibited by this compound necessitates the use of two in vivo models to adequately define efficacy. The SHR (Spontaneously Hypertensive Rat) is a Wistar derived strain that demonstrates arterial hypertension of polygenic origin and has been shown over several decades to predict human antihypertensive efficacy. The rats are surgically implanted with radio-telemetry transmitters to allow capture of conscious, unrestrained systolic and diastolic BP as well as heart rate.

The male Zucker Diabetic Fatty rat (ZDF♂) is a substrain derived from the Zucker fa/fa rat that possesses a spontaneous mutation in the leptin receptor (fa gene). Homozygous expression of this mutation leads to a phenotype of hyperphagia, obesity, insulin resistance, hyperglycemia, and hypertriglyceridemia resulting in uncontrolled diabetes, β-cell failure, severe nephropathy, and proteinuria by 16 weeks of age.

### Blood Pressure Reduction In Vivo Assay

In vivo studies were conducted to show the effectiveness of a compound in reducing blood pressure due to its angiotensin II receptor antagonist activity, and its ability to induce insulin sensitization in a diabetic rat due to its PPARγ agonist activity.

Spontaneously hypertensive rats (SHR) were implanted with radiotelemetry devices capable of measuring blood pressure (BP) via an aortic cannula and transmitting the data to a receiving pad under the cage. Animals were conscious and had access to food and water ad libitum while blood pressure was monitored continuously. Rats were dosed with vehicle and monitored for 24 hours to establish baseline before administering compound and measuring changes in blood pressure for another 24 hours. Changes in blood pressure were calculated over time and compared to vehicle controls.

TABLE 2

| | Example | Example | | |
|---|---|---|---|---|
| SHR rat | 10 | 13 | Example 16 | Example 13a |
| Maximal fall in | −27 | −46 | −37 | −28 |
| Mean Blood | | | | |
| Pressure after | | | | |
| single dose of 10 | | | | |
| mg/kg p.o. | | | | |
| (mmHg) | | | | |
| Duration of activity | >20 h | >20 h | >20 h | >20 h |

### Glucose Lowering In Vivo Assay

The hypoglycemic activity of the compounds of this invention can be determined by the amount of test compound that delays or suppresses elevations in glucose levels relative to a vehicle without test compound in male ZDF rats.

Male Zucker Diabetic Fatty (ZDF/Crl-Lepr^{fa}) rats were obtained from Charles River Laboratories (Wilmington, Mass.). Rats were pair housed under a 12-h light/dark cycle with free access to water and Purina 5008 rat chow (Protein 26.8%, fat 16.7%, carbohydrates 56.5% kcal/vol; Purina Mills, Richmond, Ind.). Prior to the onset of diabetic hyperglycemia (fed blood glucose<200 mg/dl) rats were randomly allocated to groups based on HOMA values. The Homeostasis Model Assessment (HOMA) calculation estimates peripheral insulin resistance from corresponding insulin and glucose measurements. Rats were administered a once daily oral dose for 28 days with suspensions of vehicle alone (1.5% carboxymethyl-cellulose plus 0.2% Tween 20), vehicle plus test compound in the range 0.1-100 mg/kg. At the end of the study, an oral glucose tolerance test (OGTT, 2 g/kg dextrose) was conducted to measure glucose excursion. Tail venipuncture in conscious, 1 hr fasted animals at baseline and weekly thereafter, provided sample for blood glucose, serum insulin, triglycerides, cholesterol, and FFA measurements. Glucose levels were determined with a HemoCue Glucose Monitor (Ryan Diagnostics), insulin was determined by ELISA (Alpco), and lipids were determined by Cobas Mira Analyzer (Roche) and by enzymatic assays (Wako). Lipoprotein cholesterol was determined by FPLC following the method of Kieft et al. (Kieft K A, Bocan T M, Krause B R. Rapid on-line determination of cholesterol distribution among plasma lipoproteins after high-performance gel filtration chromatography. J Lipid Res 1991; 32(5): 859-66). Briefly, plasma was separated on a Superose 6HR 10/30 column (Pharmacia) and cholesterol was then measured in-line utilizing a post-column enzymatic cholesterol assay (Roche). Percent of total area for each lipoprotein fraction were calculated. These percent areas were then multiplied by the total plasma cholesterol concentration, yielding the cholesterol concentration in each fraction. Efficacy was determined by comparison to untreated vehicle controls and reference agents and active compounds selected for further evaluation.

TABLE 3

| | Example | Example | Example | |
|---|---|---|---|---|
| ZDF male rat | 10 | 13 | 16 | Example 13a |
| Blood Glucose (1 h | 239 | 120 | 114 | 112 |
| Fast) after single | | | | |
| daily dose of 10 mg/kg | | | | |
| p.o. for 28 | | | | |
| days (mg/dl) | | | | |
| Difference from | −133 | −469 | −475 | −477 |
| Vehicle Control | | | | |
| (mg/dl) | | | | |

### Insulin, Triglyceride, and Cholesterol Levels In Vivo Assay

The compounds of the present invention are readily adapted to clinical use as hyperinsulinemia reversing agents, triglyceride lowering agents and hypocholesterolemic agents. Such activity can be determined by the amount of test compound that reduces insulin, triglycerides or cholesterol levels relative to a control vehicle without test compound in male ob/ob mice.

Since the concentration of cholesterol in blood is closely related to the development of cardiovascular, cerebral vascular or peripheral vascular disorders, the compounds of this invention, by virtue of their hypocholesterolemic action, prevent, arrest and/or regress atherosclerosis.

Since the concentration of insulin in blood is related to the promotion of vascular cell growth and increased renal sodium retention, (in addition to the other actions, e.g., promotion of glucose utilization) and these functions are known causes of hypertension, the compounds of this invention, by virtue of their hypoinsulinemic action, prevent, arrest and/or regress hypertension.

Since the concentration of triglycerides in blood contributes to the overall levels of blood lipids, the compounds of this invention, by virtue of their triglyceride lowering and/or free fatty acid lowering activity prevent, arrest and/or regress hyperlipidemia.

Free fatty acids contribute to the overall level of blood lipids and independently have been negatively correlated with insulin sensitivity in a variety of physiologic and pathologic states.

Five to eight week old male C57BL/6J-ob/ob mice (obtained from Jackson Laboratory, Bar Harbor, Me.) are housed five per cage under standard animal care practices and fed standard rodent diet ad libitum. After a one week acclimation period, the animals are weighed and 25 microliters of blood are collected from the retro-orbital sinus prior to any treatment. The blood sample is immediately diluted 1:5 with saline containing 0.025% sodium heparin, and held on ice for plasma glucose analysis.

Animals are assigned to treatment groups so that each group has a similar mean for plasma glucose concentration. The compound to be tested is administered by oral gavage as an about 0.02% to 2.0% solution (weight/volume (w/v)) in either (1) 10% DMSO/0.1% Pluronic® P105 Block Copolymer Surfactant (BASF Corporation, Parsippany, N.J.) in 0.1% saline without pH adjustment or (2) 0.25% w/v methylcellulose in water without pH adjustment. Alternatively, the compound to be tested can be administered by oral gavage dissolved in or in suspension in neat PEG 400. Single daily dosing (s.i.d.) or twice daily dosing (b.i.d.) is maintained for 1 to, for example, 15 days. Control mice receive the 10% DMSO/0.1% Pluronic® P105 in 0.1% saline without pH adjustment or the 0.25% w/v methylcellulose in water without pH adjustment, or the neat PEG 400 without pH adjustment.

Three hours after the last dose is administered, the animals are sacrificed by decapitation and trunk blood is collected into 0.5 ml serum separator tubes containing 3.6 mg of a 1:1 weight/weight sodium fluoride:potassium oxalate mixture. The freshly collected samples are centrifuged for two minutes at 10,000×g at room temperature, and the serum supernatant is transferred and diluted 1:1 volume/volume with a 1TIU/ml aprotinin solution in 0.1% saline without pH adjustment.

The diluted serum samples are then stored at −80° C. until analysis. The thawed, diluted serum samples are analyzed for insulin, triglycerides, free fatty acids and cholesterol levels. Serum insulin concentration is determined using Equate® RIA INSULIN kits (double antibody method; as specified by the manufacturer) available from Binax, South Portland, Me. The inter assay coefficient of variation is ≦10%. Serum triglycerides are determined using the Abbott VP™ and VP Super System® Autoanalyzer (Abbott Laboratories, Irving, Tex.), or the Abbott Spectrum CCX™ (Abbott Laboratories, Irving, Tex.) using the A-Gent™ Triglycerides Test reagent system (Abbott Laboratories, Diagnostics Division, Irving, Tex.) (lipase-coupled enzyme method; a modification of the method of Sampson, et al., Clinical Chemistry 21: 1983 (1975)). Serum total cholesterol levels are determined using the Abbott VP™ and VP Super System® Autoanalyzer (Abbott Laboratories, Irving, Tex.), and A-Gent™ Cholesterol Test reagent system (cholesterol esterase-coupled enzyme method; a modification of the method of Allain, et al. Clinical Chemistry 20: 470 (1974)) using 100 and 300 mg/dl standards. Serum free fatty acid concentration is determined utilizing a kit from WAKO (Osaka, Japan), as adapted for use with the Abbott VP™ and VP Super System® Autoanalyzer (Abbott Laboratories, Irving, Tex.), or the Abbott Spectrum CCX™ (Abbott Laboratories, Irving, Tex.). Serum insulin, triglycerides, free fatty acids and total cholesterol levels are then calculated by the equations: serum insulin (μU/ml)=sample value×2; serum triglycerides (mg/dl)=sample value×2; serum total cholesterol (mg/dl)=sample value×2; serum free fatty acid (μEq/l)=sample value×2; where 2 is the dilution factor.

The animals dosed with vehicle maintain substantially unchanged, elevated serum insulin (e.g., 275 μU/ml), serum triglycerides (e.g., 235 mg/dl), serum free fatty acid (1500 mEq/ml) and serum total cholesterol (e.g., 190 mg/dl) levels. The serum insulin, triglycerides, free fatty acid and total cholesterol lowering activity of the test compounds are determined by statistical analysis (unpaired t-test) of the mean serum insulin, triglycerides, and total cholesterol concentration between the test compound group and the vehicle-treated control group.

### Energy Expenditure

### Obesity In Vivo Assay

As would be appreciated by those skilled in the relevant art, during increased energy expenditure, animals generally consume more oxygen. In addition, metabolic fuels such as, for example, glucose and fatty acids, are oxidized to CO₂ and H₂O with the concomitant evolution of heat, commonly referred to in the art as thermogenesis. Thus, the measurement of oxygen consumption in animals, including humans and companion animals, is an indirect measure of thermogenesis. Indirect calorimetry is commonly used in animals, e.g., humans, by those skilled in the relevant art to measure such energy expenditures.

Those skilled in the art understand that increased energy expenditure and the concomitant burning of metabolic fuels resulting in the production of heat may be efficacious with respect to the treatment of, e.g., obesity.

The ability of the Formula I compounds to generate a thermogenic response may be demonstrated according to the following protocol: This in vivo screen is designed to evaluate the efficacy of compounds that are PPAR agonists, using as an efficacy endpoint measurement of whole body oxygen consumption. The protocol involves: (a) dosing fatty Zucker rats for about 6 days, and (b) measuring oxygen consumption. Male fatty Zucker rats having a body weight range of from about 400 g to about 500 g are housed for from about 3 to about 7 days in individual cages under standard laboratory conditions prior to the initiation of the study. A compound of this invention and a vehicle is administered by oral gavage as a single daily dose given between about 3 p.m. to about 6 p.m. for about 6 days. A compound of this invention is dissolved in vehicle containing about 0.25% of methyl cellulose. The dosing volume is about 1 ml.

About 1 day after the last dose of the compound is administered, oxygen consumption is measured using an open circuit, indirect calorimeter (Oxymax, Columbus Instruments, Columbus, Ohio 43204). The Oxymax gas sensors are calibrated with N₂ gas and a gas mixture (about 0.5% of CO₂, about 20.5% of O₂, about 79% of N₂) before each experiment. The subject rats are removed from their home cages and their body weights recorded. The rats are placed into the sealed chambers (43×43×10 cm) of the Oxymax, the chambers are placed in the activity monitors, and the air flow rate through the chambers is then set at from about 1.6 L/min to about 1.7 L/min. The Oxymax software then calculates the oxygen consumption (mL/kg/h) by the rats based on the flow rate of air through the chambers and the difference in oxygen content at the inlet and output ports. The activity monitors have 15 infrared light beams spaced about one inch apart on each axis, and ambulatory activity is recorded when two consecutive beams are broken, and the results are recorded as counts.

Oxygen consumption and ambulatory activity are measured about every 10 min for from about 5 h to about 6.5 h. Resting oxygen consumption is calculated on individual rats by averaging the values excluding the first 5 values and the values obtained during time periods where ambulatory activity exceeds about 100 counts.

Administration of the compounds of this invention can be via any method which delivers a compound of this invention systemically and/or locally. These methods include oral routes, parenteral, intraduodenal routes, etc. Generally, the compounds of this invention are administered orally, but parenteral administration (e.g., intravenous, intramuscular, subcutaneous or intramedullary) may be utilized, for example, where oral administration is inappropriate for the target or where the patient is unable to ingest the drug.

For administration to human patients, oral daily dose of the compounds herein may be in the range 1 mg to 500 mg depending, of course, on the mode of administration. An oral daily dose is in the range of 3 mg to 250 mg may be used. A further oral daily dose is in the range of 5 mg to 180 mg. The total daily dose may be administered in single or divided doses and may, at the physician's discretion, fall outside of the typical ranges given herein.

A dosage of the combination pharmaceutical agents to be used in conjunction with the Formula I compounds is used that is effective for the indication being treated. Such dosages can be determined by standard assays such as those referenced above and provided herein. The combination agents may be administered simultaneously or sequentially in any order.

These dosages are based on an average human subject having a weight of about 60 kg to 70 kg. The physician will readily be able to determine doses for subjects whose weight falls outside this range, such as infants and the elderly.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regiments for administration of the chemotherapeutic agent are well-known in the relevant art and would be understood to be encompassed by the skilled artisan once provided the teachings disclosed herein.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The compounds described herein may be administered as a formulation comprising a pharmaceutically effective amount of a compound of Formula I, in association with one or more pharmaceutically acceptable excipients. The term “carrier” or “excipient” herein means any substance, not itself a therapeutic agent, used as a diluent, adjuvant, or vehicle for delivery of a therapeutic agent to a subject or added to a pharmaceutical composition to improve its handling or storage properties or to permit or facilitate formation of a solid dosage form such a tablet, capsule, or a solution or suspension suitable for oral parenteral, intradermal, subcutaneous, or topical application. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition. Acceptable excipients include stearic acid, magnesium stearate, magnesium oxide, sodium and calcium salts of phosphoric and sulfuric acids, magnesium carbonate, talc, gelatin, acacia gum, sodium alginate, pectin, dextrin, mannitol, sorbitol, lactose, sucrose, starches, gelatin, cellulosic materials, such as cellulose esters of alkanoic acids and cellulose alkyl esters, low melting wax, cocoa butter or powder, polymers such as polyvinyl-pyrrolidone, polyvinyl alcohol, and polyethylene glycols, and other pharmaceutical acceptable materials. Examples of excipients and their use may be found in Remington's Pharmaceutical Sciences, 20th Edition (Lippincott Williams & Wilkins, 2000). The choice of excipient will to a large extent depend on factors such as the particular mode of administration, the effect of the excipient on solubility and stability, and the nature of the dosage form.

The compounds herein may be formulated for oral, buccal, intranasal, parenteral (e.g., intravenous, intramuscular or subcutaneous) or rectal administration or in a form suitable for administration by inhalation. The compounds of the invention may also be formulated for sustained delivery.

Methods of preparing various pharmaceutical compositions with a certain amount of active ingredient are known, or will be apparent in light of this disclosure, to those skilled in this art. For examples of methods of preparing pharmaceutical compositions see Remington's Pharmaceutical Sciences, 20th Edition (Lippincott Williams & Wilkins, 2000).

Pharmaceutical compositions according to the invention may contain 0.1%-95% of the compound(s) of this invention, preferably 1%-70%. In any event, the composition or Formulation to be administered will contain a quantity of a compound(s) according to the invention in an amount effective to treat the disease/condition of the subject being treated, e.g., atherosclerosis.

Since the present invention has an aspect that relates to the treatment of the disease/conditions described herein with a combination of active ingredients which may be administered separately, the invention also relates to combining separate pharmaceutical compositions in kit form. The kit comprises two separate pharmaceutical compositions: a compound of Formula I a prodrug thereof or a salt of such compound or prodrug and a second compound as described above. The kit comprises means for containing the separate compositions such as a container, a divided bottle or a divided foil packet. Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of Formula I compound can consist of one tablet or capsule while a daily dose of the second compound can consist of several tablets or capsules and vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

The compounds of this invention either alone or in combination with each other or other compounds generally will be administered in a convenient formulation. The following formulation examples only are illustrative and are not intended to limit the scope of the present invention.

In the formulations which follow, “active ingredient” means a compound of this invention.

Formulation 1: Gelatin Capsules
Hard gelatin capsules are prepared using the following:

| | Quantity |
|---|---|
| Ingredient | (mg/capsule) |
| Active ingredient | 0.25-100 |
| Starch, NF | 0-650 |
| Starch flowable powder | 0-50 |
| Silicone fluid 350 centistokes | 0-15 |

A tablet Formulation is prepared using the ingredients below:

Formulation 2: Tablets

| Ingredient | Quantity (mg/tablet) |
|---|---|
| Active ingredient | 0.25-100 |
| Cellulose, microcrystalline | 200-650 |
| Silicon dioxide, fumed | 10-650 |
| Stearate acid | 5-15 |

The components are blended and compressed to form tablets.

Alternatively, tablets each containing 0.25-100 mg of active ingredients are made up as follows:

Formulation 3: Tablets

| Ingredient | Quantity (mg/tablet) |
|---|---|
| Active ingredient | 0.25-100 |
| Starch | 45 |
| Cellulose, microcrystalline | 35 |
| Polyvinylpyrrolidone (as 10% solution in | 4 |
| water) | |
| Sodium carboxymethyl cellulose | 4.5 |
| Magnesium stearate | 0.5 |
| Talc | 1 |

The active ingredients, starch, and cellulose are passed through a No. 45 mesh U.S. sieve and mixed thoroughly. The solution of polyvinylpyrrolidone is mixed with the resultant powders which are then passed through a No. 14 mesh U.S. sieve. The granules so produced are dried at 50°-60° C. and passed through a No. 18 mesh U.S. sieve. The sodium carboxymethyl starch, magnesium stearate, and talc, previously passed through a No. 60 U.S. sieve, are then added to the granules which, after mixing, are compressed on a tablet machine to yield tablets.

Suspensions each containing 0.25-100 mg of active ingredient per 5 ml dose are made as follows:

Formulation 4: Suspensions

| Ingredient | Quantity (mg/5 ml) | |
|---|---|---|
| Active ingredient | 0.25-100 | mg |
| Sodium carboxymethyl cellulose | 50 | mg |
| Syrup | 1.25 | mg |
| Benzoic acid solution | 0.10 | mL |
| Flavor | q.v. | |
| Color | q.v. | |
| Purified Water to | 5 | mL |

The active ingredient is passed through a No. 45 mesh U.S. sieve and mixed with the sodium carboxymethyl cellulose and syrup to form smooth paste. The benzoic acid solution, flavor, and color are diluted with some of the water and added, with stirring. Sufficient water is then added to produce the required volume.

An aerosol solution is prepared containing the following ingredients:

Formulation 5: Aerosol

| | Quantity (% by |
|---|---|
| Ingredient | weight) |
| Active ingredient | 0.25 |
| Ethanol | 25.75 |
| Propellant 22 (Chlorodifluoromethane) | 70.00 |

The active ingredient is mixed with ethanol and the mixture added to a portion of the propellant 22, cooled to 30° C., and transferred to a filling device. The required amount is then fed to a stainless steel container and diluted with the remaining propellant. The valve units are then fitted to the container.

Suppositories are prepared as follows:

Formulation 6: Suppositories

| | Quantity |
|---|---|
| Ingredient | (mg/suppository) |
| Active ingredient | 250 |
| Saturated fatty acid glycerides | 2,000 |

The active ingredient is passed through a No. 60 mesh U.S. sieve and suspended in the saturated fatty acid glycerides previously melted using the minimal necessary heat. The mixture is then poured into a suppository mold of nominal 2 g capacity and allowed to cool.

An intravenous Formulation is prepared as follows:

Formulation 7: Intravenous Solution

| Ingredient | Quantity | |
|---|---|---|
| Active ingredient dissolved in ethanol 1% | 20 | mg |
| Intralipid ™ emulsion | 1,000 | mL |

The solution of the above ingredients is intravenously administered to a patient at a rate of about 1 mL per minute.

Soft gelatin capsules are prepared using the following:

Formulation 8: Soft Gelatin Capsule with Oil Formulation

| Ingredient | Quantity (mg/capsule) |
|---|---|
| Active ingredient | 10-500 |
| Olive Oil or Miglyol ™ Oil | 500-1000 |

The active ingredient above may also be a combination of agents.

### General Experimental Procedures

All chemicals, reagents and solvents were purchased from commercial sources where available and used without further purification. Proton nuclear magnetic spectroscopy (1H-NMR) was recorded with a 400 MHz Varian spectrometer. Chemical shifts are expressed in parts per million downfield from tetramethylsilane. The peak shapes are denoted as follows: s, singlet; d, doublet; t, triplet, q, quartet; m, multiplet; bs=broad singlet. Mass spectrometry (MS) was performed via atmospheric pressure chemical ionization (APCI) or electron scatter (ES) ionization sources. Silica Gel chromatography was performed primarily using a medium pressure Biotage system using columns pre-packaged by various commercial vendors including Biotage. Alternatively, column chromatography was performed with either Baker Silica Gel (40 μm) (J. T. Baker, Phillipsburg, N.J.) or Silica Gel 60 (EM Sciences, Gibbstown, N.J.) in glass columns under low nitrogen pressure. Microanalyses were performed by Quantitative Technologies Inc. and were within 0.4% of the calculated values. The terms “concentrated” and “evaporated” refer to removal of solvent at water aspirator pressure on a rotary evaporator with a bath temperature of less than 45° C. Reactions conducted at “0-20° C.” or “0-25° C.” were conducted with initial cooling of the vessel in an insulated ice bath which was allowed to warm to room temperature over several hours. The abbreviation “min” and “h” stand for “minutes” and “hours” respectively.

### X-Ray Powder Diffraction

The X-ray powder diffraction patterns of all compounds were carried out on a Bruker D5000 diffractometer using CuK_{α} radiation. The instrument was equipped with a fine focus X-ray tube. The tube voltage and amperage were set to 40 kV and 40 mA, respectively. The divergence and scattering slits were set at 1 mm and the receiving slit was set at 0.6 mm. Diffracted radiation was detected by a Kevex PSI detector. A theta-two theta continuous scan at 2.4°/min (1 sec/0.04° step) from 3.0 to 40° 2θ was used. An alumina standard was analyzed to check the instrument alignment. Data were collected and analyzed using Bruker axs software Version 7.0. Samples were prepared for analysis by placing them in a quartz holder. It should be noted that Bruker Instruments purchased Siemans; thus, a Bruker D5000 instrument is essentially the same as a Siemans D5000.

To perform an X-ray diffraction measurement on a Bruker D8 Discover X-ray powder diffractometer with GADDS CS used for measurements reported herein, the sample is typically placed into a cavity in the middle of the silicon sample holder. The sample powder is pressed by a glass slide or equivalent to ensure a random surface and proper sample height. The sample holder is then placed into the diffractometer and the powder X-ray diffraction pattern is collected using the instrumental parameters specified above. Measurement differences associated with such X-ray powder diffraction analyses result from a variety of factors including: (a) errors in sample preparation (e.g., sample height), (b) instrument errors, (c) calibration errors, (d) operator errors (including those errors present when determining the peak locations), and (e) the nature of the material (e.g. preferred orientation errors). Calibration errors and sample height errors often result in a shift of all the peaks in the same direction. Small differences in sample height when using a flat holder will lead to large displacements in XRPD peak positions. A systematic study showed that a sample height difference of 1 mm could lead to peak shifts as high as 1° 2θ (Chen et al.; J Pharmaceutical and Biomedical Analysis, 2001; 26, 63). These shifts can be identified from the X-ray diffractogram and can be eliminated by compensating for the shift (applying a systematic correction factor to all peak position values) or recalibrating the instrument. As mentioned above, it is possible to rectify differences in measurements from the various instruments by applying a systematic correction factor to bring the peak positions into agreement. In general, this correction factor will bring the measured peak positions into agreement with the expected peak positions and may be in the range of the expected 2θ value±0.2° 2θ.

### Preparation of 5-bromo-2-ethyl-7-methyl-imidazo[4,5-b]pyridine

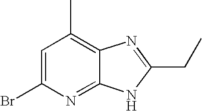

### Step 1. Preparation of 2,3-diamino-4-methylpyridine

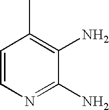

3-amino-4-methyl-2-nitro pyridine (20 g, 130 mmol) was dissolved in 400 mL methanol. Raney nickel (RaNi, 5 g, 58 mmol) was added and stirred under hydrogen gas (50 psi) for 14 hours. Solvent was removed in vacuo and the residue (15 g) was used without further purification (93% yield). ¹H NMR (400 MHz, DMSO-D6) δ ppm 2.0 (s, 3H) 4.3 (s, 2H) 5.2 (s, 2H) 6.2 (d, J=5.5 Hz, 1H) 7.1 (d, J=5.1 Hz, 1H).

### Step 2. Preparation of 2-ethyl-7-methylimidazo-(4,5-b)pyridine

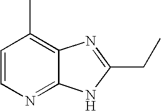

4-Methyl-pyridine-2,3-diamine (24 g, 195 mmol) was dissolved in propionic acid (25 mL, 270 mmol) and heated at 130° C. for 12 hours. As starting material and product were observed, heating continued an additional 24 h. 500 g of polyphosphoric acid and 50 mL propionic acid were added and the reaction heated at 80° C. for 24 hours. Reaction progress was monitored by mass spectroscopy (M+1=162.0). After starting material mass was consumed, the reaction mixture was poured over ice, and pH basified with solid NaOH. Aqueous solution extracted with EtOAc (5×75 mL). Solvent was removed in vacuo and the residue purified by silica gel chromatography to give 2-ethyl-7-methylimidazo-(4,5-b)pyridine (7.7 g, 24% yield). ¹H NMR (400 MHz, DMSO-D6) δ ppm 1.3 (q, J=7.7 Hz, 3H) 2.5 (s, 3H) 2.8 (m, 2H) 6.9 (d, J=4.9 Hz, 1H) 8.0 (m, 1H) 12.5 (d, J=99.8 Hz, 1H)

### Step 3. Preparation of 2-ethyl-7-methylimidazo-(4,5-b)pyridine-4-oxide

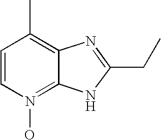

MCPBA (16 g, 72 mmol) was added to a solution of 2-ethyl-7-methylimidazo-(4,5-b)pyridine (7.7 g 48 mmol) in CHCl₃ (100 mL) and refluxed for 6 hours. Solvent was removed in vacuo and purified by silica gel chromatography using 0-30% MeOH/CHCl₃ to obtain the desired N-oxide. The product contained residual m-chlorobenzoic acid. A second column gave 2-ethyl-7-methylimidazo-(4,5-b)pyridine-4-oxide 6.2 grams of >90% pure material 1H NMR (400 MHz, DMSO-D6) δ ppm 1.3 (t, J=7.5 Hz, 3H) 2.4 (s, 3H) 2.8 (q, J=7.6 Hz, 2H) 3.3 (s, 1H) 6.9 (d, J=4.1 Hz, 1H) 7.9 (d, J=6.2 Hz, 1H), (M+1=178.0).

### Step 4. Preparation of 5-chloro-2-ethyl-7-methyl-imidazo[4,5-b]pyridine

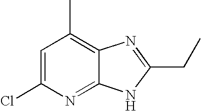

A mixture of 2-ethyl-7-methylimidazo(4,5-b)-pyridine-4-oxide (6.2 g, 35 mmol) in CHCl₃ (5 mL) was treated with POCl₃ (30 mL) and heated to 80° C. for 1 hour. The reaction mixture was poured over ice and adjusted to pH 10 with NH₄O, and then extracted with EtOAc (3×100 mL). Organic layers were combined and washed with brine. Solvent was removed in vacuo and crude material (6.6 g) was used without further purification (M+1=196.0/198.0). ¹H NMR (400 MHz, DMSO-D6) δ ppm 1.3 (q, J=7.5 Hz, 3H) 2.4 (s, 3H) 2.8 (m, 2H) 7.0 (s, 1H) 12.7 (d, J=72.2 Hz, 1H)

### Step 5. Preparation of 5-bromo-2-ethyl-7-methyl-imidazo[4,5-b]pyridine

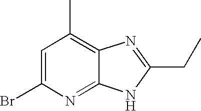

5-Chloro-2-ethyl-7-methyl-imidazo[4,5-b]pyridine (6.5 g, 33 mmol) was treated with 30% HBr/HOAc and heated at 10° C. for 16 hours. The suspension was poured over ice, neutralized with NH₄OH and extracted with EtOAc (5×30 mL). Continued addition of HBr/AcOH resulted in very slow conversion (by LCMS monitoring) but reaction seemed to be “clean”. Literature prep suggests 100° C. for 19 hours, but this procedure was difficult to follow due to the volatile nature of HBr. Significant pressure can develop if the flask remains closed. After repeated additions of fresh HBr/HOAc the reaction was quenched with ice, pH rendered basic, and extracted with EtOAc (5×75 mL). Organic layers combined and evaporated with SiO2 and loaded onto a column. Eluted with 50-100% EtOAc/Hex to give 5-bromo-2-ethyl-7-methyl-imidazo[4,5-b]pyridine (5 g, 60%). (M+1=242.1/242.1). 3.8:1 ratio bromide to chloride. 1H NMR (400 MHz, CHLOROFORM-D) δ ppm 1.5 (t, J=7.7 Hz, 3H) 2.6 (s, 3H) 3.1 (q, J=7.6 Hz, 2H) 7.2 (s, 1H)

### Preparation 1(S)-5-bromo-indan-1-yl-amine

### Step 1. (R)-5-bromo-indan-1-ol

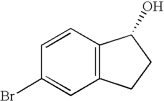

To a 22 L 5-necked RBF was charged 5-bromo-1-indanone (1.3 Kg, 6.159 mol), anhydrous THF (10 L) and (S)-methyl-CBS-oxazaborolidine (1 M in toluene, 950 ml, 0.95 mol). The mixture was cooled to −10° C. under N₂ and was added borane-methylsulfide (10.0 M, 850 ml, 8.5 mol) over 1 h while maintaining the temperature ˜below −5° C. The mixture was stirred at −10° C. to 0° C. for 3 h, cooled to −5° C. and quenched with water (5 L) at such a rate to maintain reaction temperature ˜5° C. The mixture was then extracted with EtOAc (4 L) and the aqueous layer was re-extracted with EtOAc (3×3 L). The combined organic extracts were washed with brine (4 L), dried over MgSO₄, filtered and concentrated to give brown solid. The crude product was passed through a short silica gel column (4 L silica gel packed with 1% Et₃N in hexanes, eluted with EtOAc/hexane (1/4)), the filtrate was concentrated and the residue was slurred with 10% EtOAc in hexanes, filtered then dried to give 871.0 g off-white solid as (R)-5-bromo-indan-1-ol. The mother liquors were re-concentrated, slurred with 10% EtOAc in hexanes and filtered to give another 210.0 g yellow solid as (R)-5-bromo-indan-1-ol (yield: 1081.0 g; 82%): ¹H NMR (CDCl3) consistent with product. Anal. calcd for C₉H₉BrO: C, 50.73; H, 4.28. Found: C, 50.31; H, 4.34.

### Step 2: (S)-1-azido-5-bromo-2,3-dihydro-1H-indene

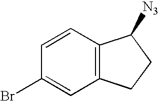

A solution of (R)-5-bromo-indan-1-ol (345.0 g, 1.619 mol) in toluene (2.5 L) was cooled in an ice bath under N₂ and treated with diphenylphosphoric azide (DPPA, 455.0 ml, 2.108 mol) in one portion followed by a solution of 1,8-diazabicyclo[5,4,0]undec-7-ene (DBU, 340 ml, 2.273 mol) in toluene (660 ml). The reaction temperature was kept between 3 to 10° C. during 3 h of addition and the mixture was warmed to 15° C. over next 3 h (TLC indicated no starting material). The mixture was diluted with EtOAc (2 L), washed with water (2×2 L), brine (2 L) and the organic layer was dried over MgSO₄, filtered then concentrated to give 669 g dark oil. The crude product was purified by silica gel column (packed with 1% Et₃N in hexanes, eluted with hexanes). (S)-1-azido-5-bromo-2,3-dihydro-1H-indene was obtained as an oil (375.4 g, 97%): ¹H NMR (CDCl3) was consistent with product.

### Step 3. (S)-5-bromo-2,3-dihydro-1H-inden-1-amine

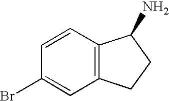

A solution of (S)-1-Azido-5-bromo-indan (375.4 g, 1.577 mol) in methanol (6.0 L) was treated with SnCl₂.2H₂O (640.4 g, 2.838 mol). The mixture was stirred at room temperature over night (TLC indicated no starting material), concentrated to dryness. The residue was treated with 2N NaOH (8 L), extracted with EtOAc (4×4 L). The combined organic extracts were filtered through celite, washed with 1 N HCl (3 L×4), followed by water (2 L). The aqueous layers were pooled, basified to pH 11 with cold saturated NaOH solution, extracted with EtOAc (3×4 L), the combined organic extracts were dried over MgSO₄, filtered then concentrated. (S)-5-bromo-2,3-dihydro-1H-inden-1-amine (302.0 g, 90%) was obtained as a yellow oil which solidified in fridge: ¹H NMR (CDCl3) consistent with product; MS: 213.84 (M+H)⁺. Anal. calcd for C₉H₁₀BrN: C, 50.97; H, 4.75; N, 6.60. Found: C, 51.26; H, 4.74; N, 6.71.

### Preparation 2 5-Bromo-2-ethyl-7-methyl-3-{(S)-5-[2-(1-trityl-1H-tetrazol-5-yl)-phenyl]-indan-1-yl}-3H-imidazo[4,5-b]pyridine

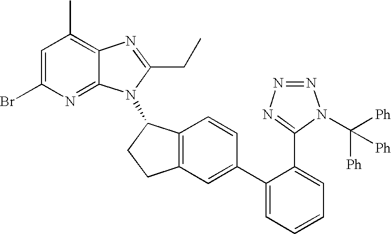

5-Bromo-2-ethyl-7-methyl-3H-imidazo[4,5-b]pyridine (8.6 g, 35.7 mmol), triphenyl phosphine (10 g, 39 mmol) and (S)-5-[2-(1-trityl-1H-tetrazol-5-yl)-phenyl]-indan-1-ol (19.5 g, 37.5 mmol) were dissolved in toluene (70 mL). DIAD (5.5 mL, 28.5 mmol) in heptane (70 mL) was added dropwise. The reaction was stirred at 23° C. for 16 hours. The solid was filtered and washed with 50% toluene/heptane. A white solid (17.9 g) was obtained which was discarded. The filtrate was treated with Celite® and then washed with 1M citric acid. The suspension was filtered to remove the viscous Celite®, and the layers separated. The organic layer was washed with 1M citric acid, brine, dried over MgSO₄ and the solvent was concentrated in vacuo to give 5-bromo-2-ethyl-7-methyl-3-{(S)-5-[2-(1-trityl-1H-tetrazol-5-yl)-phenyl]-indan-1-yl}-3H-imidazo[4,5-b]pyridine (18.8 g, 70.8% yield). HPLC 9.20 min. 65.0% pure; This batch was combined with 2 other batches (30.4 g) and was purified via silica gel chromatography (320 g), 9 cm×18 cm glass column and a gradient (0.1% Et₃N, 30% EtOAc/Heptane—0.1% Et₃N, 60% EtOAc/Heptane) as eluent to give 5-Bromo-2-ethyl-7-methyl-3-{5-[2-(1-trityl-1H-tetrazol-5-yl)-phenyl]-indan-1-yl}-3H-imidazo[4,5-b]pyridine (12.3 g). HPLC 9.20 min. 91.0%; NMR (DMSO) δ 0.81 (t, 3H), 2.49 (s, 3H), 2.62 (m, 3H), 2.78 (m, 2H), 3.12 (m, 2H), 6.61 (d, 1H), 6.83 (m, 7H), 7.07 (s, 1H), 7.35 (m, 9H), 7.50 (m, 4H), 7.75 (d, 1H). MS 744 [M+H], 500 [M−H].

### EXPERIMENTAL

HPLC method A refers to the following conditions:

Column: Symmetry C18, 4.6×150 mm

Mobile phase: A: water+0.1% TFA; B: CH₃CN+0.1% TFA

Flow: 1 mL/min

Gradient: 90% A to 10% A in 15 min, hold for 5 min, go back to 90% A in 1 min and maintain at 90% A for 4 min

Detection: UV at 220 nm

Injection: 10 uL

### Example 1

### 3-((1S)-5-(2-(1H-tetrazol-5-yl)phenyl)-2,3-dihydro-1H-inden-1-yl)-5-benzyl-2-ethyl-7-methyl-3H-imidazo[4,5-b]pyridine

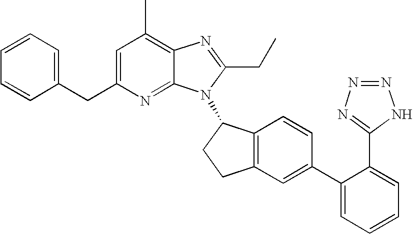

### Step 1. 2-Amino-6-benzyl-4-methylnicotinamide

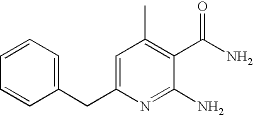

A homogenous solution of potassium hydroxide (1.44 g, 25.6 mmol) in MeOH (25 mL) was treated with malonamamidine hydrochloride (3.20 g, 23.3 mmol) that was added in one portion. The slurry was stirred for 10 minutes and then 1-phenylpentane-2,4-dione (4.20 g, 23.3 mmol) was added. Additional MeOH was added (50 mL) over two hours to maintain a stirrable slurry. Stirring continued for 18 h at RT. Water (15 mL) was added and mixture cooled in an ice bath for 1 h. Solid was separated by filtration. An approximate 60:40 mixture of 2-amino-6-benzyl-4-methylnicotinamide and 2-amino-4-benzyl-6-methylnicotinamide was obtained (2.44 g, 43%): ¹H NMR (400 MHz, DMSO-d₆) δ ppm 2.14, 2.16 (s, 3 h), 3.79, 3.86 (s, 2H), 5.60, 5.67 (s, 1H), 6.16, 6.32 (s, 1H), 7.13-7.31 (m, 5H), 7.50, 7.56 (bs, 1H), 7.67, 7.85 (bs, 1H); CIMS: 242.1 (APCI)+, 240.0 (APCI)−.

### Step 2. 5-benzyl-7-methyl-1H-imidazo[4,5-b]pyridin-2(3H)-one

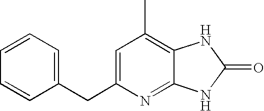

An approximate 60:40 mixture of 2-amino-6-benzyl-4-methylnicotinamide and 2-amino-4-benzyl-6-methylnicotinamide (1.80 g, 7.46 mmol) was added to a cold (0° C.) solution of potassium hydroxide (0.837 g, 14.9 mmol) in MeOH (25 mL). (Diacetoxyiodo)benzene (2.40 g, 7.46 mmol) was added with additional MeOH (15 mL). The mixture was stirred under a nitrogen atmosphere in an ice-bath and slowly warmed to RT over 4 h. The mixture was cooled in an ice bath for 30 min. A solid precipitate was separated by vacuum filtration and washed with ether and then air dried. An approximate 3:2 mixture of 5-benzyl-7-methyl-1H-imidazo[4,5-b]pyridin-2(3H)-one and 7-benzyl-5-methyl-1H-imidazo[4,5-b]pyridin-2(3H)-one was obtained (1.62 g, 91% yield): ¹H NMR (400 MHz, DMSO-d₆) δ ppm 2.22, 2.29 (s, 3H), 3.92, 3.93 (s, 2H), 6.60, 6.68 (s, 1H), 7.12-7.32 (m, 5H), 11.11 (bs, 2H); CIMS: 240.0 (APCI)+, 238.0 (APCI)−.

### Step 3. 5-benzyl-2-ethyl-7-methyl-3H-imidazo[4,5-b]pyridine

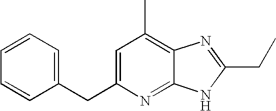

An approximate 3:2 mixture of 5-benzyl-7-methyl-1H-imidazo[4,5-b]pyridin-2(3H)-one and 7-benzyl-5-methyl-1H-imidazo[4,5-b]pyridin-2(3H)-one (1.50 g, 6.27 mmol) was slurried in propionic anhydride (4.85 mL) at RT and maintained under a nitrogen atmosphere. Propionic acid (2.81 mL) was added followed by magnesium chloride (0.597 g, 6.27 mmol). The thick slurry was heated at 120° C. for 18 h. Methanol (5 mL) was added to the reaction and the mixture maintained at 60° C. for 1 h. The mixture was cooled to RT that resulted in a thick slurry. The mixture was concentrated to yield a solid residue that was purified by MPLC eluting with ethyl acetate in hexanes. An approximate 3:2 mixture of 5-benzyl-2-ethyl-7-methyl-3H-imidazo[4,5-b]pyridine and 7-benzyl-2-ethyl-5-methyl-3H-imidazo[4,5-b]pyridine was obtained (1.07 g, 68% yield): ¹H NMR (400 MHz, DMSO-d₆) δ ppm 1.26-1.36 (m, 3H), 2.45, 2.43 (s, 3H), 2.74-2.89 (m, 2H), 3.92, 4.05, 4.15, 4.20 (s, 2H), 6.78, 6.87, 6.90 (s, 1H), 7.13-7.21 (m, 1H), 7.22-7.35 (m, 4H); CIMS: 252.1 (APCI)+, 250.1 (APCI)−; HPLC: 35.35%; Rt=8.185 min; 58.52%; Rt=8.431 min, method A.

### Step 4. (S)-5-benzyl-3-(5-bromo-2,3-dihydro-1H-inden-1-yl)-2-ethyl-7-methyl-3H-imidazo[4,5-b]pyridine

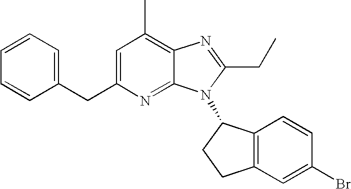

(R)-5-bromo-2,3-dihydro-1H-inden-1-ol (0.900 g, 4.22 mmol), an approximate 3:2 mixture of 5-benzyl-2-ethyl-7-methyl-3H-imidazo[4,5-b]pyridine and 7-benzyl-2-ethyl-5-methyl-3H-imidazo[4,5-b]pyridine (1.06 g, 4.22 mmol), and PPh₃ (1.66 g, 6.33 mmol) were stirred in THF (40 mL) under a nitrogen atmosphere. The mixture was cooled to 0° C. and a solution of diethylazodicarboxylate (DEAD, 0.997 mL, 6.33 mmol) in THF (2 mL) was added dropwise. The mixture was stirred overnight while allowing mixture to warm to RT. The mixture was concentrated to a dark oil and purified by MPLC eluting with ethyl acetate in hexanes. From this purification, (S)-5-benzyl-3-(5-bromo-2,3-dihydro-1H-inden-1-yl)-2-ethyl-7-methyl-3H-imidazo[4,5-b]pyridine was obtained pure as a white solid (1.06 g, 56% yield): ¹H NMR (400 MHz, DMSO-d₆) δ ppm 1.31 (t, J=7.56 Hz, 3H), 2.44 (s, 3H), 2.54-2.73 (m, 2H), 2.72-3.00 (m, 2H), 3.00-3.13 (m, 1H), 3.88 (s, 2H), 6.19 (t, J=8.17 Hz, 1H), 6.76 (d, J=8.05 Hz, 1H), 6.89 (s, 1H), 7.04 (d, J=7.08 Hz, 2H), 7.08-7.21 (m, 3H), 7.26 (dd, J=7.93, 1.83 Hz, 1H), 7.60 (d, J=1.71 Hz, 1H); CIMS: 448.1 (APCI)+.

### Step 5. 5-benzyl-2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridine

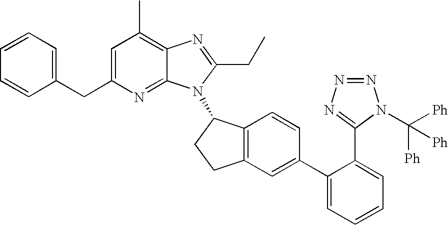

Triphenylphosphine (0.176 g, 0.672 mmol) was dissolved in DME (15 mL) and the mixture was deoxygenated by bubbling nitrogen through the solution for 30 min. Pd(OAc)₂ (30.2 mg, 0.134 mL) was added and the mixture stirred for 30 min. 2-(1-trityl-1H-tetrazol-5-yl)phenylboronic acid (1.16 g, 2.69 mmol), (S)-5-benzyl-3-(5-bromo-2,3-dihydro-1H-inden-1-yl)-2-ethyl-7-methyl-3H-imidazo[4,5-b]pyridine (0.600 g, 1.34 mmol), potassium carbonate (0.464 g, 3.36 mmol), and water (0.060 mL, 3.36 mmol) were added. The mixture was heated at 80° C. under a nitrogen atmosphere for 18 h. The mixture was allowed to cool and diluted with EtOAc (15 mL), and then filtered through a pad of celite. Filter cake was washed with EtOAc (2×10 mL). The mixture was concentrated and the residue was purified by MPLC eluting with ethyl acetate in hexanes. From this purification, 5-benzyl-2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridine (0.479 g, 47% yield) was obtained as a foam: CIMS: 754.3 (APCI)+, 510.2 (APCI)−.

### Step 6. 3-((1S)-5-(2-(1H-tetrazol-5-yl)phenyl)-2,3-dihydro-1H-inden-1-yl)-5-benzyl-2-ethyl-7-methyl-3H-imidazo[4,5-b]pyridine (PF-03247364)

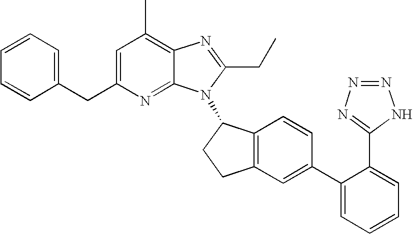

A solution of 5-benzyl-2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridine (0.479 g, 0.635 mmol) in acetone (6 mL) was stirred at RT. A solution of 3N HCl (2.12 mL, 6.35 mmol) was added in one portion. The mixture was stirred at RT overnight. Water was added (10 mL) and the mixture concentrated to an aqueous residue. Adjusted pH=13 with 2N KOH and solid filtered. The filtrate was extracted with ethyl ether (2×20 mL). No product found in ether extracts. Aqueous layer was cooled in an ice-bath and neutralized to pH=7.5 with 1M HCl. A cloudy solution formed. The mixture was extracted with EtOAc (3×50 ml) and brine (1×25 mL). The organic layers were combined, dried over magnesium sulfate, and concentrated to give 3-((1S)-5-(2-(1H-tetrazol-5-yl)phenyl)-2,3-dihydro-1H-inden-1-yl)-5-benzyl-2-ethyl-7-methyl-3H-imidazo[4,5-b]pyridine (PF-03247364) as a pale yellow solid (0.146 g, 45%): ¹H NMR (400 MHz, DMSO-d₆) δ ppm 1.27 (t, J=7.20 Hz, 3H), 2.45 (s, 3H), 2.52-2.59 (m, 1H), 2.61-2.73 (m, 2H), 2.82 (bs, 1H), 2.92-3.05 (m, 1H), 3.96 (bs, 2H), 6.30 (bs, 1H), 6.70-6.82 (m, 2H), 6.89 (s, 1H), 7.06-7.21 (m, 6H), 7.51-7.62 (m, 2H), 7.62-7.74 (m, 2H), CIMS: 512.2 (APCI)+, 510.2 (APCI)−; HPLC: 98.27% purity; Rt=11.854 min; method A.

### Example 2

### 3-((1S)-5-(2-(1H-tetrazol-5-yl)phenyl)-2,3-dihydro-1H-inden-1-yl)-2-ethyl-7-methyl-5-(pyridin-2-ylmethyl)-3H-imidazo[4,5-b]pyridine

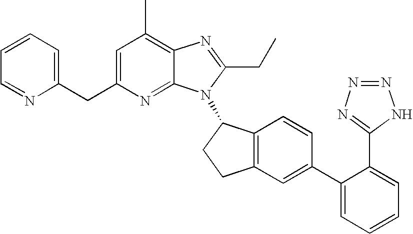

### Step 1 1-(pyridin-2-yl)pentane-2,4-dione

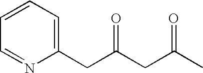

A suspension of sodium hydride 60% (in mineral oil, 8.8 g, 219.56 mmol) in THF (150 mL) was treated dropwise with 2,4-pentanedione (20 g, 199.64 mmol) in THF (100 mL) at 0° C. and for 20 min. n-Butyl lithium in hexane was added dropwise to the mixture (the solution turned into yellow gradually), and agitated at 0° C. for 30 min. 2-Fluoropyridine in THF (50 mL) was then added dropwise to the resultant mixture (the solution became red, and darker and darker), which was stirred overnight at room temperature. The reaction mixture was diluted with 300 mL of ether and then treated with 200 mL of brine. The pH was adjusted to 5 with 1 M hydrochloric acid at 0° C. The organic layer was isolated, the aqueous phase was extracted with ether (3×100 mL), the organic phases were combined, dried over sodium sulphate, filtered, and concentrated. The residue was purified via silica gel column chromatography to give 1-(pyridin-2-yl)pentane-2,4-dione (9.0 g, 25% yield) as a red oil. ¹H NMR (400 MHz, CDCl₃) δ: 8.58 (s, 1H), 7.63 (br s, 1H), 7.28 (d, 1H), 7.20 (s, 1H), 5.58 (s, 1H), 3.80 (s, 2H), 2.03 (s, 3H).

### Steps 2 and 3. 7-methyl-5-(pyridin-2-ylmethyl)-1H-imidazo[4,5-b]pyridin-2(3H)-one

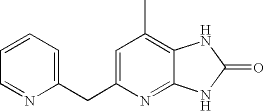

A solution of potassium hydroxide (2.66 g, 47.46 mmol) in methanol (50 mL) was treated with malonamamidine (5.46 g, 39.55 mmol) in small portions at 5-10° C., then stirred at 20° C. for 15 min. 1-(Pyridin-2-yl)pentane-2,4-dione (7.0 g, 39.55 mmol) in methanol (10 mL) was added dropwise to the mixture and was stirred at r.t. for 36 hr. Methanol (30 mL) was added to the reaction mixture of, followed by dropwise addition of potassium hydroxide (5.54 g, 98.87 mmol) in methanol (20 mL), and then agitated 30 min. The mixture was cooled to −10 to −5° C. and iodobenzene diacetate (12.73 g, 39.55 mmol) was added as a solid in portions over 20 min. The resultant mixture was aged at −10° C. for 3 hr, then allowed to warm up to r.t. overnight. The precipitated solid was filtered, washed with methanol to give a mixture of 7-methyl-5-(pyridin-2-ylmethyl)-1H-imidazo[4,5-b]pyridin-2(3H)-one and 5-methyl-7-(pyridin-2-ylmethyl)-1H-imidazo[4,5-b]pyridin-2(3H)-one (6.0 g, 63% yield). ¹H NMR (400 MHz, DMSO-d₆) δ: 11.18 (br s, 1H), 10.85 (br s, 1H), 8.41 (d, 1H), 7.65 (m, 1H), 7.23-7.20 (m, 2H), 6.72 (s, 1H), 4.08 (s, 2H), 2.03 (s, 3H); ESI-MS: 241.07.

### Step 4. 2-ethyl-7-methyl-5-(pyridin-2-ylmethyl)-3H-imidazo[4,5-b]pyridine

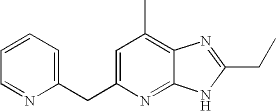

The mixture of 7-methyl-5-(pyridin-2-ylmethyl)-1H-imidazo[4,5-b]pyridin-2(3H)-one and 5-methyl-7-(pyridin-2-ylmethyl)-1H-imidazo[4,5-b]pyridin-2(3H)-one (3.0 g, 12.40 mmol) in concentrated hydrochloric acid (6 mL, 36-37%) was treated with propionic acid (3.71 mL, 49.6 mmol). The mixture was heated at 200° C. overnight in a sealed reactor. The reaction was cooled and solvent was removed in vacuo. The residue was suspended in water, pH was adjusted to 8 with ammonium hydroxide, and the mixture was extracted with dichloromethane (3×30 mL). The combined organic phase was dried over sodium sulphate, filtered, and concentrated. The residue was subjected to a silica gel column chromatography to give 2-ethyl-7-methyl-5-(pyridin-2-ylmethyl)-3H-imidazo[4,5-b]pyridine (1.40 g, not pure). ESI-MS: 215.11.

### Step 5. 2-ethyl-7-methyl-5-(pyridin-2-ylmethyl)-3-((1S)-5-(2-(1-trityl-1H-tetrazol-5-yl)phenyl)-2,3-dihydro-1H-inden-1-yl)-3H-imidazo[4,5-b]pyridine

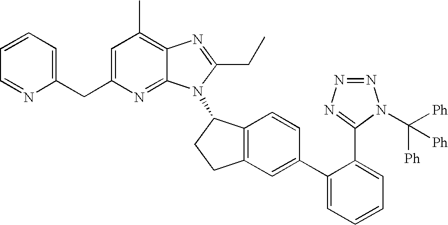

A solution of 2-ethyl-7-methyl-5-(pyridin-2-ylmethyl)-3H-imidazo[4,5-b]pyridine (1.75 g, 6.94 mmol), (1R)-5-(2-(1-trityl-1H-tetrazol-5-yl)phenyl)-2,3-dihydro-1H-inden-1-ol (5.42 g, 10.42 mmol), and triphenylphosphine (5.48 g 20.83 mmol) in dry THF (80 mL) was treated with diethylazodicarboxylate (2.73 mL, 17.36 mmol) and diisopropylethylamine (1.82 mL, 10.42 mmol) at 0° C. The reaction was stirred at room temperature for 42 hr. The reaction mixture was concentrated to a dark red residue, which was purified via silica gel column chromatography to give 2-ethyl-7-methyl-5-(pyridin-2-ylmethyl)-3-((1S)-5-(2-(1-trityl-1H-tetrazol-5-yl)phenyl)-2,3-dihydro-1H-inden-1-yl)-3H-imidazo[4,5-b]pyridine (2.75 g 53% yield). ESI-MS: 755.48.

### Step 6. 3-((1S)-5-(2-(1H-tetrazol-5-yl)phenyl)-2,3-dihydro-1H-inden-1-yl)-2-ethyl-7-methyl-5-(pyridin-2-ylmethyl)-3H-imidazo[4,5-b]pyridine

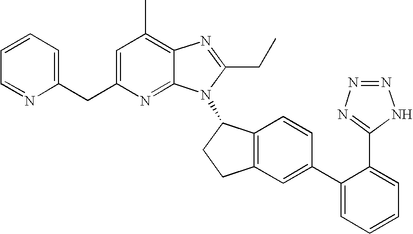

A solution of 2-ethyl-7-methyl-5-(pyridin-2-ylmethyl)-3-((1S)-5-(2-(1-trityl-1H-tetrazol-5-yl)phenyl)-2,3-dihydro-1H-inden-1-yl)-3H-imidazo[4,5-b]pyridine (2.75 g, 3.64 mmol) in methanol (30 mL) was heated at reflux overnight. The solvent was removed in vacuo. The residue was subjected to a silica gel column chromatography to give 3-((1S)-5-(2-(1H-tetrazol-5-yl)phenyl)-2,3-dihydro-1H-inden-1-yl)-2-ethyl-7-methyl-5-(pyridin-2-ylmethyl)-3H-imidazo[4,5-b]pyridine (1.05 g, 54% yield). ¹H NMR (400 MHz, CDCl₃) δ ppm 7.95 (d, 1H), 7.90 (d, 1H), 7.65 (m, 2H), 7.58 (d, 2H), 7.05 (m, 3H), 6.95 (s, 1H), 6.50 (m, 2H), 5.80 (m, 1H), 4.65 (d, 1H), 4.30 (d, 1H), 3.01 (m, 2H), 2.80 (m, 1H), 2.61 (s, 3H), 2.55 (m, 2H), 2.22 (m, 1H), 1.45 (t, 3H); ESI-MS: 513.29; HPLC: 97.68%; Elemental analysis for C₃₁H₂₈N₈.0.8H₂O: C, 70.65; H, 5.66; N, 21.26; Found: C, 70.50; H, 5.47; N, 20.84.

### Example 3

### 3-((1S)-5-(2-(1H-tetrazol-5-yl)phenyl)-2,3-dihydro-1H-inden-1-yl)-2-ethyl-5-(methoxymethyl)-7-methyl-3H-imidazo[4,5-b]pyridine (reference: 05-001-190)

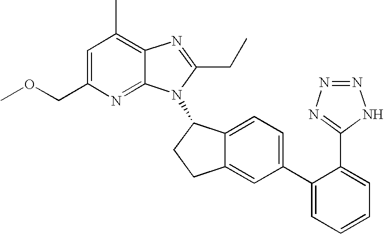

### Step 1. (S)-3-(5-bromo-2,3-dihydro-1H-inden-1-yl)-2-ethyl-5-(methoxymethyl)-7-methyl-3H-imidazo[4,5-b]pyridine

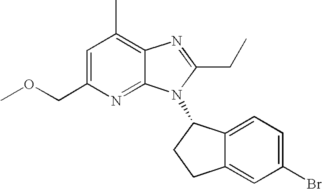

A solution of (S)-(3-(5-bromo-2,3-dihydro-1H-inden-1-yl)-2-ethyl-7-methyl-3H-imidazo[4,5-b]pyridin-5-yl)methanol (0.60 g, 1.55 mmol) in THF was treated with sodium hydride (60%, 0.08 g, 2.02 mmol) and stirred at 0° C. for 30 min. The reaction mixture was treated with methyl iodide (0.12 mL, 1.90 mmol) and stirred at room temperature overnight. The reaction mixture was quenched with saturated ammonium chloride solution (40 mL). The product was extracted with ethyl acetate (2×25 mL) and the solvent was removed. The crude product was purified via silica gel column chromatography to give (S)-3-(5-bromo-2,3-dihydro-1H-inden-1-yl)-2-ethyl-5-(methoxymethyl)-7-methyl-3H-imidazo[4,5-b]pyridine (0.28 g, 45%). ¹H NMR (400 MHz, CDCl₃) δ 7.48 (s, 1H), 7.27-7.24 (m, 1H), 7.13 (s, 1H), 6.74 (d, 1H), 6.44 (br s, 1H), 4.54 (s, 2H), 3.42-2.50 (m, 12H), 1.33 (t, 3H). MS=402 (M+).

### Step 2. 2-ethyl-5-(methoxymethyl)-7-methyl-3-((1S)-5-(2-(1-trityl-1H-tetrazol-5-yl)phenyl)-2,3-dihydro-1H-inden-1-yl)-3H-imidazo[4,5-b]pyridine

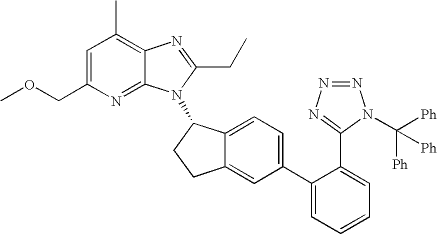

To a degassed solution of PPh₃ (0.073 g, 0.30 mmol) in DME (15 mL) was added Pd(OAc)₂ (0.016 g, 0.07 mmol) and stirred for 10 min. To the reaction mixture was added 2-(1-trityl-1H-tetrazol-5-yl)phenylboronic acid (0.38 g, 0.84 mmol), K₂CO₃ (0.24 g, 1.80 mmol), (S)-3-(5-bromo-2,3-dihydro-1H-inden-1-yl)-2-ethyl-5-(methoxymethyl)-7-methyl-3H-imidazo[4,5-b]pyridine (0.28 g, 0.70 mmol) and water (0.03 mL, 1.80 mmol). The reaction mixture was heated at 90° C. overnight in a sealed tube. The solvent was removed under vacuum and the residue was purified via silica gel column chromatography to afford 2-ethyl-5-(methoxymethyl)-7-methyl-3-((S)-5-(2-(1-trityl-1H-tetrazol-5-yl)phenyl)-2,3-dihydro-1H-inden-1-yl)-3H-imidazo[4,5-b]pyridine (0.32 g, 66%). MS=708 (M+).

### Step 3. 3-((1S)-5-(2-(1H-tetrazol-5-yl)phenyl)-2,3-dihydro-1H-inden-1-yl)-2-ethyl-5-(methoxymethyl)-7-methyl-3H-imidazo[4,5-b]pyridine

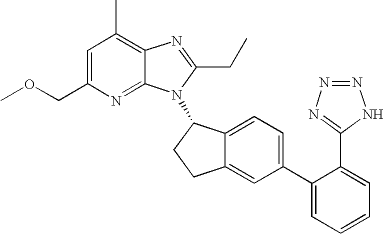

A solution of 2-ethyl-5-(methoxymethyl)-7-methyl-3-((S)-5-(2-(1-trityl-1H-tetrazol-5-yl)phenyl)-2,3-dihydro-1H-inden-1-yl)-3H-imidazo[4,5-b]pyridine (0.32 g, 0.45 mmol) in methanol (10 mL) was heated at reflux overnight. The solvent was removed under vacuum. The compound was precipitated from a mixture of dichloromethane, ether and hexane to afford 3-((1S)-5-(2-(1H-tetrazol-5-yl)phenyl)-2,3-dihydro-1H-inden-1-yl)-2-ethyl-5-(methoxymethyl)-7-methyl-3H-imidazo[4,5-b]pyridine (0.175 g, 83%). ¹H NMR (400 MHz, CDCl₃) δ 8.20 (d, 1H), 7.60-6.80 (m, 7H), 4.60-4.50 (m, 2H), 3.40-2.58 (m, 14H), 1.45 (br s, 3H) MS=466.02 (M+), HPLC: 95.53%. Calculated for C₂₇H₂₇N₇O.0.3CH₂Cl₂: C, 66.78%; H, 5.67%; N, 19.97%; Found: C, 66.88%; H, 5.86%; N, 18.75%.

### Example 4

### 3-((1S)-5-(2-(1H-tetrazol-5-yl)phenyl)-2,3-dihydro-1H-inden-1-yl)-2-ethyl-5-(isopropoxymethyl)-7-methyl-3H-imidazo[4,5-b]pyridine

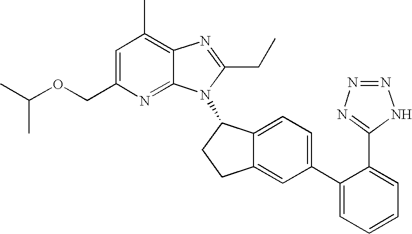

### Step 1. (S)-(3-(5-bromo-2,3-dihydro-1H-inden-1-yl)-2-ethyl-7-methyl-3H-imidazo[4,5-b]pyridin-5-yl)methyl methanesulfonate

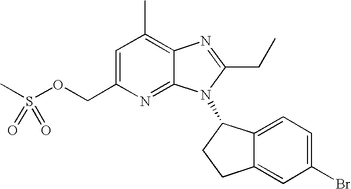

A solution of (S)-(3-(5-bromo-2,3-dihydro-1H-inden-1-yl)-2-ethyl-7-methyl-3H-imidazo[4,5-b]pyridin-5-yl)methanol (0.25 g, 0.65 mmol) in dichloromethane (25 mL) was treated with triethylamine (0.28 mL, 1.94 mmol) followed by dropwise addition of methanesulfonylchloride (0.06 mL, 0.78 mmol) at 0° C. The reaction mixture was stirred at 0° C. for 2 h. The reaction was quenched with water (30 mL). The organic layer was separated, washed with brine, dried over anhydrous sodium sulfate and evaporated to give (S)-(3-(5-bromo-2,3-dihydro-1H-inden-1-yl)-2-ethyl-7-methyl-3H-imidazo[4,5-b]pyridin-5-yl)methyl methanesulfonate as a yellow foamy solid. This was used for next step without further purification.

### Step 2. (S)-3-(5-bromo-2,3-dihydro-1H-inden-1-yl)-2-ethyl-5-(isopropoxymethyl)-7-methyl-3H-imidazo[4,5-b]pyridine

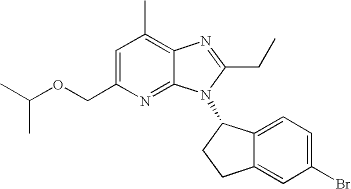

A solution of (S)-(3-(5-bromo-2,3-dihydro-1H-inden-1-yl)-2-ethyl-7-methyl-3H-imidazo[4,5-b]pyridin-5-yl)methyl methanesulfonate (0.65 mmol) in isopropanol (10 mL) was treated with potassium isopropoxide solution in isopropanol (2.00 mL, 5% w/v solution) at 0° C. The reaction mixture was stirred at room temperature for 2d. The reaction mixture was quenched with saturated ammonium chloride solution (20 mL). The product was extracted with ethyl acetate (2×25 mL) and the solvent was removed. The crude product was purified using column chromatography to give (S)-3-(5-bromo-2,3-dihydro-1H-inden-1-yl)-2-ethyl-5-(isopropoxymethyl)-7-methyl-3H-imidazo[4,5-b]pyridine (0.18 g, 66%). ¹H NMR (400 MHz, CDCl₃) δ 7.42 (s, 1H), 7.214 (d, 1H), 7.15 (s, 1H), 6.74 (d, 1H), 6.44 (br s, 1H), 4.58 (s, 2H), 3.70-3.60 (m, 1H), 3.35-2.40 (m, 9H), 1.38 (t, 3H), 1.20 (t, 6H). MS=430 (M+).

### Step 3. 2-ethyl-5-(isopropoxymethyl)-7-methyl-3-((S)-5-(2-(1-trityl-1H-tetrazol-5-yl)phenyl)-2,3-dihydro-1H-inden-1-yl)-3H-imidazo[4,5-b]pyridine

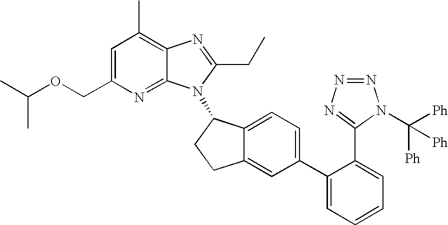

To a degassed solution of PPh₃ (0.044 g, 0.17 mmol), 2-(1-trityl-1H-tetrazol-5-yl)phenylboronic acid (0.23 g, 0.51 mmol) and (S)-3-(5-bromo-2,3-dihydro-1H-inden-1-yl)-2-ethyl-5-(isopropoxymethyl)-7-methyl-3H-imidazo[4,5-b]pyridine (0.18 g, 0.42 mmol) in DME (15 mL) were added water (1 drop), Pd(OAc)₂ (0.009 g, 0.04 mmol) and K₂CO₃ (0.15 g, 1.05 mmol), The reaction mixture was heated at 90° C. overnight in a sealed tube. The solvent was removed under vacuum and the residue was subjected to column chromatography to afford 2-ethyl-5-(isopropoxymethyl)-7-methyl-3-((S)-5-(2-(1-trityl-1H-tetrazol-5-yl)phenyl)-2,3-dihydro-1H-inden-1-yl)-3H-imidazo[4,5-b]pyridine (0.24 g, 77%).

MS=736 (M+).

### Step 4. 3-((1S)-5-(2-(1H-tetrazol-5-yl)phenyl)-2,3-dihydro-1H-inden-1-yl)-2-ethyl-5-(isopropoxymethyl)-7-methyl-3H-imidazo[4,5-b]pyridine

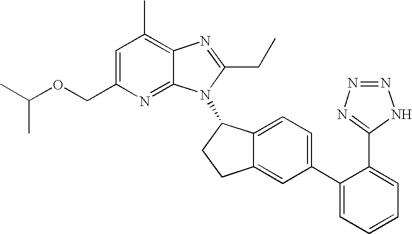

A solution of 2-ethyl-5-(isopropoxymethyl)-7-methyl-3-((S)-5-(2-(1-trityl-1H-tetrazol-5-yl)phenyl)-2,3-dihydro-1H-inden-1-yl)-3H-imidazo[4,5-b]pyridine (0.25 g, 0.34 mmol) in methanol (10 mL) was heated at reflux overnight. The solvent was removed under vacuum and the mixture treated with dichloromethane, ether and hexane to afford 3-((1S)-5-(2-(1H-tetrazol-5-yl)phenyl)-2,3-dihydro-1H-inden-1-yl)-2-ethyl-5-(isopropoxymethyl)-7-methyl-3H-imidazo[4,5-b]pyridine (0.11 g, 66%) as a precipitate. 1H NMR (400 MHz, CHLOROFORM-d) δ ppm 1.18 (d, J=5.47 Hz, 3H) 1.21-1.30 (m, 6H) 2.80-2.88 (m, 3H) 2.92 (br. s., 2H) 3.08-3.31 (m, 2H) 3.32-3.52 (m, 2H) 3.67-3.83 (m, 2H) 4.55-4.78 (m, 3H) 6.68 (br. s., 1H) 6.88 (t, J=6.83 Hz, 1H) 6.93-7.00 (m, 1H) 7.29 (s, 1H) 7.36-7.78 (m, 2H) 7.99-8.14 (m, 1H). MS=494 (M+), HPLC: 93.30%. Calculated for C₂₉H₃₁N₇O.0.5H₂O: C, 69.30%; H, 6.42%; N, 19.67%; Found: C, 69.21%; H, 6.62%; N, 17.87%.

### Example 5

### 3-((1S)-5-(2-(1H-tetrazol-5-yl)phenyl)-2,3-dihydro-1H-inden-1-yl)-5-(tert-butoxymethyl)-2-ethyl-7-methyl-3H-imidazo[4,5-b]pyridine

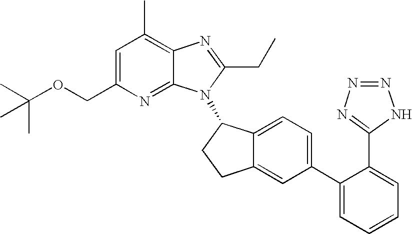

3-((1S)-5-(2-(1H-tetrazol-5-yl)phenyl)-2,3-dihydro-1H-inden-1-yl)-5-(tert-butoxymethyl)-2-ethyl-7-methyl-3H-imidazo[4,5-b]pyridine was prepared via analogous procedures as Example 4 except that in Step 2 potassium tert-butoxide in t-butyl alcohol was used to displace the mesylate.

### Example 6

### 5-((1H-pyrazol-1-yl)methyl)-3-((1S)-5-(2-(1H-tetrazol-5-yl)phenyl)-2,3-dihydro-1H-inden-1-yl)-2-ethyl-7-methyl-3H-imidazo[4,5-b]pyridine

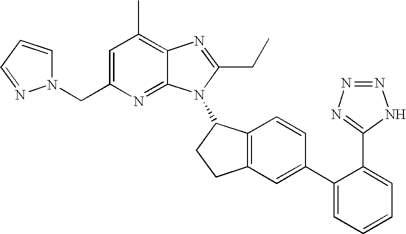

5-((1H-pyrazol-1-yl)methyl)-3-((1S)-5-(2-(1H-tetrazol-5-yl)phenyl)-2,3-dihydro-1H-inden-1-yl)-2-ethyl-7-methyl-3H-imidazo[4,5-b]pyridine was prepared from analogous procedures as Example 4 except in Step 2 the mesylate was substituted with pyrazole anion. 1H NMR (400 MHz, CHLOROFORM-d) δ ppm 1.42 (t, J=7.61 Hz, 3H) 2.28-2.45 (m, 1H) 2.53 (s, 1H) 2.60 (s, 3H) 2.89 (s, J=8.00, 8.00 Hz, 2H) 3.05 (s, J=7.03 Hz, 2H) 5.31 (d, J=16.40 Hz, 1H) 5.86 (d, J=16.40 Hz, 2H) 6.26 (s, 1H) 6.48 (q, 2H) 6.84 (s, 1H) 7.24-7.32 (m, J=5.86 Hz, 1H) 7.45-7.54 (m, 2H) 7.55-7.63 (m, 1H) 7.87 (dd, J=8.00, 1.37 Hz, 1H).

### Example 7

### (3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)(phenyl)methanol

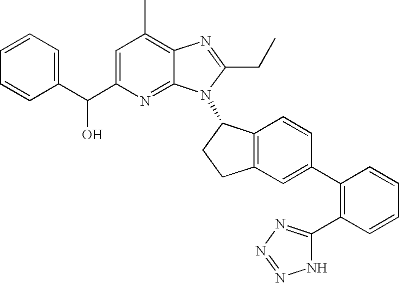

### Step 1: (3-((S)-5-bromo-2,3-dihydro-1H-inden-1-yl)-2-ethyl-7-methyl-3H-imidazo[4,5-b]pyridin-5-yl)(phenyl)methanol

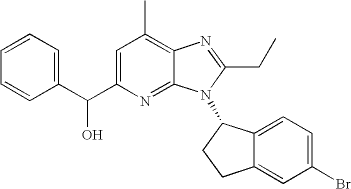

To a solution of 3 N phenylmagnesium bromide (1.739 mL, 5.216 mmol) in THF (30 mL) at −78° C. under N₂ atmosphere was added a solution of (S)-3-(5-bromo-2,3-dihydro-1H-inden-1-yl)-2-ethyl-7-methyl-3H-imidazo[4,5-b]pyridine-5-carbaldehyde (1.002 g, 2.608 mmol)) in THF (10 mL) and the reaction stirred for 1 hour at −78° C. LCMS indicated complete conversion. Reaction quenched with sat. NH₄Cl solution and filtered through celite. Filtrate solvent removed in vacuo and residue partitioned between Et₂O (40 mL) and water (20 mL) and layers separated. Organic layer washed with brine (20 mL) and dried over MgSO₄. LCMS before chromatography showed 3:1 ratio of diastereomers. Residue chromatographed with 50% EtOAc/Hexanes to give (3-((S)-5-bromo-2,3-dihydro-1H-inden-1-yl)-2-ethyl-7-methyl-3H-imidazo[4,5-b]pyridin-5-yl)(phenyl)methanol (0.945 g) as a mixture of diastereomers. MS: 463.2 (APCI)⁺.

### Step 2: (2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)(phenyl)methanol

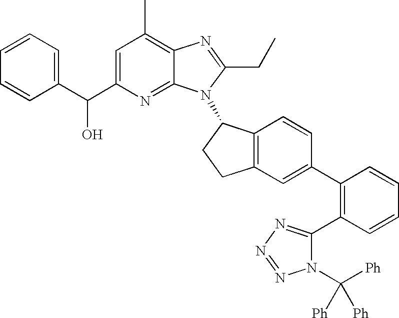

To a solution of triphenylphosphine (0.115 g, 0.437 mmol) in degassed DME (20 mL) (15 min bubbling) was added Pd(OAc)₂ (0.024 g, 0.107 mmol) and solution turned bright yellow. After stirring for an additional 15 minutes, potassium carbonate (0.442 g, 3.199 mmol), (2-(2-trityl-imidazole)-phenyl boronic acid (0.691 g, 1.599 mmol) and (3-((S)-5-bromo-2,3-dihydro-1H-inden-1-yl)-2-ethyl-7-methyl-3H-imidazo[4,5-b]pyridin-5-yl)(phenyl)methanol (0.493 g, 1.066 mmol) followed by water (0.230 mL, 12.794 mmol) were added and the solution degassed for an additional 10 minutes. The suspension was then heated to 100° C. for 16 hours. Solvent removed in vacuo and residue chromatographed to give (2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)(phenyl)methanol (0.720 g) as a mixture of diastereomers. MS: 770.5, 528.3 (loss of trityl group) (APCI)⁺. 526.5 (APCI)⁻.

### Step 3: (3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)(phenyl)methanol

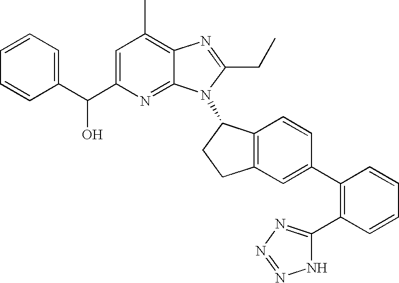

A solution of (2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)(phenyl)methanol (0.720 g, 0.935 mmol) in MeOH (20 mL) was heated at 80° C. for 4 hours. Solvent removed in vacuo to give (3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)(phenyl)methanol.

### Example 8a

### (S)-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)(phenyl)methanol

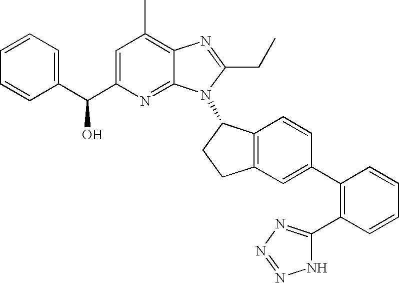

### Example 8b

### (R)-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)(phenyl)methanol

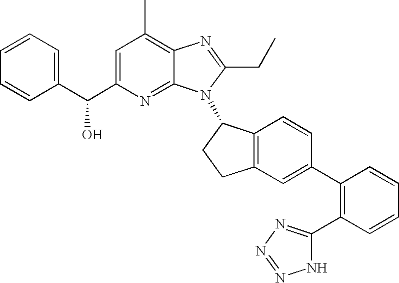

(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)(phenyl)methanol was chromatographed on silica gel with 100% EtOAc to give 8a (27 mg) as a single diastereomer 1H NMR (400 MHz, CHLOROFORM-d) δ ppm 1.55 (t, J=7.60 Hz, 3H) 2.43-2.54 (m, 1H) 2.59 (s, 3H) 2.86-3.03 (m, 1H) 3.17 (s, 2H) 3.27-3.40 (m, 1H) 3.50-3.65 (m, 1H) 5.90 (s, 1H) 5.93 (s, 1H) 6.08 (dd, J=8.19, 3.90 Hz, 1H) 6.62 (d, J=7.80 Hz, 1H) 6.80 (s, 1H) 6.88 (d, J=7.80 Hz, 1H) 7.22-7.37 (m, 5H) 7.50-7.56 (m, J=7.02 Hz, 2H) 7.61 (t, J=7.41 Hz, 1H) 7.68 (s, 1H) 7.89 (d, J=7.80 Hz, 1H). MS: 528.3 (APCI)⁺, 526.2 (APCI)⁻. 8b (105 mg) as a second diastereomer was also isolated. 1H NMR (400 MHz, CHLOROFORM-d) δ ppm 1.56 (t, J=7.12 Hz, 3H) 2.36 (s, 1H) 2.42 (s, 1H) 2.58 (s, 3H) 2.75-2.91 (m, 1H) 3.02-3.13 (m, 1H) 3.14-3.32 (m, J=23.00 Hz, 3H) 5.70 (s, 1H) 6.14 (s, 1H) 6.62 (s, 1H) 7.08-7.21 (m, J=19.30 Hz, 3H) 7.22-7.36 (m, 5H) 7.48-7.56 (m, 2H) 7.57-7.64 (m, 1H) 7.90 (d, J=7.41 Hz, 1H).

### Example 9

### 3-((1S)-5-(2-(1H-tetrazol-5-yl)phenyl)-2,3-dihydro-1H-inden-1-yl)-5-allyl-2-ethyl-7-methyl-3H-imidazo[4,5-b]pyridine

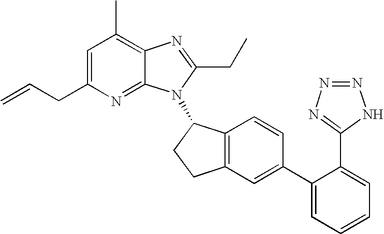

### Step 1. N-(6-Allyl-2-chloro-methyl-pyridin-3-yl)-propionamide

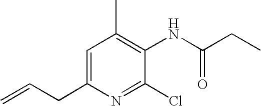

A mixture of N-(6-bromo-2-chloro-methyl-pyridin-3-yl)-propionamide (15 g, 54 mmol) and LiBr (235 mg, 2.7 mmol) in dry THF (55 mL) was stirred under nitrogen at RT. A solution of allylmagnesium bromide (1M in ether, 122 mL) was added slowly. The reaction mixture was heated under refluxed for 2 h. After cooling, an aqueous solution of NH₄Cl (50 mL) and ethyl acetate (100 mL) were added. The aqueous layer was extracted with ethyl acetate (2×50 mL), the combined organic extracts were evaporated. The crude product was purified by MPLC on a silica gel column using a step gradient of ethyl acetate in hexanes of 15-75%. Pure fractions were combined and solvent evaporated to give N-(6-Allyl-2-chloro-methyl-pyridin-3-yl)-propionamide (13 g, 83%). MS: 237, 239 (3:1) (APCI)⁻ 1H NMR (400 MHz, CDCl3) δ ppm 7.19 (s, 1H), 7.02 (s, 1H), 5.95 (m, 2H), 5.14 (m, 2H), 3.52 (d, 2H), 2.45 (q, 2H), 2.26 (s, 3H), 1.26 (t, 3H).

### Step 2. N-(6-Allyl-2-chloro-methyl-pyridin-3-yl)-N′—((S)-5-bromo-indan-1-yl)-propionamide

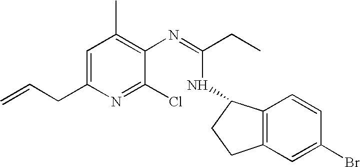

A mixture of N-(6-Allyl-2-chloro-methyl-pyridin-3-yl)-propionamide (3.45 g, 14.5 mmol) and PCl₅ (3.2 g, 15.2 mmol) in 10 mL of DCM was refluxed for 3 h. Solvent was removed under vacuo and pumped to dryness. A solution of the intermediate imidoylchloride in anhydrous DCM (15 mL) was added at RT into a solution of (S)-5-bromo-indan-1-ylamine (4 g, 17.3 mmol) and triethylamine (4 g, 36.1 mmol) in 15 mL of DCM at 0° C. The reaction mixture was stirred at RT overnight, and then filtered through a short packed silica gel and eluted with ether. The crude product was purified by MPLC on a silica gel column using a step gradient of ethyl acetate in hexanes of 15-75%. Pure fractions were combined and solvent evaporated to give N-(6-Allyl-2-chloro-methyl-pyridin-3-yl)-N′—((S)-5-bromo-indan-1-yl)-propionamide (5.54 g, 88.7%).

MS: 432, 434, 436 (APCI)⁺

### Step 3. 5-allyl-2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridine)

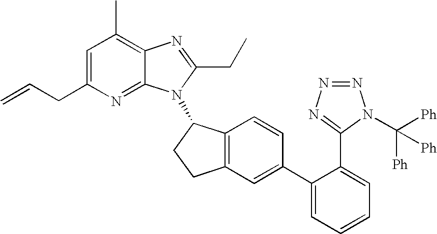

Catalytic palladium (II) acetate (0.3 g, 1.27 mmol) was added to a solution of triphenylphosphine (1.33 g, 5 mmol), 2-(1-trityl-1H-tetrazol-5-yl)-phenyl boronic acid (5.77 g, 13.3 mmol), K₂CO₃ (7.03 g, 50.8 mmol) and N-(6-Allyl-2-chloro-methyl-pyridin-3-yl)-N′—((S)-5-bromo-indan-1-yl)-propionamide (5.5 g, 13 mmol) in dry DME (15 mL). The mixture was stirred at room temperature for 10 minutes with bubbling N₂, and water (1.37 mL, 76.2 mmol) was added. The stirred reaction mixture was treated with bubbling N₂ for an additional 20 minutes, and then heated at 85° C. for approximately 3 hours. S-Phos (2-dicyclohexylphosphino-2′,6′-dimethoxy-1,1′-biphenyl) (1.04 g, 2.54 mmol) and palladium (II) acetate (0.15 g, 0.63 mmol) was added to the reaction mixtures, then heated at 85° C. for approximately 18 hours. The mixture was diluted with EtOAc and the resulting solution was filtered through a Celite 521 and the initial filtrate and EtOAc washings were combined. The solvents were removed, and the residue was purified by MPLC on a silica gel column using a step gradient of ethyl acetate in hexanes (15-75%). Pure fractions were combined and evaporated to give 5-allyl-2-ethyl-7-methyl-3-{(S)-5-[2-(1-trityl-1H-tetrazol-5-yl)-phenyl]-indan-1-yl}-3H-imidazo[4,5-b]pyridine as a color foam (4.1 g, 46%). MS: 704.4 (APCI)⁺.

### Step 4. 3-((1S)-5-(2-(1H-tetrazol-5-yl)phenyl)-2,3-dihydro-1H-inden-1-yl)-5-allyl-2-ethyl-7-methyl-3H-imidazo[4,5-b]pyridine

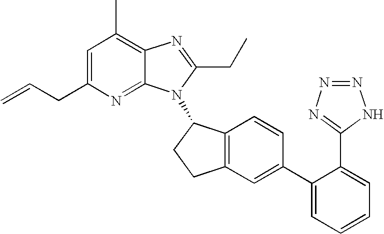

A solution of 5-Allyl-2-ethyl-7-methyl-3-{(S)-5-[2-(1-trityl-1H-tetrazol-5-yl)-phenyl]-indan-1-yl}-3H-imidazo[4,5-b]pyridine (0.34 g, 0.483 mmol) in methanol (10 mL), was refluxed under nitrogen for 3 hours. The solvent was removed under vacuo. The residue was purified by MPLC on a silica gel column using a step gradient of MeOH in DCM (1-5%). Pure fractions were combined and evaporated to afford 5-allyl-2-ethyl-7-methyl-3-{(S)-5-[2-(1H-tetrazol-5-yl)-phenyl]-indan-1-yl}-3H-imidazo[4,5-b]pyridine (0.71 g, 76%). MS: 462.3 (APCI)⁺; 460.3 (APCI)⁻ HPLC showed >88% purity. Retention time=11.74 minutes; method 90 to 10% 20 minutes 254 nM (detection wavelength).

### Example 10

### (S)-1-(2-Ethyl-7-methyl-3-{(S)-5-[2-(1H-tetrazol-5-yl)-phenyl]-indan-1-yl}-3H-imidazo[4,5-b]pyridin-5-yl)-2-methyl-propan-1-ol

### [(S)-1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-2-methylpropan-1-ol]

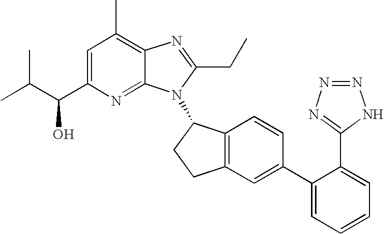

### Steps 1 and 2. (S)-methyl 3-(5-bromo-2,3-dihydro-1H-inden-1-yl)-2-ethyl-7-methyl-3H-imidazo[4,5-b]pyridine-5-carboxylate

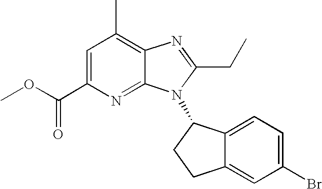

A cooled (0° C.) mixture of 5-bromo-2-ethyl-7-methyl-imidazo[4,5-b]pyridine (3.4 g, 15.18 mmol), (R)-5-bromo-indan-1-ol (3.86 g, 18.21 mmol) and Bu₃P (9.64 mL, 38.0 mmol) in toluene (30 mL) was treated dropwise with diethylazodicarboxylate (DEAD, 4.82 mL, 30.36 mmol). After the addition was complete, the mixture was allowed to warm up to 23° C. and stirred at 23° C. for 1 h, followed by addition of di-isopropylethylamine (4.44 mL). The mixture thus obtained was stirred at 70° C. for 16 h, cooled to 23° C., concentrated, and purified by silica gel chromatography to give 5-bromo-3-(5-bromo-indan-1-yl)-2-ethyl-7-methyl-3H-imidazo[4,5-b]pyridine. The crude residue was mixed with Pd(PPh₃)₂Cl₂ (0.53 g, 0.76 mmol), Et₃N (4 mL) and MeOH (40 mL). The mixture was heated at 70° C. under CO atmosphere for 180 h, cooled to 23° C., concentrated. The residue was purified by chromatography on silica gel to give 3-(5-bromo-indan-1-yl)-2-ethyl-7-methyl-3H-imidazo[4,5-b]pyridine-5-carboxylic acid methyl ester (3.43 g, 52% in two steps) as a pale yellow solid. ¹HNMR (400 MHz, CDCl₃) δ ppm: 7.97 (s, 1H), 7.50 (s, 1H), 7.25 (d, 1H), 6.80 (d, 1H), 6.60 (m, 1H), 4.00 (s, 3H), 3.35 (m, 1H), 3.10 (m, 1H), 2.85 (m, 1H), 2.73 (s, 3H), 2.63 (m, 1H), 2.50 (m, 2H), 1.30 (t, 3H).

### Step 3: (S)-(3-(5-bromo-2,3-dihydro-1H-inden-1-yl)-2-ethyl-7-methyl-3H-imidazo[4,5-b]pyridin-5-yl)methanol

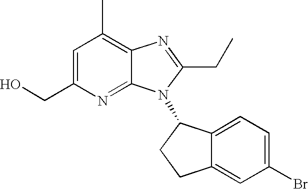

A cooled (0° C.) solution of 3-(5-bromo-indan-1-yl)-2-ethyl-7-methyl-3H-imidazo[4,5-b]pyridine-5-carboxylic acid methyl ester (3.43 g, 8.29 mmol) in THF was treated with lithium aluminum hydride (10.0 mL, 1 M in THF). The mixture was stirred at 0° C. for 30 min, EtOAc (5 mL) and aq. NH₄Cl (2 mL) were added subsequently. The mixture was stirred for 5 min, dried over Na₂SO₄ (20 g), and concentrated to give (S)-(3-(5-bromo-2,3-dihydro-1H-inden-1-yl)-2-ethyl-7-methyl-3H-imidazo[4,5-b]pyridin-5-yl)methanol (3.20 g). This was used in the next step without further purification. ¹H-NMR (400 MHz, CDCl₃) δ ppm 7.52 (s, 1H), 7.24 (d, 1H), 6.90 (s, 1H), 6.75 (d, 1H), 6.30 (m, 1H), 4.70 (s, 2H), 3.40 (m, 2H), 3.10 (m, 1H), 2.90-2.40 (m, 6H), 1.30 (m, 3H).

### Step 4: (S)-1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-2-methylpropan-1-ol

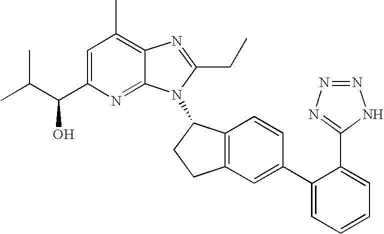

A cooled (−78° C.) solution of oxalyl chloride (10.4 mL, 2 M in dichloromethane) in dichloromethane (20 mL) was treated with dimethylsulfoxide (2.94 mL, 51.15 mmol). The mixture was stirred at −78° C. for 15 min. A solution of [3-(5-bromo-indan-1-yl)-2-ethyl-7-methyl-3H-imidazo[4,5-b]pyridin-5-yl]-methanol (3.20 g, 8.29 mmol) in dichloromethane (20 mL+10 mL rinse) was added. The mixture was stirred at −78° C. for 1 h. Triethylamine (8.7 mL, 62.18 mmol) was added, then allowed to stir at −78° C. for 15 min, then warmed up to RT, quenched with H₂O, taken into dichloromethane (200 mL), washed with water (25 mL), dried and concentrated. The crude aldehyde was then dissolved in ether (100 mL) and cooled to −78° C. To this solution was added isopropyl magnesium chloride (8.29 mL, 2 M in ether). The mixture was stirred at −78° C. for 3 h, quenched with H₂O, taken into dichloromethane (200 mL), washed with H₂O, dried and concentrated. A mixture of crude alcohol, (2-(2-trityl-imidazole)-phenyl boronic acid (5.62 g, 13.26 mmol), Pd(OAc)₂ (371 mg, 1.66 mmol), PPh₃ (1.73 g, 6.61 mmol) and K₂CO₃ (2.86 g, 20.73 mmol) in DME—H₂O (30 mL-0.3 mL) was degassed for 10 min with N₂. The resulting mixture was heated at 90° C. in a sealed tube for 16 h, cooled to RT, concentrated and purified by silica gel chromatography to give the coupling product which was heated in methanol (5 mL) for 16 h. After concentration, the residue was purified by chromatography to give Example 10 (S)-1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-2-methylpropan-1-ol (580 mg) and Example 11 (R)-1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-2-methylpropan-1-ol (510 mg).

Data for 10 (S)-1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-2-methylpropan-1-ol, ¹HNMR (400 MHz, CDCl₃) δ ppm 8.05 (d, 1H), 7.65-7.50 (m, 3H), 7.25 (m, 1H), 7.10 (d, 1H), 6.95 (s, 1H), 6.80 (s, 1H), 6.05 (m, 1H), 3.20-2.70 (m, 4H), 2.65 (s, 3H), 2.30 (m, 1H), 2.05 (m, 1H), 1.30 (m, 3H), 1.00 (d, 3H), 0.40 (d, 3H). MS: 494 (M⁺+1). HPLC: 98.35%. Elemental Analysis calculated for C₂₉H₃₁N₇O.⅔H₂O: C, 68.89; H, 6.45; N, 19.38. Found: C, 68.84; H, 6.36; N, 18.55.

### Example 10b

### Alternative synthetic route to (S)-1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-2-methylpropan-1-ol

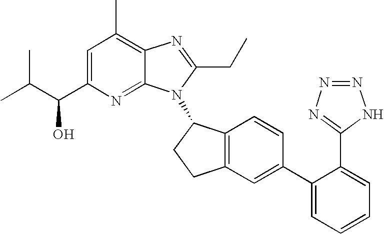

### Step 1. 6-Bromo-2-chloro-4-methyl-pyridin-3-ylamine

### [6-bromo-2-chloro-4-methylpyridin-3-amine]

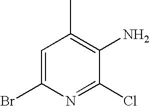

3-Amino-2-chloro-4-methylpyridine (50.0 g, 351 mmol) was dissolved in dichloromethane (500 mL) under nitrogen and the solution cooled in an ice bath to between 0-1° C. Dibromo-5,5-dimethylhydantoin (51.1 g, 179 mmol) was added in 5 portions over 40 min. After the addition was finished, the mixture was stirred at room temperature for 2 hours. The reaction mixture was passed through a short pad of silica gel, eluted with 30% ether/dichloromethane (1 L). The mixture was concentrated and heptane was added. The precipitate was collected by filtration, washed with heptane, and air dried to give 6-bromo-2-chloro-4-methyl-pyridin-3-ylamine (73.3 g, 94%). MS: 223.0 (APCI)⁺; 220.9 (APCI)⁻

### Step 2. N-(6-Bromo-2-chloro-4-methyl-pyridin-3-yl)-propionamide

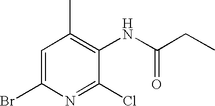

A solution of 6-bromo-2-chloro-4-methyl-pyridin-3-ylamine (73.3 g, 331 mmol) in toluene (100 mL) was treated with propionic anhydride (44.5 mL, 347 mmol). The mixture was heated under reflux overnight under nitrogen. The mixture was cooled to 70° C. and then heptane added. Allowed to stir and cool to 23° C. The precipitate was collected by filtration, washed with heptane, and air dried to give N-(6-Bromo-2-chloro-4-methyl-pyridin-3-yl)-propionamide. (86.4 g, 94%). MS: 279.0 (APCI)⁺; 277.0 (APCI)⁻

### Step 3. N-(2-Chloro-6-cyano-4-methyl-pyridin-3-yl)-propionamide

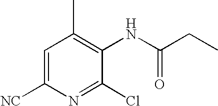

N-(6-Bromo-2-chloro-4-methyl-pyridin-3-yl)-propionamide (255 g, 919 mmol) was dissolved in dry DMF (250 mL) and the solution degassed by bubbling nitrogen through the solution for 10 min. Zinc cyanide (55.0 g, 469 mmol) and Pd(PPh₃)₄ (31.9 g, 27.6 mmol) were added to reaction mixture under a nitrogen blanket. The mixture was heated to 80° C. for 16 h. Cooled to RT and diluted with EtOAc (100 ml) and water (60 ml). The mixture was filtered through a pad of celite. The filtrate was washed with brine (60 mL) and then 1:1 brine/water (3×100 mL). The organic layer was dried over magnesium sulfate, filtered, and concentrated to a dark solid. The solid was then triturated with diethyl ether overnight. The solid was collected by filtration, washed with ether and air dried to give N-(2-Chloro-6-cyano-4-methyl-pyridin-3-yl)-propionamide (180 g, 87.5%). MS: 224.1 (APCI)⁺; 222.1 (APCI)⁻

### Step 4. N-(2-Chloro-6-isobutyryl-4-methyl-pyridin-3-yl)-propionamide

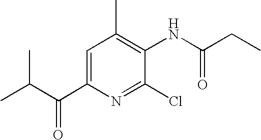

N-(2-Chloro-6-cyano-4-methyl-pyridin-3-yl)-propionamide (50.0 g, 225 mmol) was dissolved in dry THF (500 ml) and the solution was stirred mechanically while being cooled to 0° C. under a nitrogen atm. Isoproplyl magnesium chloride (247 mL of 2.0M solution in THF, 494 mmol) was added slowly, maintaining the temperature below 10° C. After addition of approximately half of the Grignard reagent, copper bromide (0.64 g, 4.5 mmol) was charged to the reaction as a solid. The remaining Grignard reagent was added and the mixture warmed to room temperature with vigorous stirring for 1 hour. Diluted the reaction with THF (200 mL) and the reaction was cooled in an ice bath. The reaction mixture was quenched by slow addition of saturated ammonium chloride solution (1.1 L) and the reaction was stirred until all solids were dissolved. The aqueous layer was separated and washed with EtOAc (2×500 mL). The combined organic layers were dried over magnesium sulfate, filtered, and concentrated in vacuo to an oily residue. The crude oil was dissolved in DCM and loaded onto 200 g silica. Eluted product with DCM (3 L) and concentrated fractions in vacuo to give a solid. The precipitate was triturated with diethyl ether and stirred overnight. A solid was collected by filtration, washed with cold ether, and air dried to give N-(2-Chloro-6-isobutyryl-4-methyl-pyridin-3-yl)-propionamide (29.7 g, 49.1%). MS: 269.1 (APCI)⁺; 267.1 (APCI)⁻

### Step 5. N-(2-Chloro-6-isobutyryl-4-methyl-pyridin-3-yl)-propionimidoyl chloride

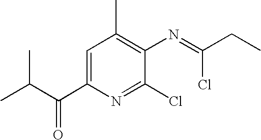

To N-(2-Chloro-6-isobutyryl-4-methyl-pyridin-3-yl)-propionamide (24.02 g, 89.4 mmol) in DCM (75 mL) was added phosphorus pentachloride (19.5 g, 93.8 mmol) slowly. Bubbling occurred. When the bubbling stopped the solution was heated to reflux for 2.5 hours. The mixture was cooled and concentrated in vacuo, and the residue was dried under vacuum overnight to give N-(2-Chloro-6-isobutyryl-4-methyl-pyridin-3-yl)-propionimidoyl chloride in quantitative yield. (25.67 g, 100%). ¹H NMR (400 MHz, CDCl₃) was consistent with product.

### Step 6. (S)—N-(5-Bromo-indan-1-yl)-N′-(2-chloro-6-isobutyryl-4-methyl-pyridin-3-yl)-propionamidine

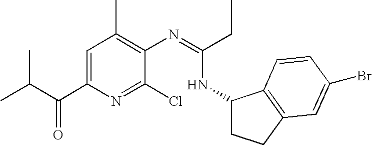

A solution of (S)-5-Bromo-indan-1-ylamine (24.6 g, 116 mmol) and diisopropylethylamine (62.3 mL, 358 mmol) in DCM (150 mL) was cooled to 0° C. in an ice bath. To this solution was added N-(2-Chloro-6-isobutyryl-4-methyl-pyridin-3-yl)-propionimidoyl chloride (25.67 g, 89.38 mmol) in DCM (25 mL) dropwise via an addition funnel. The solution was allowed to warm to 23° C. Monitored by TLC and added (S)-5-Bromo-indan-1-ylamine until the reaction was complete. The mixture was diluted with DCM (100 mL) and run through a short pad of silica gel. Concentrated in vacuo and added a minimal amount of EtOAc and purified on a silica gel column with gradient of EtOAc in heptane 0-40%. Combined and concentrated fractions in vacuo. Remaining EtOAc was azeotroped off with acetonitrile and dried under reduced pressure to give (S)—N-(5-Bromo-indan-1-yl)-N′-(2-chloro-6-isobutyryl-4-methyl-pyridin-3-yl)-propionamidine (35.82 g, 87%). MS: 464.2 (APCI)⁺; 463.2 (APCI)⁻

### Step 7. 1-(2-Ethyl-7-methyl-3-{(S)-5-[2-(1-trityl-1H-tetrazol-5-yl)-phenyl]-indan-1-yl}-3H-imidazo[4,5-b]pyridin-5-yl)-2-methyl-propan-1-one

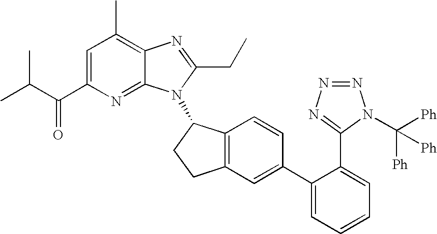

To a solution of (S)—N-(5-bromo-indan-1-yl)-N′-(2-chloro-6-isobutyryl-4-methyl-pyridin-3-yl)-propionamidine (35.82 g, 77.4 mmol) in DME (400 mL) was added 1-trityl-1H-tetrazole-5-boronic acid (33.5 g, 77.4 mmol), triphenylphosphine (8.12 g, 31 mmol), potassium carbonate (32.1 g, 232 mmol), and water (13.9 mL) Nitrogen was bubbled through the mixture for 15 min. Then palladium acetate (1.74 g, 7.74 mmol) was added and the reaction mixture heated at reflux for 4 hours. The reaction was monitored for the Suzuki adduct via LCMS/MS. When the reaction was complete, to this mixture was added 2-dicyclohexylphosphino-2′,6′-dimethoxy-biphenyl (4.77 g, 11.6 mmol), palladium acetate (0.869 g, 3.87 mmol), water (7.0 mL) and potassium carbonate (5.35 g, 38.7 mmol). This mixture was allowed to stir at 80° C. overnight. Additional 2-dicyclohexylphosphino-2′,6′-dimethoxy-biphenyl (3.18 g, 7.74 mmol) and palladium acetate (0.434 g, 1.93 mmol) were added. The reaction was allowed to stir at 80° C. overnight. The reaction was monitored for completion by TLC. The reaction mixture poured onto a pad of silica gel (˜100 g) and washed with ethyl acetate. Concentrated in vacuo and added ˜100 mL ethyl acetate and filtered through Celite®. Celit pad was washed with ethyl acetate and the filtrate concentrated in vacuo. The residue was dissolved in a minimal amount of ethyl acetate, and purified on a silica gel column using a gradient of EtOAc in heptane 10-50% as eluant. Fractions were combined and concentrated to half volume until a solid began to precipitate. The precipitate was collected by filtration, washed with heptane, and air dried to give 1-(2-ethyl-7-methyl-3-{(S)-5-[2-(1-trityl-1H-tetrazol-5-yl)-phenyl]-indan-1-yl}-3H-imidazo[4,5-b]pyridin-5-yl)-2-methyl-propan-1-one (35.65 g, 63%). MS: 492.4 (APCI)⁺; 490.4 (734.5) (APCI)⁻

### Step 8. (S)-1-(2-Ethyl-7-methyl-3-{(S)-5-[2-(1H-tetrazol-5-yl)-phenyl]-indan-1-yl}-3H-imidazo[4,5-b]pyridin-5-yl)-2-methyl-propan-1-ol

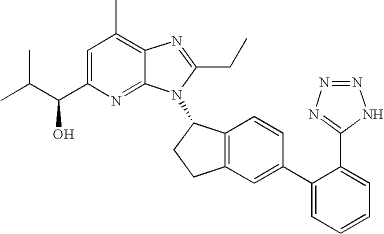

In a glove box, a 100 ml stainless steel vessel was charged with 1-(2-Ethyl-7-methyl-3-{(S)-5-[2-(1-trityl-1H-tetrazol-5-yl)-phenyl]-indan-1-yl}-3H-imidazo[4,5-b]pyridin-5-yl)-2-methyl-propan-1-one (12.00 g, 16.35 mmol), Ru (R-DM-SEGPHOS)(R-DAIPEN) (purchased from Takeda) (0.021 g), KOtBu (0.360 g, 0.032 mmol), 48 ml IPA, and 12 ml THF. The reactor was removed from glove box and placed in reactor stand. The reactor was then pressurized to 50 psi with H₂ and stirred for 24 hours at 23° C. HPLC of an aliquot withdrawn from this reaction showed 94% completion. IPA and THF were then removed in vacuo to give an oily residue. A ˜5 mg sample of this material was removed, diluted with MeOH (5 mL) and refluxed for 2 hours. This subsequent sample was then analyzed by HPLC to determine a diastereomeric excess of 93%. Crude (S)-1-(2-Ethyl-7-methyl-3-{(S)-5-[2-(1H-tetrazol-5-yl)-phenyl]-indan-1-yl}-3H-imidazo[4,5-b]pyridin-5-yl)-2-methyl-propan-1-ol (12.3 g) was then dissolved in 50 mL MeOH and refluxed for 4 hours. Reaction was monitored by HPLC. Once complete, solvent was removed in vacuo and loaded onto 12 g of silica gel. This was then chromatographed with 2.5% MeOH/CH₂Cl₂ to give 6.23 g of desired alcohol (77% yield over two steps) Large scale chromatography can also be effected using 10% IPA/toluene. The solid was crystallized from EtOAc to give white crystals. ¹H NMR (400 MHz, CHLOROFORM-d) δ ppm 0.65 (d, J=7.02 Hz, 3H) 0.95 (d, J=7.02 Hz, 3H) 1.54 (t, J=7.60 Hz, 3H) 1.93-2.04 (m, 1H) 2.38-2.50 (m, 1H) 2.66 (s, 3H) 2.84-2.97 (m, 1H) 3.16 (d, J=7.41 Hz, 2H) 3.22-3.34 (m, 1H) 3.40-3.52 (m, 1H) 4.76 (s, 1H) 4.82 (s, 1H) 6.04 (dd, J=8.97, 4.29 Hz, 1H) 6.57 (d, J=7.41 Hz, 1H) 6.78 (d, J=7.80 Hz, 1H) 6.84 (s, 1H) 7.51-7.58 (m, 2H) 7.59-7.66 (m, 2H) 7.91 (dd, J=8.19. 1.17 Hz, 1H). CHN analysis: C=70.56%; H=6.33%; N=19.86% theoretical, C=70.49%; H=6.34%; N=19.83% Observed.

### Example 10c

### (S)-1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-2-methylpropan-1-ol

### Step 1 Bromo-2-chloro-4-methyl-pyridin-3-ylamine

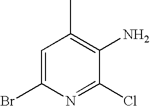

3-Amino-2-chloro-4-methylpyridine (50.0 g, 351 mmol) was dissolved in DCM (500 mL) under nitrogen and cooled the solution in an ice bath to between 0-1° C. Dibromo-5,5-dimethylhydantoin (51.1 g, 179 mmol) was added in 5 portions over 40 min. After the addition was finished, the mixture was stirred at room temperature for 2 hours. The reaction mixture was passed through a short pad of silica gel, eluted with 30% ether/DCM (1 L). The mixture was concentrated and heptane was added. The precipitate was collected by filtration, washed with heptane, and air dried to give 6-Bromo-2-chloro-4-methyl-pyridin-3-ylamine (73.3 g, 94%). MS: 223.0 (APCI)⁺; 220.9 (APCI)⁻

### Step 2 N-(6-Bromo-2-chloro-4-methyl-pyridin-3-yl)-propionamide

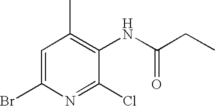

To 6-Bromo-2-chloro-4-methyl-pyridin-3-ylamine (73.3 g, 331 mmol) in toluene (100 mL) was added propionic anhydride (44.5 mL, 347 mmol). The mixture was heated under reflux overnight under nitrogen. The mixture was cooled to 70° C. and then heptane added. Allowed to stir and cool to 23° C. The precipitate was collected by filtration, washed with heptane, and air dried to give N-(6-Bromo-2-chloro-4-methyl-pyridin-3-yl)-propionamide. (86.4 g, 94%). MS: 279.0 (APCI)⁺; 277.0 (APCI)⁻

### Step 3 N-(2-Chloro-6-cyano-4-methyl-pyridin-3-yl)-propionamide

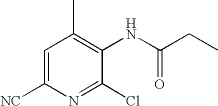

N-(6-Bromo-2-chloro-4-methyl-pyridin-3-yl)-propionamide (255 g, 919 mmol) was dissolved in dry DMF (250 mL) and the solution degassed by bubbling nitrogen through the solution for 10 min. Zinc cyanide (55.0 g, 469 mmol) and Pd(PPh₃)₄ (31.9 g, 27.6 mmol) were added to reaction mixture under a nitrogen blanket. The mixture was heated to 80° C. for 16 h. Cooled to RT and diluted with EtOAc (100 ml) and water (60 ml). The mixture was filtered through a pad of celite. The filtrate was washed with brine (60 mL) and then 1:1 brine/water (3×100 mL). The organic layer was dried over magnesium sulfate, filtered, and concentrated to a dark solid. The solid was then triturated with diethyl ether overnight. The solid was collected by filtration, washed with ether and air dried to give N-(2-Chloro-6-cyano-4-methyl-pyridin-3-yl)-propionamide (180 g, 87.5%). MS: 224.1 (APCI)⁺; 222.1 (APCI)⁻

### Step 4 N-(2-Chloro-6-isobutyryl-4-methyl-pyridin-3-yl)-propionamide

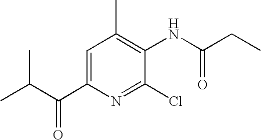

N-(2-Chloro-6-cyano-4-methyl-pyridin-3-yl)-propionamide (50.0 g, 225 mmol) was dissolved in dry THF (500 ml) and the solution was stirred mechanically while being cooled to 0° C. under a nitrogen atm. Isoproplyl magnesium chloride (247 mL of 2.0M solution in THF, 494 mmol) was added slowly, maintaining the temperature below 10° C. After addition of approximately half of the Grignard reagent, copper bromide (0.64 g, 4.5 mmol) was charged to the reaction as a solid. The remaining Grignard reagent was added and the mixture warmed to room temperature with vigorous stirring for 1 hour. Diluted the reaction with THF (200 mL) and the reaction was cooled in an ice bath. The reaction mixture was quenched by slow addition of saturated ammonium chloride solution (1.1 L) and the reaction was stirred until all solids were dissolved. The aqueous layer was separated and washed with EtOAc (2×500 mL). The combined organic layers were dried over magnesium sulfate, filtered, and concentrated in vacuo to an oily residue. The crude oil was dissolved in DCM and loaded onto 200 g silica. Eluted product with DCM (3 L) and concentrated fractions in vacuo to give a solid. The precipitate was triturated with diethyl ether and stirred overnight. A solid was collected by filtration, washed with cold ether, and air dried to give N-(2-Chloro-6-isobutyryl-4-methyl-pyridin-3-yl)-propionamide (29.7 g, 49.1%). MS: 269.1 (APCI)⁺; 267.1 (APCI)⁻

### Step 5 N-(2-Chloro-6-isobutyryl-4-methyl-pyridin-3-yl)-propionimidoyl chloride

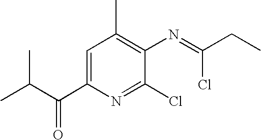

N-(2-Chloro-6-isobutyryl-4-methyl-pyridin-3-yl)-propionamide (200 g, 744 mmol) was dissolved in DCM (1600 mL). The solution was slowly added to a suspension of phosphorus pentachloride (164 g, 781 mmol) in DCM (250 mL). The rate was adjusted to keep the temperature less than 30° C. The resulting solution was stirred at less than 30° C. for 2 hours. The solution was heated under reflux for 12-16 hours. The mixture was concentrated by atmospheric distillation to about 400 mL in volume. Toluene (1000 mL) was added and the solution concentrated by vacuum distillation to about 400 mL in volume. Toluene was again added and the solution concentrated to about 400 mL in volume. The N-(2-Chloro-6-isobutyryl-4-methyl-pyridin-3-yl)-propionimidoyl chloride was used directly as a toluene solution in step 6.

### Step 6 (S)—N-(5-Bromo-indan-1-yl)-N′-(2-chloro-6-isobutyryl-4-methyl-pyridin-3-yl)-propionamidine

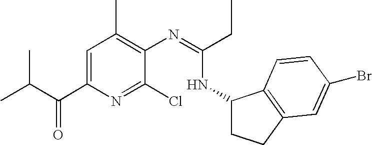

A solution of (S)-5-Bromo-indan-1-ylamine (284 g, 1340 mmol) and triethylamine (188 g, 1860 mmol) in toleune (600 mL) was cooled to 0° C. in an ice bath. To this solution was added the N-(2-Chloro-6-isobutyryl-4-methyl-pyridin-3-yl)-propionimidoyl chloride (744 mmol) solution toluene from step 5. The solution was warmed to 65° C. Monitored by TLC until the reaction was complete. The mixture was cooled and diluted with ethyl acetate (1000 mL) and run through a short pad of silica gel. Concentrated in vacuo and added a minimal amount of EtOAc and purified on a silica gel column with gradient of EtOAc in heptane 0-40%. Combined and concentrated fractions in vacuo. Remaining EtOAc was azeotroped off with acetonitrile and dried under reduced pressure to give (S)—N-(5-Bromo-indan-1-yl)-N′-(2-chloro-6-isobutyryl-4-methyl-pyridin-3-yl)-propionamidine. (317 g, 92%). MS: 464.2 (APCI)⁺; 463.2 (APCI)⁻

### Step 7 1-(2-Ethyl-7-methyl-3-{(S)-5-[2-(1-trityl-1H-tetrazol-5-yl)-phenyl]-indan-1-yl}-3H-imidazo[4,5-b]pyridin-5-yl)-2-methyl-propan-1-one

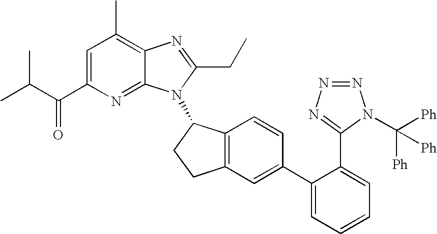

(S)—N-(5-Bromo-indan-1-yl)-N′-(2-chloro-6-isobutyryl-4-methyl-pyridin-3-yl)-propionamidine (300 g, 648 mmol) was dissolved in DME (3000 mL). To this solution was added 1-trityl-1H-tetrazole-5-boronic acid (327 g, 648 mmol), triphenylphosphine (32 g, 130 mmol), potassium carbonate (269 g, 1940 mmol), and water (30 mL) Nitrogen was bubbled through the mixture for 1 hour. Then palladium acetate (14.6 g, 64.8 mmol) was added and nitrogen bubbled through the slurry for 20 minutes. The slurry was heated to 65° C. for 6 hours. The reaction was monitored for the Suzuki adduct via HPLC. When the reaction was complete to this mixture was added 2-(Dicyclohexylphosphino)-2′,4′,6′-tri-1-propyl-1,1′-biphenyl (61.8 g, 130 mmol) and palladium acetate (14.6 g, 64.8 mmol). This mixture was allowed to stir at 65° C. for 12 hours. The reaction was monitored for completion by HPLC. The reaction mixture poured onto a pad of silica gel (˜500 g) and washed with ethyl acetate. Concentrated in vacuo, added minimal amount of ethyl acetate, and purified on a silica gel column with gradient of EtOAc in heptane 10-50%. Eluted with 90% heptane in ethyl acetate to 50% heptane in ethyl acetate. Fractions were combined and concentrated to half volume until a solid began to precipitate. The precipitate was collected by filtration, washed with heptane, and air dried to give 1-(2-Ethyl-7-methyl-3-{(S)-5-[2-(1-trityl-1H-tetrazol-5-yl)-phenyl]-indan-1-yl}-3H-imidazo[4,5-b]pyridin-5-yl)-2-methyl-propan-1-one. (300 g, 63%). MS: 492.4 (APCI)⁺; 490.4 (734.5) (APCI)⁻

### Step 8 (S)-1-(2-Ethyl-7-methyl-3-{(S)-5-[2-(1H-tetrazol-5-yl)-phenyl]-indan-1-yl}-3H-imidazo[4,5-b]pyridin-5-yl)-2-methyl-propan-1-ol

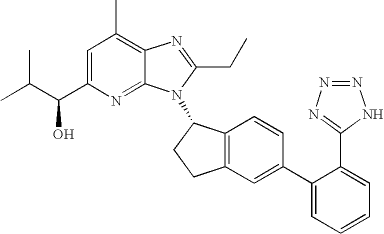

T a 2 L hastelloy pressure vessel was charged with 1-(2-Ethyl-7-methyl-3-{(S)-5-[2-(1-trityl-1H-tetrazol-5-yl)-phenyl]-indan-1-yl}-3H-imidazo[4,5-b]pyridin-5-yl)-2-methyl-propan-1-one (169 g, 230 mmol), KOtBu (5.38 g, 46.1 mmol), isopropanol (800 mL) and THF (200 mL). The solution was purged with nitrogen (50 psi) three times. RuCl₂(S-BINAP)(R-DPEN) (0.58 g)., was weighed out in a nitrogen purged dry box and dissolved in THF (5 mL). The catalyst was injected into the reactor via syringe. The reactor was then pressurized to 50 psi with H₂ and stirred for 24 hours at 23° C. HPLC of an aliquot withdrawn from this reaction showed 98% completion. IPA and THF were then removed in vacuo to give an oily residue. A ˜5 mg sample of this material was removed, diluted with MeOH (5 mL) and refluxed for 2 hours. This subsequent sample was then analyzed by HPLC to determine a diastereomeric excess of 98%. Crude (S)-1-(2-Ethyl-7-methyl-3-{(S)-5-[2-(1H-tetrazol-5-yl)-phenyl]-indan-1-yl}-3H-imidazo[4,5-b]pyridin-5-yl)-2-methyl-propan-1-ol (169 g) was then dissolved in 500 mL MeOH and refluxed for 16 hours. Reaction was monitored by HPLC. Once complete, solvent was removed in vacuo. The residue was then chromatographed with 5% MeOH/CH₂Cl₂ to give 87 g of desired alcohol (77% yield over two steps) Large scale chromatography can also be effected using 10% IPA/toluene. The solid resulting from the chromatography was dissolved in ethyl acetate (500 mL) and the solution concentrated to a foam under vacuum. The residue was then dissolved in ethyl acetate (100 mL) at 65° C. The solution was cooled to 23° C. and stirred for 2 hours. To the resulting thick slurry was added heptane (100 mL) over 30 minutes. The slurry was stirred for 2 hours, filtered and washed with 1:1 ethyl acetate:heptane (100 mL) to give 73.5 g of the desired product.

1H NMR (400 MHz, CHLOROFORM-d) delta ppm 0.65 (d, J=7.02 Hz, 3H) 0.95 (d, J=7.02 Hz, 3H) 1.54 (t, J=7.60 Hz, 3H) 1.93-2.04 (m, 1H) 2.38-2.50 (m, 1H) 2.66 (s, 3H) 2.84-2.97 (m, 1H) 3.16 (d, J=7.41 Hz, 2H) 3.22-3.34 (m, 1H) 3.40-3.52 (m, 1H) 4.76 (s, 1H) 4.82 (s, 1H) 6.04 (dd, J=8.97, 4.29 Hz, 1H) 6.57 (d, J=7.41 Hz, 1H) 6.78 (d, J=7.80 Hz, 1H) 6.84 (s, 1H) 7.51-7.58 (m, 2H) 7.59-7.66 (m, 2H) 7.91 (dd, J=8.19, 1.17 Hz, 1H). CHN analysis. C=70.56%; H=6.33%; N=19.86%

theoretical, C=70.49%; H=6.34%; N=19.83% Observed.

Table 4 lists the 2θ, d-spacings, and relative intensities of all lines in the sample with a relative intensity of >15% for crystalline Form A which can be prepared according to compound preparation methods described above and the polymorph is formed when solvates desolvate on drying, typically when water is present in the crystallizing solvent. Table 5 lists the 2θ, d-spacings, and relative intensities of all lines in the sample with a relative intensity of >15% for crystalline Form B which was prepared from Example 10(c), Step 8 ethyl acetate crystallization above.

TABLE 4
Intensities and Peak Locations of all Diffraction Lines with Relative
Intensities Greater Than 15% for Form A Example 10

| | | Relative |
|---|---|---|
| | | Intensity |
| 2 Theta | d | (>15%) |
| 7.559 | 11.68566 | 18.4 |
| 8.481 | 10.41777 | 32.7 |
| 9.735 | 9.07752 | 22.8 |
| 10.34 | 8.54777 | 27.6 |
| 10.919 | 8.09599 | 44.8 |
| 12.08 | 7.32023 | 24.6 |
| 13.977 | 6.33069 | 100 |
| 14.45 | 6.1247 | 43.1 |
| 16.21 | 5.46346 | 55.2 |
| 16.967 | 5.22127 | 32.5 |
| 17.974 | 4.93113 | 45.2 |
| 18.406 | 4.81627 | 41.3 |
| 19.882 | 4.46198 | 39.1 |
| 20.334 | 4.36375 | 42.8 |
| 21.23 | 4.18165 | 56.5 |
| 21.642 | 4.10288 | 62.9 |
| 22.328 | 3.9784 | 93.6 |
| 24.5 | 3.63035 | 27.8 |
| 25.619 | 3.47432 | 30.4 |
| 26.25 | 3.39216 | 27.9 |
| 26.499 | 3.36091 | 27.4 |
| 27.098 | 3.28792 | 25.3 |
| 28.95 | 3.08164 | 24.1 |
| 29.223 | 3.05351 | 26.2 |
| 30.485 | 2.92989 | 16.8 |
| 31.066 | 2.87641 | 18.4 |
| 31.866 | 2.80599 | 22.2 |

TABLE 5
Intensities and Peak Locations of all Diffraction Lines with Relative
Intensities Greater Than 15% for Form B Example 10

| | | Relative |
|---|---|---|
| | | Intensity |
| 2 Theta | d | (>15%) |
| 10.405 | 8.49509 | 48.1 |
| 11.141 | 7.9356 | 35.3 |
| 11.966 | 7.38989 | 44 |
| 12.765 | 6.92907 | 17.3 |
| 14.163 | 6.24823 | 49.8 |
| 14.742 | 6.00385 | 16.1 |
| 15.933 | 5.55771 | 22.1 |
| 17.707 | 5.00467 | 28.1 |
| 19.614 | 4.52231 | 24.4 |
| 20.228 | 4.38634 | 100 |
| 21.238 | 4.18002 | 40.5 |
| 22.222 | 3.99702 | 22.8 |
| 23.22 | 3.82748 | 23.7 |
| 24.445 | 3.6384 | 29.5 |
| 26.44 | 3.36822 | 21.8 |
| 27.01 | 3.29846 | 23.2 |
| 27.698 | 3.21799 | 16.4 |

### Example 11

### (R)-1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-2-methylpropan-1-ol

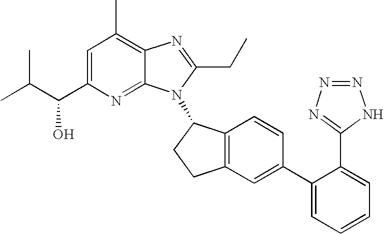

Data for 11 (R)-1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-2-methylpropan-1-ol. ¹H-NMR (400 MHz, CDCl₃) δ ppm 7.95 (d, 1H), 7.65-7.50 (m, 4H), 6.90-6.85 (m, 2H), 6.60 (d, 1H), 6.00 (m, 1H), 4.80 (m, 1H), 3.50 (m, 1H), 3.30 (m, 1H), 3.10 (m, 2H), 2.90 (m, 1H), 2.65 (s, 3H), 2.40 (m, 1H), 1.60 (m, 3H), 1.00 (d, 3H), 0.60 (m, 3H). MS: 494 (M⁺+1). HPLC: 97.31%. Elemental Analysis Cacid for C₂₉H₃₁N₇O.⅔H₂O: C, 68.89; H, 6.45; N, 19.38. Found: C, 68.91; H, 6.43; N, 18.65.

### Example 12

### 3-((1S)-5-(2-(1H-tetrazol-5-yl)phenyl)-2,3-dihydro-1H-inden-1-yl)-2-ethyl-5-((S)-1-methoxy-2-methylpropyl)-7-methyl-3H-imidazo[4,5-b]pyridine

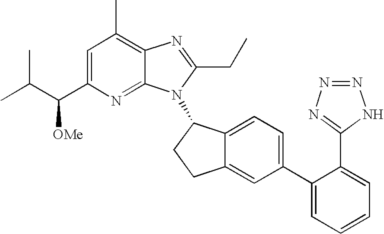

A cooled (0° C.) solution of (S)-1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)-2-methylpropan-1-ol (0.22 g, 0.30 mmol) in THF (5 mL) was treated with sodium hydride (38 mg, 95%) and stirred at 0° C. After 10 min methyl iodide (0.20 mL, 3.00 mmol) was added and the resulting mixture was stirred at RT for 16 h. The reaction was quenched with aqueous ammonium chloride (0.2 mL), diluted with EtOAc (30 mL), dried and concentrated. The residue was purified by silica gel chromatography. The methylated product was refluxed in methanol for 3 h, concentrated. The residue was purified by chromatography to give 3-((1S)-5-(2-(1H-tetrazol-5-yl)phenyl)-2,3-dihydro-1H-inden-1-yl)-2-ethyl-5-((S)-1-methoxy-2-methylpropyl)-7-methyl-3H-imidazo[4,5-b]pyridine (124 mg, 80% over two steps) as a white solid. ¹H-NMR (400 MHz, CDCl₃) δ ppm 8.00 (br s, 1H), 7.70-6.70 (m, 7H), 4.00-2.00 (m, 13H), 1.60-1.00 (m, 10H). MS: 508.29 (M⁺+1). HPLC: 97.81%. Elemental Analysis Cacld for C₃₀H₃₃N₇O.C₆H₁₄: C, 72.82%; H, 7.98; N, 16.09. Found: C, 72.07; H, 7.72; N, 16.51.

### Example 13

### (S)-2-Ethyl-5-(2-methoxy-ethyl)-7-methyl-3-{5-[2-(1H-tetrazol-5-yl)-phenyl]-indan-1-yl}-3H-imidazo[4,5-b]pyridine

### [3-((1S)-5-(2-(1H-tetrazol-5-yl)phenyl)-2,3-dihydro-1H-inden-1-yl)-2-ethyl-5-(2-methoxyethyl)-7-methyl-3H-imidazo[4,5-b]pyridine]

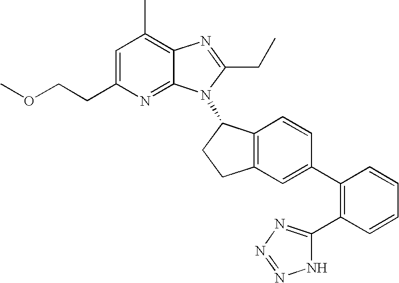

### Step 1. (S)-3-(5-Bromo-indan-1-yl)-2-ethyl-7-methyl-3H-imidazo[4,5-b]pyridine-5-carboxylic acid methyl ester

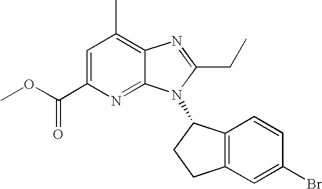

5-Bromo-3-(5-bromo-indan-1-yl)-2-ethyl-7-methyl-3H-imidazo[4,5-b]pyridine (20.12 g, 0.0426 mole); Pd(OAc)₂ (2.06 g, 9.20 mmole, 20 mole %); PPh₃ (2.41 g, 9.20 mmole, 20 mole %); TEA (25.6 mlg, 4 eq) and PdCl₂(PPh₃)₂ (650 mg, 0.92 mmole, 2 mole %) in 500 ml of methanol were charged into a 2000 cc stainless steel reactor. The reactor was purged with nitrogen then with CO, pressurized to 500 psi and set to run at 80° C. for 6 hours. After sampling and analysis, the reactor was re-sealed, purged and pressurized to 500 psi of CO. The reactor was then stirred and heated to 80° C. (3 hours total including heat-up), then again sampled and the reaction was found to be complete. The reactor contents were transferred to a round bottomed flask and solvent was removed in vacuo. 17.40 g of crude desired ester were recovered (91% yield) and used in the next step without further purification. MS: 414.0 (APCI)⁺

### Step 2. (S)-[3-(5-Bromo-indan-1-yl)-2-ethyl-7-methyl-3H-imidazo[4,5-b]pyridin-5-yl]-methanol

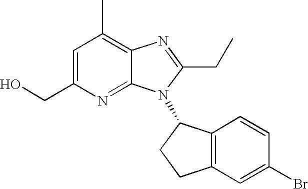

To a cooled (−78° C.) solution of (S)-3-(5-Bromo-indan-1-yl)-2-ethyl-7-methyl-3H-imidazo[4,5-b]pyridine-5-carboxylic acid methyl ester (17.00 g, 41.03 mmol) in THF (500 mL) was added 1 M LiAlH₄ (98.5 mL, 98.5 mmol) over 15 minutes. Stirred at −78° C. for 3 hours then carefully (!) quenched with saturated Na₂SO₄ solution. Aqueous layer extracted with Et₂O (3×75 mL). Organic layers combined and washed with water (30 mL), brine (20 mL) and dried over MgSO₄. Solvent removed in vacuo and residue chromatographed with 50% EtOAc/Hex to give 6.43 g of (S)-[3-(5-Bromo-indan-1-yl)-2-ethyl-7-methyl-3H-imidazo[4,5-b]pyridin-5-yl]-methanol (40.6% yield). 1H NMR (400 MHz, CHLOROFORM-d) δ ppm 1.41 (t, J=7.41 Hz, 3H) 2.58 (s, 1H) 2.69 (s, 3H) 2.74-2.95 (m, 2H) 3.06-3.20 (m, 1H) 3.24-3.40 (m, 2H) 4.72 (s, 2H) 6.31 (s, 1H) 6.76 (d, J=8.19 Hz, 1H) 6.93 (s, 1H) 7.25-7.31 (m, 1H) 7.53 (s, 1H). MS: 386.2 (APCI)⁺

### Step 3. Methanesulfonic acid (S)-3-(5-bromo-indan-1-yl)-2-ethyl-7-methyl-3H-imidazo[4,5-b]pyridin-5-ylmethyl ester

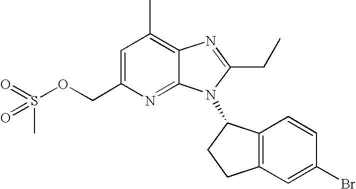

Methanesulfonyl chloride (0.750 mL, 9.65 mmol) was added to a cooled (−78° C.) solution of (S)-[3-(5-Bromo-indan-1-yl)-2-ethyl-7-methyl-3H-imidazo[4,5-b]pyridin-5-yl]-methanol (3.50 g, 8.04 mmol) and diisopropylethylamine (2.50 mL, 14.0 mmol) in CH₂Cl₂ (100 mL) under N₂ atmosphere. Stirred for 2 hours then washed with saturated NaHCO₃ (20 mL) followed by brine (10 mL). Organic layer dried over Na₂SO₄ and filtered. Solvent removed in vacuo to give the mesylate as a yellow foam (4.46 g, 119% yield). Used without further purification. MS: 465.0 (APCI)⁺; 416.0 (APCI)⁺

### Step 4. (S)-[3-(5-Bromo-indan-1-yl)-2-ethyl-7-methyl-3H-imidazo[4,5-b]pyridin-5-yl]-acetonitrile

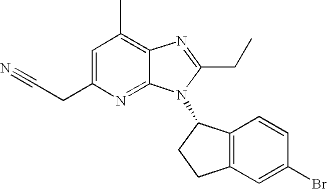

Potassium cyanide (4.46 g, 68.5 mmol) was added to a solution of methanesulfonic acid (S)-3-(5-bromo-indan-1-yl)-2-ethyl-7-methyl-3H-imidazo[4,5-b]pyridin-5-ylmethyl ester (4.41 g, 11.4 mmol) in DMF (25 mL) and heated to 40° C. for 2 hours. Diluted with 500 mL water and extracted with diethyl ether (4×30 mL). Organic layers combined and washed with water (20 mL), brine (20 mL) and dried over MgSO₄. Filtration and removal of solvent in vacuo gave a residue which was chromatographed (25% to 100% EtOAc/Hex) to afford 2.60 g of (S)-[3-(5-Bromo-indan-1-yl)-2-ethyl-7-methyl-3H-imidazo[4,5-b]pyridin-5-yl]-acetonitrile (68% yield for two steps from alcohol)). 1H NMR (400 MHz, CHLOROFORM-d) d ppm 1.37 (t, J=7.42 Hz, 3H) 2.59 (s, 1H) 2.64-2.71 (m, 3H) 2.71-2.85 (m, 2H) 3.03-3.19 (m, 1H) 3.31-3.48 (m, 1H) 3.85-3.89 (m, 2H) 6.32 (s, 1H) 6.74 (d, J=7.81 Hz, 1H) 7.05 (s, 1H) 7.27 (d, J=9.76 Hz, 1H) 7.37 (s, 1H) 7.51 (s, 1H). MS: 396.1 (APCI)⁺, 201.3 (APCI)⁺

### Step 5. (S)-[3-(5-Bromo-indan-1-yl)-2-ethyl-7-methyl-3H-imidazo[4,5-b]pyridin-5-yl]-acetic acid methyl ester

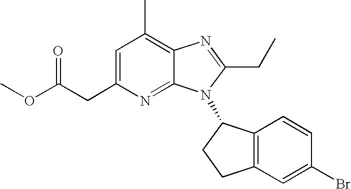

(S)-[3-(5-Bromo-indan-1-yl)-2-ethyl-7-methyl-3H-imidazo[4,5-b]pyridin-5-yl]-acetonitrile (2.60 g, 6.58 mmol) was dissolved in methanol and treated with 4N HCl/Methanol 23° C. for 48 hours. LCMS indicated predominantly desired ester but some nitrile remained. Additional HCl gas was bubbled through the solution. Quenched with aqueous NaHCO₃ (30 mL) and solvent removed in vacuo. Aqueous layer extracted with EtOAc (2×50 mL) and organic layers combined and washed with water (20 mL), brine (20 mL) and dried over MgSO₄. Filtration and removal of solvent under vacuum gave 2.20 g of crude (S)-[3-(5-Bromo-indan-1-yl)-2-ethyl-7-methyl-3H-imidazo[4,5-b]pyridin-5-yl]-acetic acid methyl ester. Used without further purification. 1H NMR (400 MHz, CHLOROFORM-d) δ ppm 1.34 (t, J=7.61 Hz, 3H) 2.56 (d, J=7.81 Hz, 1H) 2.63-2.70 (m, 4H) 2.71-2.86 (m, 2H) 3.01-3.16 (m, 1H) 3.28-3.41 (m, 1H) 3.67 (s, 2H) 3.77-3.83 (m, 2H) 6.35 (s, 1H) 6.75 (d, J=8.20 Hz, 1H) 6.98 (s, 1H) 7.25 (s, 1H) 7.37 (s, 1H) 7.49 (s, 1H). MS: 429.2 (APCI)⁺, 430.1 (APCI)⁺

### Step 6. 2-(S)-[3-(5-Bromo-indan-1-yl)-2-ethyl-7-methyl-3H-imidazo[4,5-b]pyridin-5-yl]-ethanol

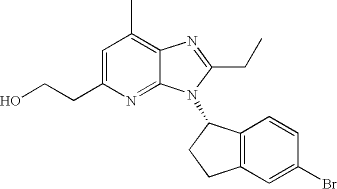

A cooled (−78° C.) solution of (S)-[3-(5-Bromo-indan-1-yl)-2-ethyl-7-methyl-3H-imidazo[4,5-b]pyridin-5-yl]-acetic acid methyl ester (2.20 g, 5.14 mmol) in THF (100 mL) was treated with 1N solution of LAH (6.16 mL, 6.16 mmol) and stirred for 20 min at −78° C. The temperature was then raised to 0° C. (ice bath) for an additional 20 min. The reaction was then carefully quenched with saturated Na₂SO₄ solution and brought to 23° C. Mixture was then further diluted with water (40 mL) and aqueous layer extracted with Et₂O (3×50 mL). Organic layers combined and washed with water (20 mL), brine (20 mL) and dried over MgSO₄. Solvent removed under vacuum. 1H NMR (400 MHz, CHLOROFORM-d) δ ppm 1.41 (t, J=7.42 Hz, 3H) 2.64 (s, 4H) 2.67-2.80 (m, 3H) 2.94 (t, J=4.98 Hz, 2H) 3.04-3.18 (m, 1H) 3.24-3.39 (m, 1H) 3.57 (s, 1H) 3.80 (s, 1H) 3.92 (s, 1H) 6.15 (s, 1H) 6.71 (d, J=8.20 Hz, 1H) 6.83 (s, 1H) 7.25 (s, 1H) 7.53 (s, 1H). MS: 400.1 (APCI)⁺, MS: 206.3 (APCI)⁺

### Step 7. (S)-3-(5-Bromo-indan-1-yl)-2-ethyl-5-(2-methoxy-ethyl)-7-methyl-3H-imidazo[4,5-b]pyridine

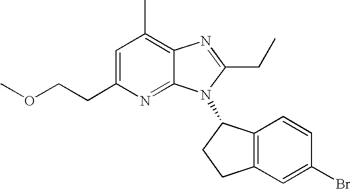

To a solution of 2-(S)-[3-(5-Bromo-indan-1-yl)-2-ethyl-7-methyl-3H-imidazo[4,5-b]pyridin-5-yl]-ethanol (1.98 g, 54.95 mmol) in THF at 0° C. was added sodium hydride (0.396 g 9.89 mmol) and allowed to stir at 0° C. for 15 minutes. Iodomethane (2.75 mL, 29.7 mmol) was then added and the mixture allowed to stir at 23° C. for 16 hours. Solvent removed in vacuo and residue chromatographed to give 1.78 g of (S)-3-(5-Bromo-indan-1-yl)-2-ethyl-5-(2-methoxy-ethyl)-7-methyl-3H-imidazo[4,5-b]pyridine. 1H NMR (400 MHz, CHLOROFORM-d) δ ppm 1.36 (t, J=7.42 Hz, 3H) 2.58 (s, 1H) 2.63 (s, 3H) 2.66 (s, 1H) 2.71-2.84 (m, 2H) 3.00 (t, J=6.64 Hz, 2H) 3.05-3.18 (m, 1H) 3.31 (s, 3H) 3.36 (d, J=9.37 Hz, 1H) 3.54-3.74 (m, 2H) 6.32 (s, 1H) 6.75 (d, J=8.20 Hz, 1H) 6.89 (s, 1H) 7.25 (s, 1H) 7.50 (s, 1H). MS: (APCI)⁺

### Step 8. (S)-2-Ethyl-5-(2-methoxy-ethyl)-7-methyl-3-{5-[2-(1-trityl-1H-tetrazol-5-yl)-phenyl]-indan-1-yl}-3H-imidazo[4,5-b]pyridine

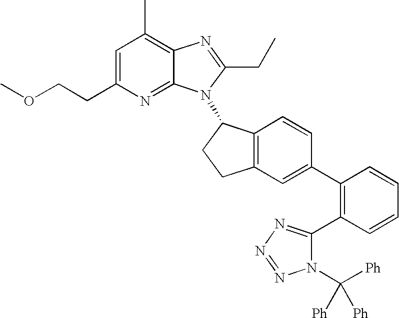

Triphenylphosphine (0.507 g, 1.93 mmol), Pd(OAc)₂ (0.096 g, 0.430 mmol), potassium carbonate (1.93 g, 14.0 mmol), (2-(2-trityl-imidazole)-phenyl boronic acid (0.287 g, 0.663 mmol), (S)-3-(5-Bromo-indan-1-yl)-2-ethyl-5-(2-methoxy-ethyl)-7-methyl-3H-imidazo[4,5-b]pyridine (0.250 g, 0.553 mmol) and water (0.046 mL, 2.54 mmol) were dissolved in DME (10 mL) and degassed for 30 min. The mixture was then heated to 100° C. for 3.5 hours. Solvent removed in vacuo and residue chromatographed to give 2.64 g of (S)-2-Ethyl-5-(2-methoxy-ethyl)-7-methyl-3-{5-[2-(1-trityl-1H-tetrazol-5-yl)-phenyl]-indan-1-yl}-3H-imidazo[4,5-b]pyridine as a off-white foam.

### Step 9. (S)-2-Ethyl-5-(2-methoxy-ethyl)-7-methyl-3-(S)-{5-[2-(1H-tetrazol-5-yl)-phenyl]-indan-1-yl}-3H-imidazo[4,5-b]pyridine

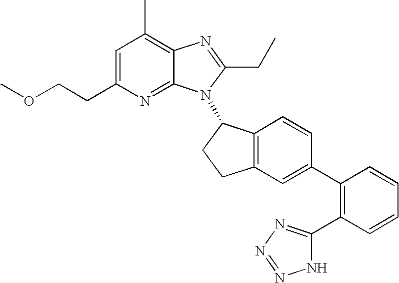

(S)-2-Ethyl-5-(2-methoxy-ethyl)-7-methyl-3-{5-[2-(1-trityl-1H-tetrazol-5-yl)-phenyl]-indan-1-yl}-3H-imidazo[4,5-b]pyridine (2.65 g, 3.49 mmol) was dissolved in MeOH (1 mL) and heated to 80° C. for 16 hours. Solvent removed in vacuo and residue chromatographed (75% EtOAc/heptane with 1% AcOH) to give (S)-2-Ethyl-5-(2-methoxy-ethyl)-7-methyl-3-(S)-{5-[2-(1H-tetrazol-5-yl)-phenyl]-indan-1-yl}-3H-imidazo[4,5-b]pyridine (0.804 g, 47%). 1H NMR (400 MHz, CHLOROFORM-d) δ ppm 1.38 (s, 3H) 2.56 (s, 3H) 2.72 (d, J=5.08 Hz, 2H) 2.92 (s, 1H) 2.98-3.10 (m, 4H) 3.21 (s, 3H) 3.26-3.41 (m, 1H) 3.65 (d, J=6.25 Hz, 1H) 3.76-3.87 (m, 1H) 6.15 (s, 1H) 6.80 (d, J=7.81 Hz, 1H) 6.85-6.95 (m, 2H) 7.15 (s, 1H) 7.44-7.54 (m, 2H) 7.55-7.64 (m, 1H) 7.98 (d, J=6.64 Hz, 1H)

### Example 13

### Alternative Method of Preparation of Example 13

### 2-Ethyl-5-(2-methoxy-ethyl)-7-methyl-3-{(S)-5-[2-(1H-tetrazol-5-yl)-phenyl]-indan-1-yl}-3H-imidazo[4,5-b]pyridine

### [3-((1S)-5-(2-(1H-tetrazol-5-yl)phenyl)-2,3-dihydro-1H-inden-1-yl)-2-ethyl-5-(2-methoxyethyl)-7-methyl-3H-imidazo[4,5-b]pyridine]

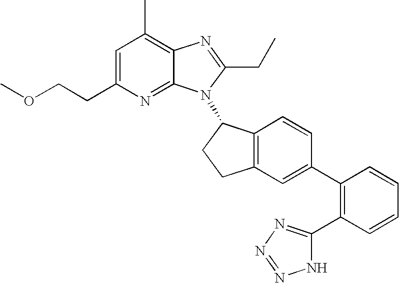

### Step 1. N-(6-Allyl-2-chloro-methyl-pyridin-3-yl)-propionamide

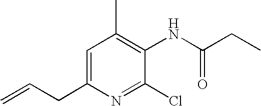

Procedure from Example 8, step 1. 1H NMR (400 MHz, CDCl₃) δ ppm 7.19 (s, 1H), 7.02 (s, 1H), 5.95 (m, 2H), 5.14 (m, 2H), 3.52 (d, 2H), 2.45 (q, 2H), 2.26 (s, 3H), 1.26 (t, 3H). MS: 237, 239 (3:1) (APCI)⁻

### Step 2. N-(6-Allyl-2-chloro-methyl-pyridin-3-yl)-N′—((S)-5-bromo-indan-1-yl)-propionamide

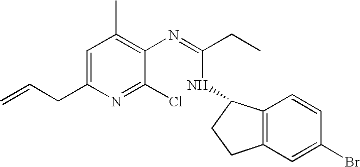

Procedure from Example 8, step 2. MS: 432, 434, 436 (APCI)⁺

### Step 3. 5-Allyl-2-ethyl-7-methyl-3-{(S)-5-[2-(1-trityl-1H-tetrazol-5-yl)-phenyl]-indan-1-yl}-3H-imidazo[4,5-b]pyridine

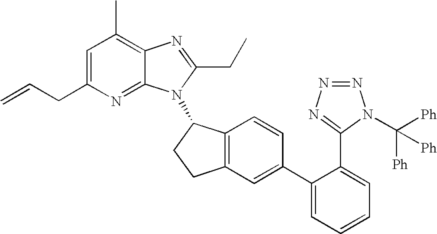

Procedure from Example 8, step 3. MS: 704.4 (APCI)⁺

### Steps 4 and 5. 2-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)ethanol

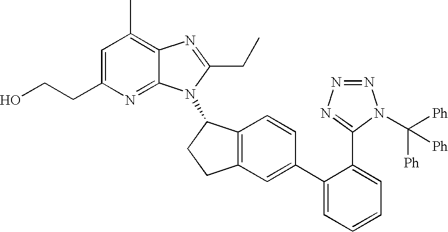

5-Allyl-2-ethyl-7-methyl-3-{(S)-5-[2-(1-trityl-1H-tetrazol-5-yl)-phenyl]-indan-1-yl}-3H-imidazo[4,5-b]pyridine (2.2 g, 2.84 mmol) was dissolved in DCM (25 mL) and methanol (25 mL), the solution was cooled to −50° C. Ozone (O3/O2) generated using (Deizone-LG-7, with full power, flow rate 1 L/min) was bubbled through the reaction mixture for 20 min and resulted in the solution color changing to purple. The reaction was warmed to RT, solvent was removed under vacuo to give a crude ozonide adduct (2.3 g) as a light color foam. MS: 754.3 (APCI)⁺

The crude ozonide adduct (15.81 g, 21.03 mmol) was dissolved in 50 mL of THF and treated with LAH (1M in THF, 21 mL, 21 mmol) at 0° C. in an ice-bath. The mixture was stirred at RT for 2 h. The reaction was quenched with saturated NaHCO₃ (25 mL) and stirred for 15 mins. The layers were separated and the organic layer washed with water, brine, dried with MgSO₄, filtered, and the solvent evaporated to give an oil that hardened to a foam solid of 2-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)ethanol (12.77 g, 85.80% yield). MS: 708.3 (APCI)⁺

### Step 6. 2-ethyl-5-(2-methoxyethyl)-7-methyl-3-((1S)-5-(2-(1-trityl-1H-tetrazol-5-yl)phenyl)-2,3-dihydro-1H-inden-1-yl)-3H-imidazo[4,5-b]pyridine

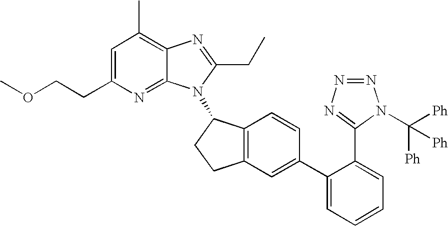

2-(2-ethyl-7-methyl-3-{(S)-5-[2-(1-trityl-1H-tetrazol-5-yl)-phenyl]-indan-1-yl}-3H-imidazo[4,5-b]pyridin-5-yl)-ethanol (12.75 g, 18.01 mmol) was dissolved in 50 mL THF and treated with NaH (2.16 g, 54 mmol). The mixture was stirred at RT for 2 hr and then slowly added with MeI (5.62 mL, 90.1 mmol) and left to stir at RT overnight. The reaction was quenched with saturated NaHCO₃ (25 mL) and stirred for 15 mins. The layers were separated and the organic layer washed with water, brine, dried with MgSO₄, filtered, and the solvent evaporated to give an oil. The crude oil was purified by MPLC on a silica gel column using heptane and ethyl acetate. The pure fractions were collected and the solvent evaporated to give a yellow foam solid of 2-ethyl-5-(2-methoxy-ethyl)-7-methyl-3-{(S)-5-[2-(1-trityl-1H-tetrazol-5-yl)-phenyl]-indan-1-yl}-3H-imidazo[4,5-b]pyridine (3.42 g, 26.3%). MS: 722.3 (APCI)⁺

### Step 7. 2-Ethyl-5-(2-methoxy-ethyl)-7-methyl-3-{(S)-5-[2-(1H-tetrazol-5-yl)-phenyl]-indan-1-yl}-3H-imidazo[4,5-b]pyridine

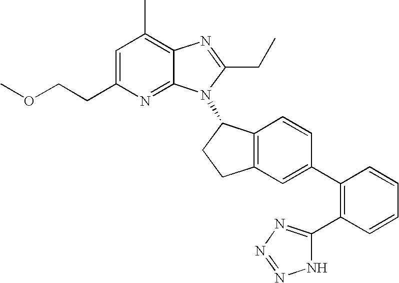

A solution of 2-ethyl-5-(2-methoxy-ethyl)-7-methyl-3-{(S)-5-[2-(1-trityl-1H-tetrazol-5-yl)-phenyl]-indan-1-yl}-3H-imidazo[4,5-b]pyridine (4.20 g, 5.82 mmol) in 75 mL MeOH was refluxed under nitrogen for 2 hrs. The solvent was removed under vacuo. The crude oil was purified by MPLC on a silica gel column using a step gradient of methanol in dichloromethane. The pure fractions were collected and the solvent evaporated to give a white foam solid of 2-ethyl-5-(2-methoxy-ethyl)-7-methyl-3-{(S)-5-[2-(1H-tetrazol-5-yl)-phenyl]-indan-1-yl}-3H-imidazo[4,5-b]pyridine (2.28 g, 81.7%). ¹HNMR (400 MHz, CDCl₃): δ ppm 8.00 (d, 1H), 7.70-7.40 (m, 3H), 7.10 (s, 1H), 7.00-6.70 (m, 3H), 3.90 (br s, 1H), 3.65 (br s, 1H), 3.40-2.40 (m, 14H), 1.40 (m, 3H). HPLC: 98.97%. MS: 480.03 (M⁺+1). Elemental Analysis Cacld for C₂₈H₂₉N₇O.0.5 hexane: C, 71.24; H, 6.94; N, 18.76. Found: C, 70.80; H, 7.08; N, 17.91. MS: 480.2 (APCI)⁺, 478.3 (APCI)⁻. HPLC showed >98% purity. Retention time=10.46 minutes; method 90 to 10% 20 minutes 254 nM (detection wavelength).

Table 6 lists the 2θ, d-spacings, and relative intensities of all lines in the sample with a relative intensity of >15% for crystalline Form A Example 13.

TABLE 6
Intensities and Peak Locations of all Diffraction Lines with Relative
Intensities Greater Than 15% for Form A Example 13

| | | Relative |
|---|---|---|
| | | Intensity |
| 2 Theta | d | (>15%) |
| 9.414 | 9.38633 | 100 |
| 11.591 | 7.62827 | 43.2 |
| 12.683 | 6.97358 | 19.3 |
| 13.515 | 6.54641 | 18.4 |
| 14.216 | 6.22492 | 40 |
| 15.363 | 5.76269 | 35.7 |
| 16.07 | 5.51072 | 45.1 |
| 17.836 | 4.9689 | 22.7 |
| 18.31 | 4.84122 | 15.6 |
| 19.117 | 4.63877 | 29.6 |
| 19.869 | 4.46488 | 30.7 |
| 20.986 | 4.2296 | 62.3 |
| 21.682 | 4.09535 | 38.2 |
| 23.183 | 3.83355 | 54.6 |
| 24.081 | 3.69254 | 46.2 |
| 24.669 | 3.6059 | 45.8 |
| 25.618 | 3.47438 | 39.4 |
| 26.287 | 3.38749 | 23.2 |
| 27.603 | 3.22891 | 16.2 |
| 28.694 | 3.10858 | 23.4 |

### Example 13a

### 2-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)ethanol

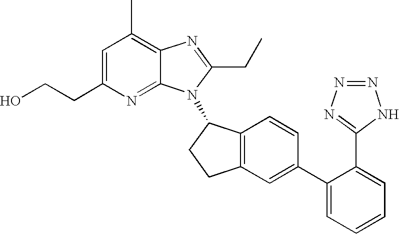

A solution of the 2-(2-ethyl-7-methyl-3-{(S)-5-[2-(1-trityl-1H-tetrazol-5-yl)-phenyl]-indan-1-yl}-3H-imidazo[4,5-b]pyridin-5-yl)-ethanol (4 g, 5.7 mmol) in methanol was refluxed for 24 h. The solvent was removed and the residue purified by silica gel chromatography to give product (1.1 g) in 42% yield. 1H NMR (400 MHz, CHLOROFORM-d) δ ppm 1.55 (t, J=7.60 Hz, 3H) 2.70 (br. s., 3H) 2.73 (s, 2H) 2.91-3.02 (m, 1H) 3.02-3.15 (m, 3H) 3.16-3.34 (m, 3H) 3.75-3.88 (m, 1H) 4.03 (t, J=8.38 Hz, 1H) 5.02 (br. s., 1H) 6.01 (t, J=8.58 Hz, 1H) 6.87 (br. s., 1H) 6.90 (d, J=7.80 Hz, 1H) 6.99 (d, J=7.02 Hz, 1H) 7.28 (s, 1H) 7.44-7.50 (m, 1H) 7.51-7.64 (m, 2H) 8.13 (d, J=8.97 Hz, 1H).

### Example 14

### 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)propan-2-one

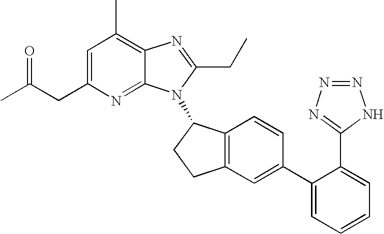

### Step 1. 1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)propan-2-one

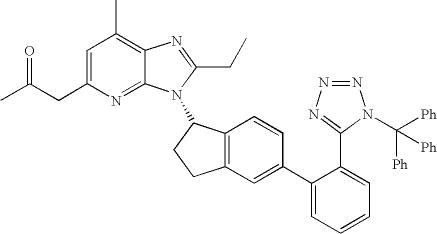

A solution of (S)-5-bromo-2-ethyl-7-methyl-3-{5-[2-(1-trityl-1H-tetrazol-5-yl)-phenyl]-indan-1-yl}-3H-imidazo[4,5-b]pyridine (1.34 mmol) in 25 mL in anhydrous toluene was treated with the isopropenyl acetate (1.61 mmol) followed by the tributyltin methoxide (1.61 mmol), (2′-diphenylphosphanyl-biphenyl-2-yl)-dimethyl-amine ligand (0.05 mmol) and bis(dibenzylideneacetone)palladium(II) catalyst (0.01 mmol). The system was purged with N₂ for 3 min and heated at 90° C. The reaction had an initial dark red color and upon heating turned a light yellow green color. The reaction mixture was heated for six hours. TLC in 40% EtOAc indicated the consumption of the starting material. The reaction mixture was cooled to room temperature and concentrated. Flash chromatography in 50% EtOAc/Heptane yielded 1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)propan-2-one as a yellow foam (0.750 g, 77.4% yield). APCI MS: 720 (M+H)

Step 2. A solution of 1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)propan-2-one in methanol was refluxed in an analogous manner as before to remove the trityl protecting group and give 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)propan-2-one.

### Example 15

### 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)propan-2-ol

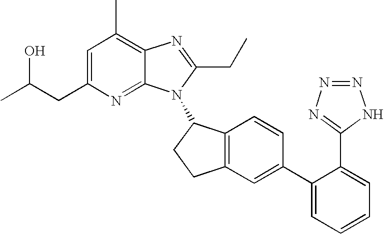

### Step 1. 1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)propan-2-ol

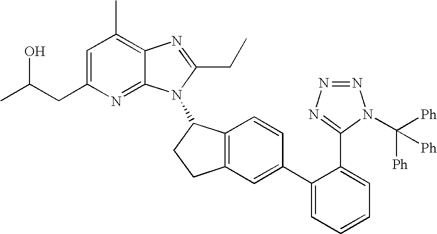

A solution of 1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)propan-2-one (0.412 mmol) in isopropanol (15 mL) at 0° C. was treated with sodium borohydride (0.838 mmol) and the mixture was stirred for 3 hours as the ice bath warmed to room temperature. TLC in 3% MeOH/DCM indicated the consumption of the starting material. 5 mL of water was added and the system was stirred for 5 min. The solution was concentrated and taken back up in 50 mL of DCM and transferred to a separatory funnel. The organic layer was washed with saturated ammonium chloride, water and dried organic phase over sodium sulfate. Flash chromatography in 70% EtOAc/Heptane yielded 0.289 g of desired product as a white foam. APCI Mass Spec.: 722 (M+H).

### Step 2. 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)propan-2-ol

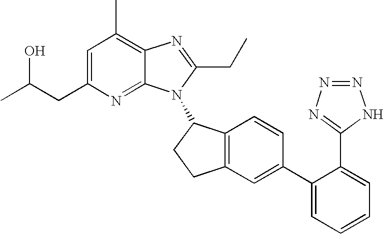

A solution of 1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)propan-2-ol (0.410 mmol) in MeOH (10 mL) was refluxed overnight. The reaction cooled to room temperature and was concentrated. Flash chromatography in 5% MeOH/DCM yielded a light yellow solid. The solid was taken up in ether and heptane was added dropwise until 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)propan-2-ol precipitated as a white solid (0.136 g). 1H NMR (400 MHz, CHLOROFORM-d) δ ppm 1.13 (d, J=6.44 Hz) 1.17 (d, J=6.25 Hz) 1.52 (t, J=7.52 Hz) 2.65 (d, J=3.90 Hz) 2.68 (s) 2.74-2.81 (m) 2.87-2.96 (m) 3.00-3.17 (m) 3.18-3.32 (m) 3.93-4.02 (m) 4.28 (t, J=6.44 Hz) 5.61 (d, J=9.57 Hz) 5.90-6.03 (m) 6.77-6.82 (m) 6.84 (d, J=7.81 Hz) 6.88-6.96 (m) 7.01 (d, J=8.20 Hz) 7.22 (s) 7.46-7.64 (m) 8.11 (t, J=7.61 Hz). APCI Mass Spec.: 494 (M+H).

### Example 15

### Alternative Method. 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)propan-2-ol

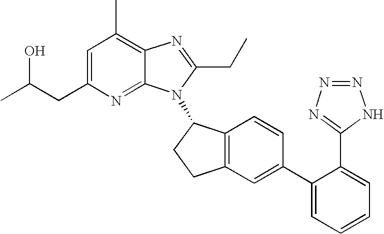

### Step 1 and 2. 1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)propan-2-ol

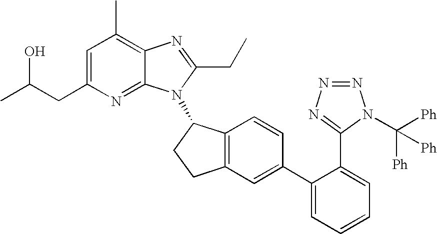

A solution of 5-allyl-2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridine) (Example 9, Step 3, 2.2 g, 2.84 mmol) in dichloromethane (25 mL) and methanol (25 mL) was cooled to −50° C. Ozone (O3/O2) was generated via (Delzone-LG-7, with full power, flow rate 1 L/min) and bubbled into the reaction solution for 20 min; the solution color changed to purple. The reaction was warmed to RT, solvent was removed under vacuo to give a crude the ozonide adduct (2.3 g) as a light color foam. MS: 754.3 (APCI)⁺

A solution of the resulting crude ozonide adduct (0.2 g, 0.27 mmol) in THF (5 mL) at RT was treated with slow addition of methylmagesium chloride (3M in THF, 0.5 mL, 1.33 mmol). The mixture was stirred at RT for 2 h. The reaction was quenched with concentrated sodium bicarbonate (2 mL) and the THF layer separated. The crude product was purified by MPLC on a silica gel column using a step gradient of ethyl acetate in hexanes of 15-75%. Pure fractions were combined and solvent evaporated to give a gummy 1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)propan-2-ol (140 mg, 73% yield). MS: 722.4 (APCI)⁺

### Step 3. 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)propan-2-ol

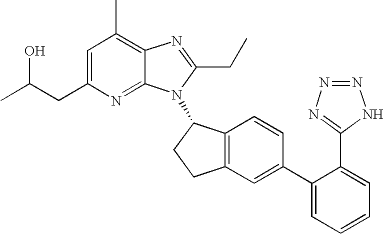

A solution of 1-(2-ethyl-7-methyl-3-{(S)-5-[2-(1-trityl-1H-tetrazol-5-yl)-phenyl]-indan-1-yl}-3H-imidazo[4,5-b]pyridin-5-yl)-propan-2-ol (0.14 g, 0.19 mmol) in methanol (15 mL) was refluxed under nitrogen for 3 hours. The solvent was removed under vacuo. Ethyl acetate (5 mL) was added to the residue, and stirred at RT for 30 min. The precipitate was filtered, to give a white solid 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)propan-2-ol (40 mg, 43%). 1H NMR (400 MHz, CHLOROFORM-d) δ ppm 1.13 (d, J=6.44 Hz) 1.17 (d, J=6.25 Hz) 1.52 (t, J=7.52 Hz) 2.65 (d, J=3.90 Hz) 2.68 (s) 2.74-2.81 (m) 2.87-2.96 (m) 3.00-3.17 (m) 3.18-3.32 (m) 3.93-4.02 (m) 4.28 (t, J=6.44 Hz) 5.61 (d, J=9.57 Hz) 5.90-6.03 (m) 6.77-6.82 (m) 6.84 (d, J=7.81 Hz) 6.88-6.96 (m) 7.01 (d, J=8.20 Hz) 7.22 (s) 7.46-7.64 (m) 8.11 (t, J=7.61 Hz). MS: 480.2 (APCI)⁺; 478.2 (APCI)⁻ HPLC showed >96% purity. Retention time=10.23 minutes; method 90 to 10% 20 minutes 254 nM (detection wavelength).

### Example 15a

### (S)-1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)propan-2-ol (preferred compound)

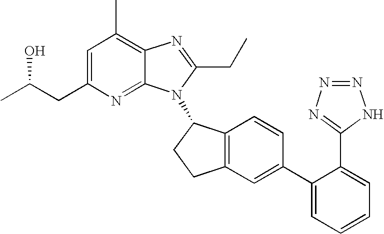

### Example 15b

### (R)-1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)propan-2-ol (preferred compound)

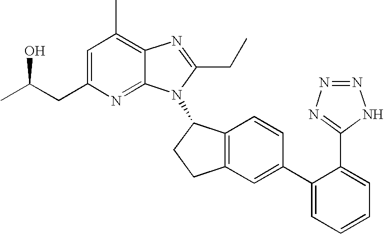

The diastereomeric mixture of 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)propan-2-ol were obtained by separation of the diastereomer mixture via supercritical fluid chromatography to give the pure diastereomers 15a and 15b. The absolute stereochemistry of Example 15a was determined by small molecule x-ray crystallography. APCI Mass Spec.: 494 (M+H).

### Example 16

### 1-((S)-2-Ethyl-7-methyl-3-{5-[2-(1H-tetrazol-5-yl)-phenyl]-indan-1-yl}-3H-imidazo[4,5-]pyridin-5-yl)-2-methyl-propan-2-ol

### [1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-2-methylpropan-2-ol]

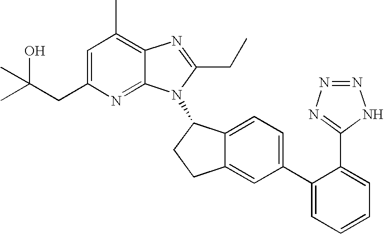

### Step 1. 1-((S)-2-Ethyl-7-methyl-3-{5-[2-(1-trityl-1H-tetrazol-5-yl)-phenyl]-indan-1-yl}-3H-imidazo[4,5-b]pyridin-5-yl)-propan-2-one

### [1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)propan-2-one]

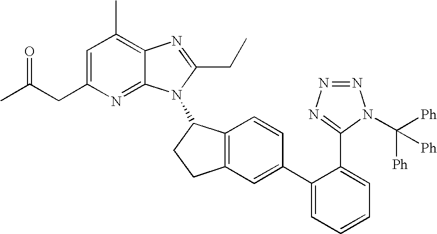

A solution of (S)-5-bromo-2-ethyl-7-methyl-3-(5-[2-(1-trityl-1H-tetrazol-5-yl)-phenyl]-indan-1-yl)-3H-imidazo[4,5-b]pyridine (1.34 mmol) in 25 mL in anhydrous toluene was treated with the isopropenyl acetate (1.61 mmol) followed by the tributyltin methoxide (1.61 mmol), (2′-diphenylphosphanyl-biphenyl-2-yl)-dimethyl-amine ligand (0.05 mmol) and bis(dibenzylideneacetone)palladium(II) catalyst (0.01 mmol). The system was purged with N₂ for 3 min and heated at 90° C. The reaction had an initial dark red color and upon heating turned a light yellow green color. The reaction mixture was heated for six hours. TLC in 40% EtOAc indicated the consumption of the starting material. The reaction mixture was cooled to room temp and concentrated. Flash chromatography in 50% EtOAc/Heptane yielded 1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)propan-2-one as a yellow foam (0.750 g, 77.4% yield). APCI MS: 720 (M+H)

### Step 2. 1-((S)-2-Ethyl-7-methyl-3-{5-[2-(1-trityl-1H-tetrazol-5-yl)-phenyl]-indan-1-yl}-3H-imidazo[4,5-b]pyridin-5-yl)-2-methyl-propan-2-ol

### [1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)-2-methylpropan-2-ol]

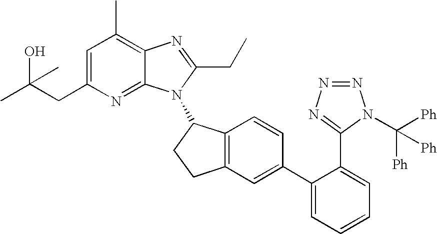

To a cold (0° C.) solution of 1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)propan-2-one (0.417 mmol) in anhydrous THF (10 mL) was added methylmagnesium bromide (0.458 mmol, 1.4 M in THF). The reaction mixture was warmed to room temperature and stirred for 2 hours. TLC and APCI Mass Spec showed partial consumption of starting material. Additional methylmagnesium bromide (0.458 mmol) was added at 0° C., and then the mixture was allowed to warm to room temp and stirred for 2 hours. TLC in 40% EtOAc/HEPTANE showed mostly desired product. The reaction mixture was quenched with 20 mL of saturated ammonium chloride. The reaction mixture was transferred to a separatory funnel and diluted with ethyl acetate (20 mL). Washed with sat ammonium chloride (1×), water (2×) and brine (1×). Dried over sodium sulfate and concentrated to a light yellow foam. TLC showed desired product with almost the same Rf as the starting ketone. A flash column in 45% EtOAC/Heptane provided 104 mg of 1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)-2-methylpropan-2-ol. APCI Mass Spec.: 736 (M+H).

### Step 3. 1-((S)-2-Ethyl-7-methyl-3-{5-[2-(1H-tetrazol-5-yl)-phenyl]-indan-1-yl}-3H-imidazo[4,5-]pyridin-5-yl)-2-methyl-propan-2-ol

### [1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-2-methylpropan-2-ol]

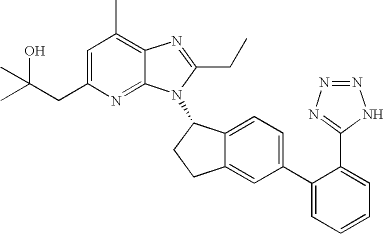

A solution of 1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)-2-methylpropan-2-ol (0.130 mmol) in MeOH (10 mL) was refluxed overnight. The reaction was cooled to room temperature and concentrated. Flash chromatography in 5% MeOH/DCM yielded 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-2-methylpropan-2-ol (0.052 g, 81% yield) as a white foam. APCI Mass Spec.: 494 (M+H). 1H NMR (400 MHz, CHLOROFORM-d) δ ppm 0.92 (s, 3H) 1.25 (s, 3H) 1.53 (t, J=7.61 Hz, 3H) 2.62 (s, 1H) 2.66 (s, 3H) 2.93 (s, 2H) 2.96-3.05 (m, 1H) 3.13 (q, J=7.61 Hz, 3H) 5.31 (s, 2H) 5.82 (s, 1H) 5.97 (t, J=8.79 Hz, 1H) 6.77 (s, 1H) 6.92 (d, J=7.81 Hz, 1H) 7.07-7.13 (m, 2H) 7.46-7.57 (m, 2H) 7.56-7.63 (m, 1H).

### Example 16

### 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-2-methylpropan-2-ol. Alternative method 2 from (S)-3-(5-bromo-2,3-dihydro-1H-inden-1-yl)-5-chloro-2-ethyl-7-methyl-3H-imidazo[4,5-b]pyridine

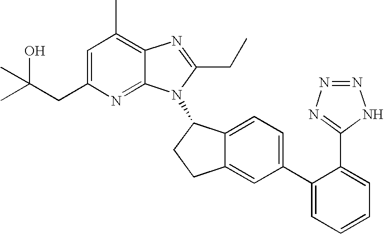

### Step 1. 5-chloro-2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridine

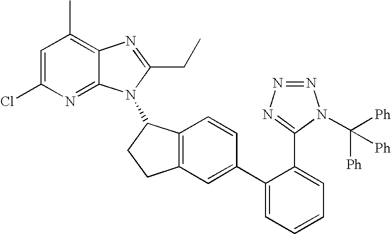

A solution of (S)-3-(5-bromo-2,3-dihydro-1H-inden-1-yl)-5-chloro-2-ethyl-7-methyl-3H-imidazo[4,5-b]pyridine (10.6 g, 27.1 mmol) in anhydrous DME (100 mL) and water (10 mL) under a N2 atmosphere was treated with the tetrazolylphenyl borate (14.1 g, 32.6 mmol), potassium carbonate (11.2 g, 81.4 mmol) and triphenylphosphine (2.85 g, 10.9 mmol). The mixture was deoxygenate by bubbling N2 (g) through the mixture for 10 min. Palladium (II) acetate (0.61 g, 2.71 mmol) was added and the reaction heated at reflux overnight. The reaction mixture was cooled and partitioned between EtOAc and water, washed with saturated aqueous sodium chloride, and the organic layer dried over Mg SO4. The organic residue was purified via silica gel chromatography using 40-90% EtOAc in heptane as eluant to give 5-chloro-2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridine (16.6 g, 88% yield).

### Step 2. 1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)propan-2-one

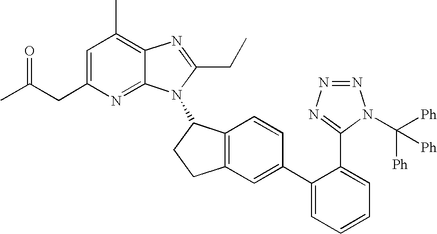

A solution of 5-chloro-2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridine (16.6 g, 23.8 mmol) in anhydrous toluene (100 mL) was deoxygenated with bubbling N2 (g). Isopropenyl acetate (3.92 mL, 35.6 mmol) was added to the reaction mixture followed by addition of dicyclohexylphosphino-2′,6′-dimethoxy-1,1′biphenyl (975 mg, 2.38 mmol) and then tri-n-butyl tin methoxide (10.3 mL, 35.6 mmol) and bis(dibenzylidieneacetone)palladium (0) (218 mg, 0.24 mmol). The dark red solution was heated to 100 C and stirred overnight. The solution was concentrated in vacuo and the residue purified via silica gel chromatography using 50-100% EtOAc in heptanes to give 1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)propan-2-one (13.75 g, 80% yield). APCI MS: 720 (M+H)

Steps 3 and 4. 1-((S)-2-Ethyl-7-methyl-3-{5-[2-(1H-tetrazol-5-yl)-phenyl]-indan-1-yl}-3H-imidazo[4,5-]pyridin-5-yl)-2-methyl-propan-2-ol was completed via an identical procedure as in Example 16, Method 1. 1H NMR (400 MHz, CHLOROFORM-d) δ ppm 0.92 (s, 3H) 1.25 (s, 3H) 1.53 (t, J=7.61 Hz, 3H) 2.62 (s, 1H) 2.66 (s, 3H) 2.93 (s, 2H) 2.96-3.05 (m, 1H) 3.13 (q, J=7.61 Hz, 3H) 5.31 (s, 2H) 5.82 (s, 1H) 5.97 (t, J=8.79 Hz, 1H) 6.77 (s, 1H) 6.92 (d, J=7.81 Hz, 1H) 7.07-7.13 (m, 2H) 7.46-7.57 (m, 2H) 7.56-7.63 (m, 1H). APCI Mass Spec.: 494 (M+H).

Table 7 lists the 2θ, d-spacings, and relative intensities of all lines in the sample with a relative intensity of >15% for crystalline Form A Example 16. Table 8 lists the 2θ, d-spacings, and relative intensities of all lines in the sample with a relative intensity of >15% for crystalline Form B Example 16.

TABLE 7
Intensities and Peak Locations of all Diffraction Lines with Relative
Intensities Greater Than 15% for Form A Example 16

| | | Relative |
|---|---|---|
| | | Intensity |
| 2 Theta | d | (>15%) |
| 7.607 | 11.61171 | 39.1 |
| 10.126 | 8.72859 | 36.4 |
| 11.103 | 7.96246 | 45.7 |
| 11.879 | 7.44389 | 29.3 |
| 12.522 | 7.06303 | 84.7 |
| 14.081 | 6.28448 | 97.5 |
| 15.8 | 5.6043 | 24 |
| 16.172 | 5.47622 | 40.5 |
| 17.174 | 5.15893 | 26.1 |
| 18.48 | 4.79705 | 90.6 |
| 19.462 | 4.5573 | 63.8 |
| 20.123 | 4.40895 | 66.3 |
| 20.457 | 4.33788 | 78.5 |
| 21.571 | 4.11628 | 100 |
| 22.904 | 3.87966 | 59.1 |
| 23.55 | 3.77461 | 39.8 |
| 24.846 | 3.58061 | 26.6 |
| 25.492 | 3.49122 | 28.1 |
| 26.093 | 3.41221 | 23.2 |
| 26.785 | 3.32565 | 29.4 |
| 27.454 | 3.24605 | 35.8 |
| 28.444 | 3.13527 | 49.9 |
| 30.16 | 2.96072 | 21.1 |
| 30.72 | 2.90798 | 19.1 |
| 30.979 | 2.88425 | 19.9 |
| 31.737 | 2.81708 | 21.6 |
| 31.907 | 2.80251 | 23.2 |
| 33.049 | 2.70823 | 15.6 |
| 34 | 2.63459 | 18.1 |
| 34.3 | 2.61222 | 19.3 |

TABLE 8
Intensities and Peak Locations of all Diffraction Lines with Relative
Intensities Greater Than 15% for Form B Example 16

| | | Relative |
|---|---|---|
| | | Intensity |
| 2 Theta | d | (>15%) |
| 7.35 | 12.01698 | 21.2 |
| 7.768 | 11.37221 | 23 |
| 10.407 | 8.49308 | 24.4 |
| 11.587 | 7.63078 | 58.7 |
| 12.491 | 7.0807 | 69.8 |
| 13.484 | 6.56114 | 33.9 |
| 14.477 | 6.11337 | 45.5 |
| 15.883 | 5.57535 | 26.1 |
| 16.505 | 5.3665 | 30.5 |
| 17.037 | 5.20014 | 28.9 |
| 18.831 | 4.70844 | 35.5 |
| 19.718 | 4.49859 | 100 |
| 20.719 | 4.28351 | 67.3 |
| 21.821 | 4.06963 | 53.1 |
| 22.929 | 3.87537 | 49.6 |
| 23.617 | 3.76403 | 41.8 |
| 24.698 | 3.60165 | 27.1 |
| 25.792 | 3.45137 | 34.4 |
| 26.809 | 3.32263 | 31.4 |
| 27.557 | 3.23412 | 29.9 |
| 28.407 | 3.13925 | 25.6 |

### Example 17

### 3-((1S)-5-(2-(1H-tetrazol-5-yl)phenyl)-2,3-dihydro-1H-inden-1-yl)-2-ethyl-5-(2-methoxypropan-2-yl)-7-methyl-3H-imidazo[4,5-b]pyridine

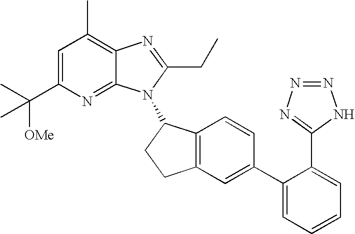

### Step 1 and 2. (S)-3-(5-bromo-2,3-dihydro-1H-inden-1-yl)-2-ethyl-5-(2-methoxypropan-2-yl)-7-methyl-3H-imidazo[4,5-b]pyridine

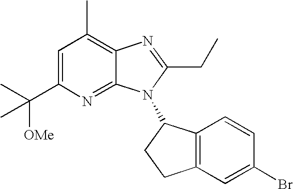

(S)-methyl 3-(5-bromo-2,3-dihydro-1H-inden-1-yl)-2-ethyl-7-methyl-3H-imidazo[4,5-b]pyridine-5-carboxylate was treated with methyl magnesium bromide to give (S)-2-(3-(5-bromo-2,3-dihydro-1H-inden-1-yl)-2-ethyl-7-methyl-3H-imidazo[4,5-b]pyridin-5-yl)propan-2-ol. A solution of (S)-2-(3-(5-bromo-2,3-dihydro-1H-inden-1-yl)-2-ethyl-7-methyl-3H-imidazo[4,5-b]pyridin-5-yl)propan-2-ol (105 mg, 0.25 mmol) in THF (5 mL) was added NaH (13 mg, 0.50 mmol) and MeI (50 μL, 0.75 mmol). The mixture was stirred at RT for 48 h and concentrated. The residue was purified by silica gel chromatography to give (S)-3-(5-bromo-2,3-dihydro-1H-inden-1-yl)-2-ethyl-5-(2-methoxypropan-2-yl)-7-methyl-3H-imidazo[4,5-b]pyridine (110 mg, 100%). ¹H NMR (CDCl₃, 400 MHz) δ ppm δ 7.50 (s, 1H), 7.22 (d, 1H), 7.20 (s, 1H), 6.73 (d, 1H), 6.20 (br s, 1H), 3.40 (m, 1H), 3.10 (m, 4H), 3.00-2.60 (m, 7H), 1.60-1.20 (m, 9H).

### Steps 3 & 4. 3-((1S)-5-(2-(1H-tetrazol-5-yl)phenyl)-2,3-dihydro-1H-inden-1-yl)-2-ethyl-5-(2-methoxypropan-2-yl)-7-methyl-3H-imidazo[4,5-b]pyridine

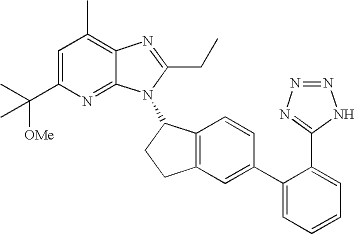

A mixture of (S)-3-(5-bromo-2,3-dihydro-1H-inden-1-yl)-2-ethyl-5-(2-methoxypropan-2-yl)-7-methyl-3H-imidazo[4,5-b]pyridine (0.10 g, 0.24 mmol), 2-(1-trityl-1H-tetrazol-5-yl)phenylboronic acid (0.16 g, 0.39 mmol), Pd(OAc)₂ (11 mg, 0.05 mmol), PPh₃ (46 mg, 0.19 mmol) and K₂CO₃ (83 mg, 0.60 mmol) was degassed for 10 min with N₂. The resulting mixture was heated at 90° C. in a sealed tube for 16 h, cooled to RT, concentrated and purified by chromatography to give the coupled product. This was refluxed in MeOH (5 mL) for 16 h and concentrated and the residue was purified by silica gel chromatography to give 3-((1S)-5-(2-(1H-tetrazol-5-yl)phenyl)-2,3-dihydro-1H-inden-1-yl)-2-ethyl-5-(2-methoxypropan-2-yl)-7-methyl-3H-imidazo[4,5-b]pyridine (55 mg, 44% over two steps). ¹HNMR (400 MHz, CDCl₃) δ ppm 8.00 (br s, 1H), 7.60-7.40 (m, 3H), 7.20-7.00 (m, 2H), 6.90 (brs, 1H), 6.77 (br s, 1H), 3.25 (m, 1H), 3.10-2.40 (m, 11H), 1.60-1.20 (m, 9H). HPLC: 97.92%. MS: 494.03 (M⁺+1).

### Example 18

### 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-4-methylpentan-2-ol

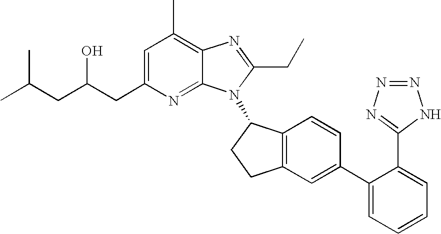

### Steps 1 and 2. 1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)-4-methylpentan-2-ol

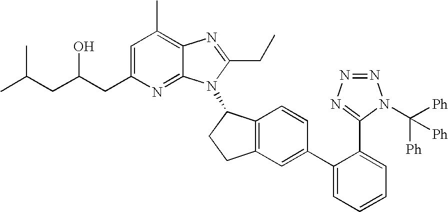

A solution of 5-allyl-2-ethyl-7-methyl-3-{(S)-5-[2-(1-trityl-1H-tetrazol-5-yl)-phenyl]-indan-1-yl}-3H-imidazo[4,5-b]pyridine (2 g, 2.84 mmol) in dichloromethane (25 mL) and methanol (25 mL) was cooled to −50 C and ozone (g) (O3/O2 generated with Delzone-LG-7, with full power, was bubbled through the reaction mixture with a flow rate of 1 L/min for 20 min. The solution turned purple. The solvent was removed to give the ozonide intermediate (2.3 g, 110% yield). MS: M+1 754

A solution of the crude ozonide (0.8 g, 1.1 mmol) in THF (5 mL) was treated with 2 M isobutyl magnesium chloride in THF (2 mL) and stirred at room temperature for 2 h. The reaction was quenched with saturated NaHCO₃ (2 mL) and EtOAc (10 mL). The resulting organic residue was purified on silica gel using 15-75% EtOAc in hexanes as eluant to give 1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)-4-methylpentan-2-ol (330 mg, 41% yield) as a gum.

### Step 3. 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-4-methyl pentan-2-ol

(0110297-065). A solution of 1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)-4-methylpentan-2-ol (330 mg, 0.43 mmol) in methanol (15 mL) was refluxed for 3 h. After removal of solvent, the residue was purified via silica gel chromatography using 1-5% MeOH in dichloromethane as eluant to give 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-4-methylpentan-2-ol (140 mg, 62% yield) as a light colored solid. ¹H NMR (400 MHz, DMSO-d6) δ ppm 0.69-0.82 (m) 0.97-1.14 (m) 1.29 (d, J=1.36 Hz) 1.62-1.75 (m) 2.52 (d, J=1.75 Hz) 2.64-2.77 (m) 2.78-2.92 (m) 2.94-3.08 (m) 3.25 (br. s.) 3.84 (d, J=21.25 Hz) 5.77 (s) 6.32 (br. s) 6.70-6.83 (m) 6.89 (br. s.) 7.15 (d, J=7.99 Hz) 7.58 (dd, J=11.79, 7.12 Hz) 7.64-7.72 (m). MS: M+1 522

Examples 19, 20 and 21 were prepared by analogous methodology as Example 18 using the appropriate Grignard reagent in Step 2.

### Example 19

### 2 (3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-1-cyclopropylethanol

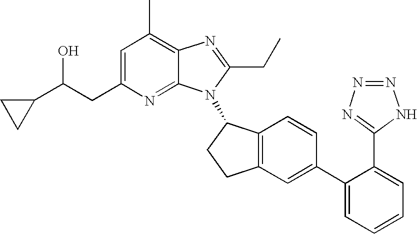

1H NMR (400 MHz, DMSO-d6) δ ppm −0.22 (br. s., 1H) −0.08 (br. s., 1H) 0.01-0.27 (m, 3H) 0.73 (br. s., 1H) 1.28 (br. s., 3H) 2.48 (s, 3H) 2.68 (d, J=7.02 Hz, 2H) 2.83 (br. s., 2H) 2.92-3.06 (m, 2H) 3.16 (br. s., 2H) 5.75 (s, 1H) 6.29 (br. s., 1H) 6.70-6.81 (m, 2H) 6.89 (br. s., 1H) 7.12 (d, J=6.43 Hz, 1H) 7.50-7.61 (m, 2H) 7.63-7.72 (m, 2H). MS: M+1 506. HPLC: XTerra RP18 5 uM, 4.6×250 mm column, 90:10 to 10:90, 0.1% TFA acetonitrile, linear gradient over 20 min at 1.6 mL/min; Retention time=10.9 min.

### Example 20

### 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)pent-4-en-2-ol

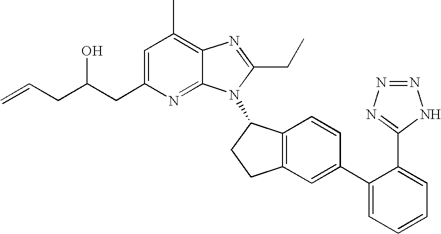

1H NMR (400 MHz, DMSO-d6) δ ppm 1.27 (t, J=6.43 Hz, 3H) 2.05 (br. s., 2H) 2.48 (s, 3H) 2.71 (d, J=31.19 Hz, 4H) 2.92-3.05 (m, 2H) 3.23 (br. s., 2H) 3.79 (br. s., 1H) 4.93 (d, J=15.40 Hz, 2H) 5.76 (dd, J=17.74, 12.87 Hz, 2H) 6.31 (br. s., 1H) 6.68-6.81 (m, 2H) 6.87 (s, 1H) 7.13 (d, J=6.24 Hz, 1H) 7.51-7.60 (m, 2H) 7.61-7.72 (m, 2H). MS: M+1 506. HPLC: XTerra RP18 5 uM, 4.6×250 mm column, 90:10 to 10:90, 0.1% TFA acetonitrile, linear gradient over 20 min at 1.6 mL/min; Retention time=11 min.

### Example 21

### 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-3-methylbutane-2-ol

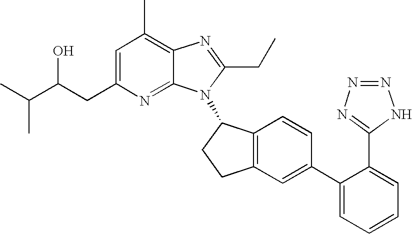

1H NMR (400 MHz, DMSO-d6) δ ppm 0.54-0.67 (m) 0.72-0.83 (m) 1.22-1.34 (m) 2.48 (s) 2.60-2.71 (m) 2.72-2.84 (m) 2.94-3.07 (m) 3.51 (br. s.) 3.80 (s,) 4.22 (s) 6.30 (br. s.) 6.67-6.81 (m) 6.89 (s) 7.14 (br. s.) 7.52-7.60 (m) 7.62-7.71 (m). MS: M+1 508 HPLC: XTerra RP18 5 uM, 4.6×250 mm column, 90:10 to 10:90, 0.1% TFA acetonitrile, linear gradient over 20 min at 1.6 mL/min; Retention time=10.3 min.

### Example 22

### 3-((1S)-5-(2-(1H-tetrazol-5-yl)phenyl)-2,3-dihydro-1H-inden-1-yl)-5-(3,3-dimethyloxiran-2-yl)-2-ethyl-7-methyl-3H-imidazo[4,5-b]pyridine

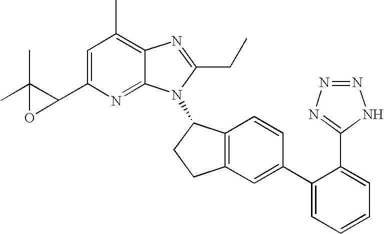

### Steps 1-5. 2-ethyl-7-methyl-5-(2-methylprop-1-enyl)-3-((1S)-5-(2-(1-trityl-1H-tetrazol-5-yl)phenyl)-2,3-dihydro-1H-inden-1-yl)-3H-imidazo[4,5-b]pyridine

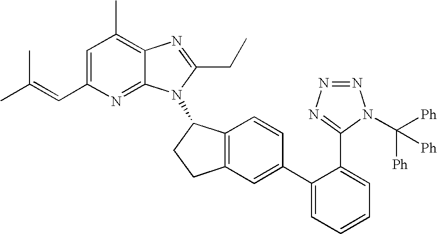

A mixture of N-(6-bromo-2-chloro-4-methylpyridin-3-yl)propionamide (3 g, 10.8 mmol) and 1,3-diphenylphosphinopropyl nickel (II) chloride (1.17 g, 2.16 mmol) in THF (10 mL) was treated at room temperature with 0.5 M 2-methylpropenyl magnesium bromide (48 mL). The reaction mixture was heated at 50 C for 16 h, cooled and quenched with saturated ammonium chloride (30 mL). The organic layer was concentrated and the residue purified on silica gel using 10-65% EtOAc in hexanes as eluant to give N-(2-chloro-4-methyl-6-(2-methylprop-1-enyl)pyridin-3-yl)propionamide (0.9 g, 33% yield) as a light colored solid. 1H-NMR (CDCl₃) MS: M+1 253, 255

N-(2-chloro-4-methyl-6-(2-methylprop-1-enyl)pyridin-3-yl)propionamide was elaborated in an analogous manner in several steps as described in Example 9 to give 2-ethyl-7-methyl-5-(2-methylprop-1-enyl)-3-((1S)-5-(2-(1-trityl-1H-tetrazol-5-yl)phenyl)-2,3-dihydro-1H-inden-1-yl)-3H-imidazo[4,5-b]pyridine.

### Step 6 and 7

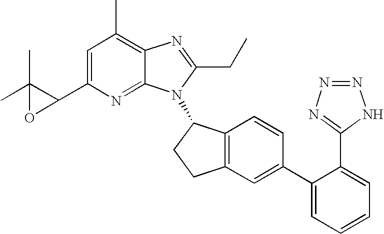

(0511783-020) A mixture of 2-ethyl-7-methyl-5-(2-methylprop-1-enyl)-3-((1S)-5-(2-(1-trityl-1H-tetrazol-5-yl)phenyl)-2,3-dihydro-1H-inden-1-yl)-3H-imidazo[4,5-b]pyridine (1.2 g, 1.67 mmol) and m-chloroperbenzoic acid (0.47 g, 2.09 mmol) in dichloromethane (5 mL) was stirred at room temperature overnight. The reaction mixture was purified on silica gel using 10-55% EtOAc in hexanes to give 5-(3,3-dimethyloxiran-2-yl)-2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridine (0.9 g, 73% yield) as a foam. (0511783-028) A solution of the epoxidation product (0.25 g, 0.34 mmol) in THF (15 mL) and MeOH (0.5 mL) was treated with trimethylsilyl chloride (5 drops) and stirred for 30 min and quenched with sodium bicarbonate (2 g), and then 5 drops of water. After stirring 30 min, the solvent was removed and the crude product purified on silica gel using 0-10% MeOH in dichloromethane to give 3-((1S)-5-(2-(1H-tetrazol-5-yl)phenyl)-2,3-dihydro-1H-inden-1-yl)-5-(3,3-dimethyloxiran-2-yl)-2-ethyl-7-methyl-3H-imidazo[4,5-b]pyridine (45 mg, 27% yield) as a solid. 1H NMR (400 MHz, DMSO-d6) δ ppm 0.67 (br. s., 1H) 0.78 (br. s., 1H) 0.88 (br. s., 1H) 0.99 (d, J=12.30 Hz, 4H) 1.17-1.37 (m, 8H) 1.41 (s, 3H) 2.53 (d, J=3.71 Hz, 6H) 2.67 (br. s., 4H) 3.02 (br. s., 7H) 3.08 (d, J=28.50 Hz, 1H) 3.88 (d, J=23.62 Hz, 1H) 4.42 (br. s., 1H) 6.24 (br. s., 2H) 6.68-6.83 (m, 4H) 6.93 (d, J=5.08 Hz, 1H) 7.03-7.20 (m, 4H) 7.50-7.60 (m, 4H) 7.61-7.72 (m, 4H). MS: M+1 492

### Example 23

### 3-((1S)-5-(2-(1H-tetrazol-5-yl)phenyl)-2,3-dihydro-1H-inden-1-yl)-2-ethyl-5-((E)-3-methoxyprop-1-enyl)-7-methyl-3H-imidazo[4,5-b]pyridine

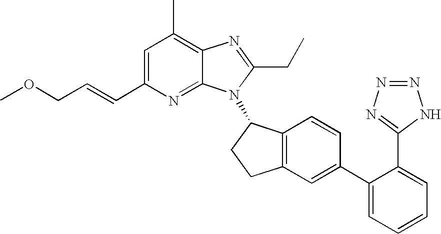

### Step 1. (E)-N-(2-chloro-6-(3-methoxyprop-1-enyl)-4-methylpyridin-3-yl)propionamide

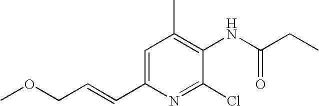

(0511315-033) A mixture of N-(6-bromo-2-chloro-4-methylpyridin-3-yl)propionamide (1.0 g, 3.6 mmol), (E)-2-(3-methoxyprop-1-enyl)-4,4,5,5-tetramethyl-1,3,2-dioxaborolane (1.07 g, 5.4 mmol), Pd (II) acetate (81 mg, 0.36 mmol), triphenylphosphine (380 mg, 1.44 mmol) and potassium carbonate (1.62 g, 11.7 mmol) in DME (5 mL) was bubbled with N2 (g) for 10 min. Water (110 mg, 16.6 mmol) was added and N2 (g) continued bubbling for 20 min. The reaction mixture was heated at 80 C for 16 h, cooled and filtered and the reaction solution purified on silica gel using 5-75% EtOAc in hexanes as eluant to give (E)-N-(2-chloro-6-(3-methoxyprop-1-enyl)-4-methylpyridin-3-yl)propionamide (0.65 g, 67% yield) as a solid. MS: M+1 269, 271

Steps 2-6. (E)-N-(2-chloro-6-(3-methoxyprop-1-enyl)-4-methylpyridin-3-yl)propionamide was elaborated in an analogous manner via the tandem Suzuki/cyclization procedure in several steps as described in Example 9 and purified on neutral alumina with 0-5% MeOH in dichloromethane as eluant to give 3-((1S)-5-(2-(1H-tetrazol-5-yl)phenyl)-2,3-dihydro-1H-inden-1-yl)-2-ethyl-5-((E)-3-methoxyprop-1-enyl)-7-methyl-3H-imidazo[4,5-b]pyridine (0.45 g) as a light colored solid. 1H NMR (400 MHz, CHLOROFORM-d) δ ppm 1.46 (none, 1H) 1.46 (t, J=7.02 Hz, 3H) 2.66 (s, 3H) 2.71 (br. s., 1H) 2.79 (br. s., 2H) 2.96-3.18 (m, 3H) 3.33 (s, 3H) 3.38 (d, J=6.04 Hz, 2H) 6.62 (br. s., 3H) 6.66 (br. s., 1H) 6.81 (d, J=7.60 Hz, 1H) 6.86-6.92 (m, 1H) 7.28 (s, 1H) 7.45-7.55 (m, 2H) 7.59 (t, J=7.51 Hz, 1H) 7.87 (d, J=7.60 Hz, 1H). MS: M+1 492

### Example 24

### 3-((1S)-5-(2-(1H-tetrazol-5-yl)phenyl)-2,3-dihydro-1H-inden-1-yl)-2-ethyl-7-methyl-5-(2-(pyridin-3-yl)ethyl)-3H-imidazo[4,5-b]pyridine

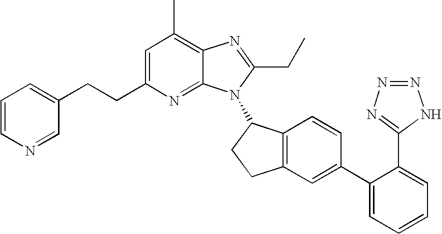

### Step 1. 2-ethyl-7-methyl-5-(2-(pyridin-3-yl)ethynyl)-3-((1S)-5-(2-(1-trityl-1H-tetrazol-5-yl)phenyl)-2,3-dihydro-1H-inden-1-yl)-3H-imidazo[4,5-b]pyridine

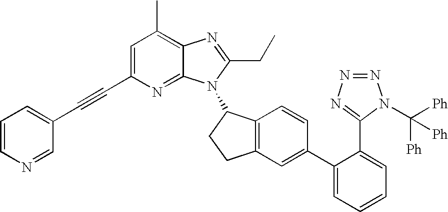

A solution of 5-bromo-2-ethyl-7-methyl-3-{(S)-5-[2-(1-trityl-1H-tetrazol-5-yl)-phenyl]-indan-1-yl}-3H-imidazo[4,5-b]pyridine (0.175 g, 0.236 mmol) in anhydrous THF (0.6 mL) and Et₃N (0.15 mL) in a vial was treated with PdCl₂(PPh₃)₂ (0.0165 g, 0.024 mmol) and CuI (0.009 g, 0.047 mmol). 3-ethynyl pyridine (0.026 mL, 0.259 mmol) was added and nitrogen blown over vial. The vial was sealed and heated at 55° C. for 6 h. Product formation was confirmed by MS. Mixture allowed to cool, diluted with EtOAc, filtered through celite, washed with EtOAc and solvents removed. Residue purified by MPLC on silica gel eluting with ethyl acetate in hexanes (0 to 45%). Pure fractions were combined and solvent removed. The residue was dried on high vacuum overnight. 2-ethyl-7-methyl-5-(2-(pyridin-3-yl)ethynyl)-3-((1S)-5-(2-(1-trityl-1H-tetrazol-5-yl)phenyl)-2,3-dihydro-1H-inden-1-yl)-3H-imidazo[4,5-b]pyridine was obtained as an off-white solid (0.138 g, 77% yield): 521.1 (APCI)⁻

### Step 2. 3-((1S)-5-(2-(1H-tetrazol-5-yl)phenyl)-2,3-dihydro-1H-inden-1-yl)-2-ethyl-7-methyl-5-(2-(pyridin-3-yl)ethynyl)-3H-imidazo[4,5-b]pyridine

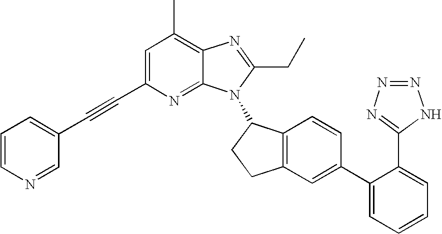

A solution of 2-ethyl-7-methyl-5-(2-(pyridin-3-yl)ethynyl)-3-((1S)-5-(2-(1-trityl-1H-tetrazol-5-yl)phenyl)-2,3-dihydro-1H-inden-1-yl)-3H-imidazo[4,5-b]pyridine (0.138 g, 0.180 mmol) in MeOH was refluxed for 24 hrs. The reaction mixture was concentrated and the residue purified by MPLC on silica gel eluting with MeOH in dichloromethane (0-8%). The pure fractions were concentrated and dried. 3-((1S)-5-(2-(1H-tetrazol-5-yl)phenyl)-2,3-dihydro-1H-inden-1-yl)-2-ethyl-7-methyl-5-(2-(pyridin-3-yl)ethynyl)-3H-imidazo[4,5-b]pyridine was obtained as an off-white solid (0.062 g, 66%). ¹H NMR (DMSO d-6) consistent with desired product; (0.062 g, 66%). 523.1 (APCI)⁺; 522.1 (APCI)⁻

### Step 3. 3-((1S)-5-(2-(1H-tetrazol-5-yl)phenyl)-2,3-dihydro-1H-inden-1-yl)-2-ethyl-7-methyl-5-(2-(pyridin-3-yl)ethyl)-3H-imidazo[4,5-b]pyridine

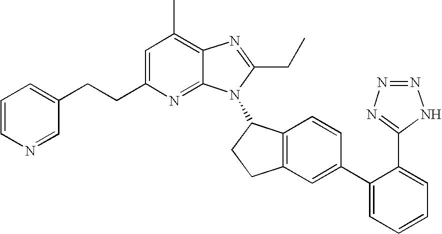

3-((1S)-5-(2-(1H-tetrazol-5-yl)phenyl)-2,3-dihydro-1H-inden-1-yl)-2-ethyl-7-methyl-5-(2-(pyridin-3-yl)ethynyl)-3H-imidazo[4,5-b]pyridine (0.045 g, 0.086 mmol) was hydrogenated over 5% Pd/C (0.03 g) in 1:1 MeOH/THF (16 mL) for 2 h. The solvent was removed and the residue was loaded into column for purification by MPLC in silica gel eluting with MeOH in DCM (0 to 6%) to obtain 3-((1S)-5-(2-(1H-tetrazol-5-yl)phenyl)-2,3-dihydro-1H-inden-1-yl)-2-ethyl-7-methyl-5-(2-(pyridin-3-yl)ethyl)-3H-imidazo[4,5-b]pyridine as a white solid after drying (0.028 g, 63%). ¹H NMR (400 MHz, DMSO-de) δ ppm 1.28 (t, J=7.02 Hz, 3H), 2.44 (s, 3H), 2.62-2.73 (m, 2H), 2.77-3.09 (m, 7H), 6.31 (s, 1H), 6.73-6.81 (m, 2H), 6.82 (bs, 1H), 7.15 (s, 1H), 7.18 (d, J=4.68 Hz, 1H), 7.36-7.48 (m, 2H), 7.49-7.66 (m, 3H), 8.19 (bs, 1H), 8.29 (d, J=6.24 Hz, 1H); CIMS: 526.1 (APCI)+, 524.2 (APCI)−; HPLC: 98.5% purity; Rt=18.406 min, method A.

Examples 25-37, Examples 40-59 and Examples 68-69 were prepared via analogous methodology as described in Example 24 using the appropriate alkyne in Step 1. Selected examples used an alternative sequence whereby the Sonogoshira coupling of the alkyne and the substituted heterocycle was performed prior to installation of the tetrazolylphenyl moiety.

### Example 25

### 3-((1S)-5-(2-(1H-tetrazol-5-yl)phenyl)-2,3-dihydro-1H-inden-1-yl)-2-ethyl-7-methyl-5-(2-(pyridin-2-yl)ethyl)-3H-imidazo[4,5-b]pyridine

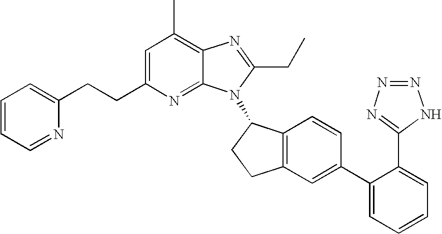

2-Ethynylpyridine was used as the alkyne according to the method of Example 24. ¹H NMR (400 MHz, DMSO-d₆) δ ppm 1.26 (t, J=7.21 Hz, 3H), 2.45 (s, 3H), 2.51-2.53 (m, 2H), 2.59-2.73 (m, 2H), 2.80 (bs, 2H), 2.94-3.13 (m, 6H), 6.31 (bs, 1H), 6.69-6.75 (m, 1H), 6.74-6.80 (m, 1H), 6.86 (s, 1H), 7.02 (bs, 1H), 7.13 (d, J=7.41 Hz, 1H), 7.16 (s, 1H), 7.48 (d, J=7.80 Hz, 1H), 7.51-7.67 (m, 3H), 8.43 (d, J=3.90 Hz, 1H); CIMS: 527.4 (APCI)+, 525.7 (APCI)−; HPLC: 96.79% purity; Rt=10.360 min, method A.

### Example 26

### 3-((1S)-5-(2-(1H-tetrazol-5-yl)phenyl)-2,3-dihydro-1H-inden-1-yl)-5-(2-fluorophenethyl)-2-ethyl-7-methyl-3H-imidazo[4,5-b]pyridine

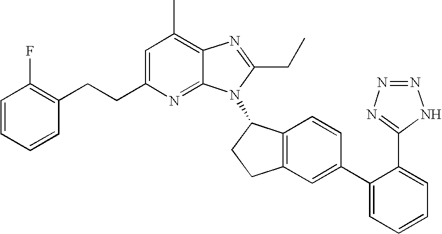

2-Fluorophenylacetylene was used as the alkyne according to the method of Example 24.

### Example 27

### 3-((1S)-5-(2-(1H-tetrazol-5-yl)phenyl)-2,3-dihydro-1H-inden-1-yl)-5-(4-methoxyphenethyl)-2-ethyl-7-methyl-3H-imidazo[4,5-b]pyridine

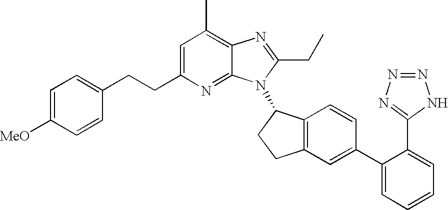

1-Ethynyl-4-methoxybenzene was used as the alkyne according to the method of Example 24. ¹H NMR (400 MHz, DMSO-d₆) δ ppm 1.28 (t, J=7.02 Hz, 3H), 2.44 (s, 3H), 2.62-2.73 (m, 2H), 2.77-3.09 (m, 7H), 6.31 (s, 1H), 6.73-6.81 (m, 2H), 6.82 (bs, 1H), 7.15 (s, 1H), 7.18 (d, J=4.68 Hz, 1H), 7.36-7.48 (m, 2H), 7.49-7.66 (m, 3H), 8.19 (bs, 1H), 8.29 (d, J=6.24 Hz, 1H); CIMS: 556.2 (APCI)+, 554.2 (APCI)−; HPLC: >99% purity; Rt=17.673 min, method A.

### Example 28

### ((1S)-5-(2-(1H-tetrazol-5-yl)phenyl)-2,3-dihydro-1H-inden-1-yl)-5-(2-methylphenethyl)-2-ethyl-7-methyl-3H-imidazo[4,5-b]pyridine

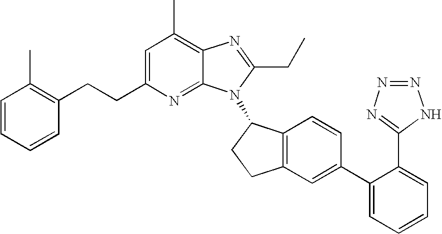

2-Methylphenylacetylene was used as the alkyne according to the method of Example 24.

### Example 29

### 3-((1S)-5-(2-(1H-tetrazol-5-yl)phenyl)-2,3-dihydro-1H-inden-1-yl)-5-(3-methoxyphenethyl)-2-ethyl-7-methyl-3H-imidazo[4,5-b]pyridine

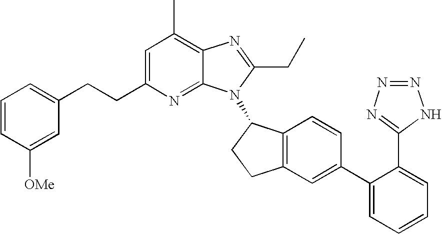

1-Ethynyl-3-methoxybenzene was used as the alkyne according to the method of Example 24. ¹H NMR (400 MHz, DMSO-d₆) δ ppm 1.26 (t, J=7.41 Hz, 3H), 2.46 (s, 3H), 2.63-2.73 (m, 2H), 2.75-3.08 (m, 6H), 3.66 (s, 3H), 6.34 (bs, 1H), 6.62-6.72 (m, 2H), 6.73-6.83 (m, 2H), 6.87 (bs, 1H), 7.06-7.14 (m, 1H), 7.15 (s, 1H), 7.46 (d, J=7.02 Hz, 1H), 7.51-7.70 (m, 4H); CIMS: 556.2 (APCI)+, 554.2 (APCI)−; HPLC: >99% purity; Rt=17.678 min, method A.

### Example 30

### 3-((1S)-5-(2-(1H-tetrazol-5-yl)phenyl)-2,3-dihydro-1H-inden-1-yl)-2-ethyl-7-methyl-5-phenethyl-3H-imidazo[4,5-b]pyridine

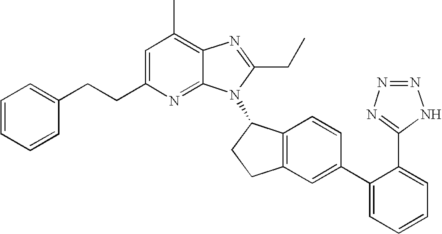

1-Ethynylbenzene was used as the alkyne according to the method of Example 24.

### Example 31

### 3-((1S)-5-(2-(1H-tetrazol-5-yl)phenyl)-2,3-dihydro-1H-inden-1-yl)-5-(2,5-dimethylphenethyl)-2-ethyl-7-methyl-3H-imidazo[4,5-b]pyridine

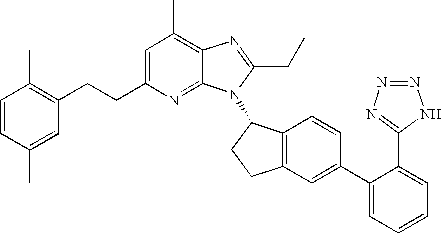

2-Ethynyl-1,4-dimethylbenzene was used as the alkyne according to the method of Example 24.

### Example 32

### 3-((1S)-5-(2-(1H-tetrazol-5-yl)phenyl)-2,3-dihydro-1H-inden-1-yl)-2-ethyl-7-methyl-5-(3-(pyridin-3-yl)propyl)-3H-imidazo[4,5-b]pyridine

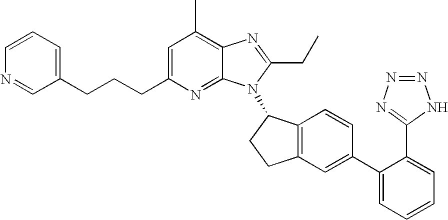

1-(Pyridin-3-yl)prop-2-yn-1-ol was used as the alkyne according to the method of Example 24. ¹H NMR (400 MHz, DMSO-d₆) δ ppm 1.13-1.33 (m, 3H), 1.78-1.94 (m, 2H), 2.48 (s, 3H), 2.51-2.56 (m, 1H), 2.56-2.90 (m, 4H), 2.92-3.05 (m, 1H), 6.30 (bs, 1H), 6.69-6.79 (m, 2H), 6.87 (s, 1H), 7.09 (s, 1H), 7.24 (dd, J=7.41, 4.68 Hz, 1H), 7.37 (d, J=6.24 Hz, 1H), 7.50-7.59 (m, 2H), 7.59-7.70 (m, 2H), 8.36 (bs, 2H); CIMS: 541.2 (APCI)+, 539.2 (APCI)−; HPLC: 94.48% purity; Rt=8.301 min; method A.

### Example 33

### 5-(3-(1H-pyrazol-1-yl)propyl)-3-((1S)-5-(2-(1H-tetrazol-5-yl)phenyl)-2,3-dihydro-1H-inden-1-yl)-2-ethyl-7-methyl-3H-imidazo[4,5-b]pyridine

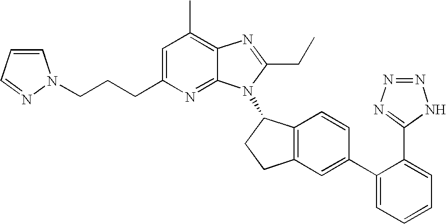

### Step 1. 1-(prop-2-ynyl)-1H-pyrazole

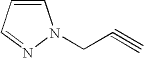

To a solution of pyrazole (5.0 g, 71.7 mmol) in dichloromethane (100 mL) was added propargyl bromide (12.0 mL, 107.6 mmol, 80% solution in toluene) and tetrabutylammonium bromide (0.5 g, 1.5 mmol). The mixture was cooled to 0° C. and aqueous sodium hydroxide (15 mL, 50%) was introduced. It was then stirred for 2 h and diluted with dichloromethane (100 mL). Organics were separated, washed with water (3×50 mL), dried and evaporated. The resulting crude material was purified by column chromatography (5%˜10% ethyl acetate in hexanes) to obtain 1-(prop-2-ynyl)-1H-pyrazole (2.3 g, 30%) as an oil. ¹H NMR (400 MHz, CDCl₃): δ 7.65-7.55 (m, 2H), 6.30-6.25 (m, 1H), 6.00-5.95 (m, 2H), 2.55-2.50 (m, 1H).

1-(Prop-2-ynyl)-1H-pyrazole was used as the alkyne according to the method of Example 24. mp 138-139° C.; ¹H NMR (400 MHz, CDCl₃) δ ppm 7.80-7.70 (m, 1H), 7.60-7.40 (m, 2H), 7.40-7.30 (m, 2H), 6.95 (s, 1H), 6.85 (bs, 1H), 6.80 (s, 1H), 6.60 (s, 2H), 6.15 (s, 1H), 5.85 (bs, 1H), 4.05-3.95 (m, 2H), 3.20-2.90 (m, 4H), 2.90-2.65 (m, 2H), 2.55 (s, 3H), 2.30-2.00 (m, 2H), 1.45 (t, 2H), 1.40-1.20 (m, 2H), 0.95-0.80 (m, 2H); LRMS (ES⁺) 530.04; HPLC: 97.22% purity.

### Example 34

### 3-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-1-(pyridin-3-yl)propan-1-ol

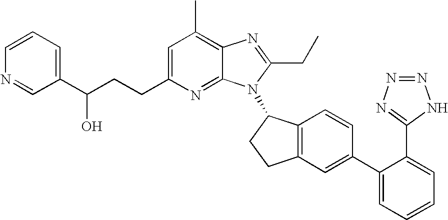

1-(Pyridin-3-yl)prop-2-yn-1-ol was used as the alkyne according to the method of Example 24. ¹H NMR (400 MHz, DMSO-d₆) δ ppm 1.16-1.33 (m, 3H), 1.80-2.04 (m, 2H), 2.47 (s, 3H), 2.61-2.88 (m, 4H), 2.91-3.06 (m, 1H), 4.61 (t, J=6.43 Hz, 1H), 5.36 (bs, 1H), 6.31 (bs, 1H), 6.68-6.78 (m, 2H), 6.85 (bs, 1H), 7.10 (bs, 1H), 7.19-7.33 (m, 1H), 7.33-7.50 (m, 1H), 7.51-7.73 (m, 4H), 8.32-8.54 (m, 2H); CIMS: 557.2 (APCI)+, 555.2 (APCI)−; HPLC: 77.08% purity; Rt=7.823 min; method A. Impurity was PF-04347358.

### Example 35

### 4-(3-((1S)-5-(2-(1H-tetrazol-5-yl)phenyl)-2,3-dihydro-1H-inden-1-yl)-7-methyl-2-propyl-3H-imidazo[4,5-b]pyridin-5-yl)-2-methylbutan-2-ol

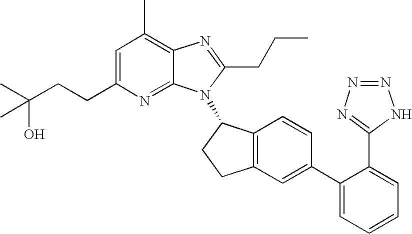

2-Methylbut-3-yn-2-ol was used as the alkyne according to the method of Example 24. 1H NMR (400 MHz, CHLOROFORM-d) δ ppm 0.93 (s, 3H) 1.11 (t, J=7.41 Hz, 3H) 1.19 (s, 3H) 1.77-2.05 (m, 4H) 2.65 (s, 3H) 2.67-2.72 (m, 1H) 2.93 (dd, J=16.67, 9.65 Hz, 1H) 3.02-3.30 (m, 5H) 3.33-3.45 (m, 1H) 5.31 (s, 1H) 6.00 (t, J=8.58 Hz, 1H) 6.63 (s, 2H) 6.91 (s, 1H) 7.49-7.56 (m, 3H) 7.58-7.66 (m, 1H) 8.13 (d, J=8.19 Hz, 1H).

### Example 36

### (S)-4-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)butan-2-ol

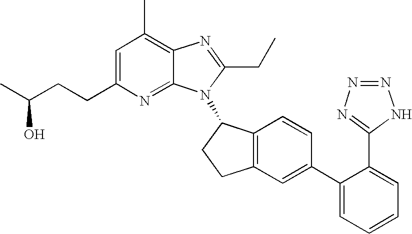

### Step 1. (S)-4-(2-Ethyl-7-methyl-3-{5-[2-(1-trityl-1H-tetrazol-5-yl)-phenyl]-indan-1-yl}-3H-imidazo[4,5-b]pyridin-5-yl)-but-3-yn-2-ol

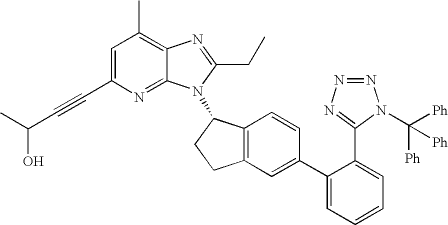

5-Bromo-2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridine (3.00 g, 4.04 mmol) was placed in vial and dissolved in anhydrous THF (6.4 mL) and Et₃N (1.6 mL). PdCl₂(PPh₃)₂ (0.284 g, 0.404 mmol) and CuI (0.154 g, 0.808 mmol) were added. 3-butyn-2-ol (0.633 mL, 8.08 mmol) was added and nitrogen blown over vial. Vial was sealed and heated at 55° C. for 24 h. Product formation was confirmed by MS. Mixture allowed to cool, diluted with EtOAc, filtered through celite, washed with EtOAc and solvents removed. Residue purified by MPLC on silica gel eluting with ethyl acetate in hexanes (0 to 45%). Pure fractions were combined and solvent removed. Residue dried high vacuum overnight. (S)-4-(2-Ethyl-7-methyl-3-{5-[2-(1-trityl-1H-tetrazol-5-yl)-phenyl]-indan-1-yl}-3H-imidazo[4,5-b]pyridin-5-yl)-but-3-yn-2-ol was obtained as a light yellowish solid (2.45 g, 83% yield): CIMS 732.2 (APCI)+; 488.1 (APCI)−; HPLC: 99.31% purity; Rt=21.033 min; method A.

Step 2. (S,S)-4-(2-Ethyl-7-methyl-3-(5-[2-(1H-tetrazol-5-yl)-phenyl]-indan-1-yl)-3H-imidazo[4,5-b]pyridin-5-yl)-butan-2-ol (PF-04029829). (S)-4-(2-Ethyl-7-methyl-3-{5-[2-(1-trityl-1H-tetrazol-5-yl)-phenyl]-indan-1-yl}-3H-imidazo[4,5-b]pyridin-5-yl)-but-3-yn-2-ol (2.43 g, 3.32 mmol) was hydrogenated over 10% Pd/C (0.5 g) in 1:1 MeOH/THF (180 mL) for 17 h. Catalyst was filtered off and the solvent was removed. Residue was dissolved in MeOH (100 mL) and the solution heated at 65° C. for 24 h. The reaction mixture was concentrated and residue was loaded into column for purification by MPLC in silica gel eluting with isopropanol in DCM (0 to 4%). This purification afforded two products. The fastest moving band from the chromatographic purification was collected and the solvent removed. Residue dried under high vacuum. This compound was registered as PF-04029829 and obtained as a white solid (0.672 g, 41%): ¹H NMR (400 MHz, DMSO-d₆) □ ppm 1.02 (d, J=6.24 Hz, 3H), 1.25 (t, J=6.82 Hz, 3H), 1.56-1.70 (m, 2H), 2.48 (s, 3H), 2.52-2.61 (m, 1H), 2.62-2.88 (m, 5H), 2.94-3.08 (m, 1H), 3.51-3.63 (m, 1H), 4.41 (bs, 1H), 6.33 (bs, 1H), 6.69-6.82 (m, 2H), 6.87 (s, 1H), 7.14 (s, 1H), 7.52-7.61 (m, 2H), 7.62-7.71 (m, 2H); CIMS: 494.3 (APCI)+; 492.4 (APCI)−; HPLC: >99% purity; Rt=9.831 min; method A.

### Example 37

### (R)-4-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)butan-2-ol

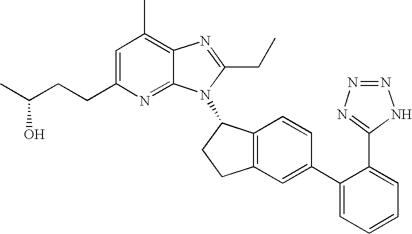

(R)-4-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)butan-2-ol. The slower moving band from Example 36, Step 2 was also collected and solvent removed. (R)-4-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)butan-2-ol was obtained as an off-white solid (0.469 g, 29%). ¹H NMR (400 MHz, DMSO-d₆) δ ppm 1.00 (d, J=6.24 Hz, 3H), 1.25 (t, J=6.82 Hz, 3H), 1.53-1.69 (m, 2H), 2.47 (s, 3H), 2.51-2.61 (m, 1H), 2.60-2.89 (m, 5H), 2.94-3.07 (m, 1H), 3.51 (dd, J=10.53, 4.68 Hz, 1H), 4.38 (bs, 1H), 6.33 (bs, 1H), 6.68-6.79 (m, 2H), 6.87 (s, 1H), 7.15 (s, 1H), 7.52-7.60 (m, 2H), 7.61-7.71 (m, 2H); CIMS: 494.3 (APCI)+; 492.4 (APCI)−; HPLC: >99% purity; Rt=9.816 min; method A.

### Example 38

### 3-((1S)-5-(2-(1H-tetrazol-5-yl)phenyl)-2,3-dihydro-1H-inden-1-yl)-2-ethyl-5-((S)-3-methoxybutyl)-7-methyl-3H-imidazo[4,5-b]pyridine

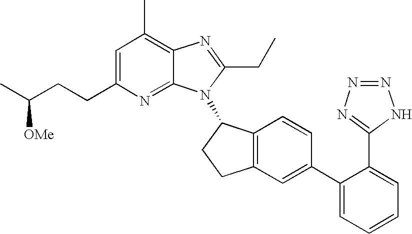

### Step 1. (S)-4-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)but-3-yn-2-ol

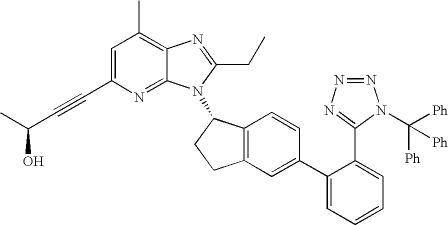

A mixture of 5-bromo-2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridine (0.24 g, 0.32 mmol), (S)-but-3-yn-2-ol (90 mg, 1.28 mmol), PdCl₂(PPh₃)₂ (11 mg, 0.02 mmol), CuI (6 mg, 0.03 mmol) and Et₃N (2 mL) in THF (8 mL) was deoxygenated by bubbling N₂ for 5 min. The resulting mixture was heated in a sealed tube at 50° C. for 16 h, cooled to RT, concentrated. The residue was purified by chromatography to give (S)-4-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)but-3-yn-2-ol (0.15 g, 64%). ¹H NMR (CDC₃, 400 MHz) δ ppm 7.96 (d, 1H), 7.60-7.10 (m, 12H), 7.05-6.90 (m, 7H), 6.70-6.50 (m, 2H), 4.65 (s, 1H), 3.20 (m, 1H), 2.80-2.20 (m, 8H), 1.80 (s, 1H), 1.60 (d, 3H), 1.20 (t, 3H).

### Step 2. 2-ethyl-5-((S)-3-methoxybut-1-ynyl)-7-methyl-3-((1S)-5-(2-(1-trityl-1H-tetrazol-5-yl)phenyl)-2,3-dihydro-1H-inden-1-yl)-3H-imidazo[4,5-b]pyridine

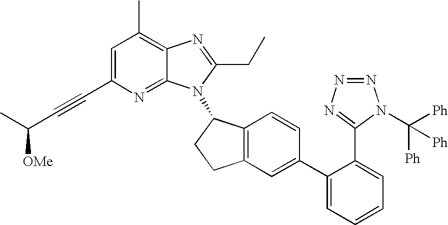

A cooled solution of (S)-4-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)but-3-yn-2-ol (0.15 g, 0.21 mmol) in THF was treated with sodium hydride (8 mg, 95%, 0.31 mmol) and methyl iodide (0.15 g, 1.03 mmol). The mixture was stirred at RT for 16 h. The reaction mixture was passed through a pad of celite. The filtrate was concentrated to give 2-ethyl-5-((S)-3-methoxybut-1-ynyl)-7-methyl-3-((1S)-5-(2-(1-trityl-1H-tetrazol-5-yl)phenyl)-2,3-dihydro-1H-inden-1-yl)-3H-imidazo[4,5-b]pyridine (0.11 g, 75%). ¹H NMR (CDCl₃, 400 MHz) δ ppm 7.96 (d, 1H), 7.60-7.10 (m, 12H), 7.05-6.90 (m, 7H), 6.70-6.50 (m, 2H), 3.80 (m, 1H), 3.50 (s, 3H), 3.20 (m, 1H), 2.80-2.20 (m, 8H), 1.60 (d, 3H), 1.20 (t, 3H).

### Step 3. 3-((1S)-5-(2-(1H-tetrazol-5-yl)phenyl)-2,3-dihydro-1H-inden-1-yl)-2-ethyl-5-((S)-3-methoxybutyl)-7-methyl-3H-imidazo[4,5-b]pyridine

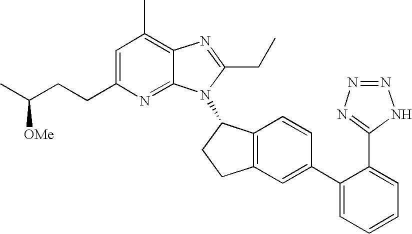

A mixture of 2-ethyl-5-((S)-3-methoxybut-1-ynyl)-7-methyl-3-((1S)-5-(2-(1-trityl-1H-tetrazol-5-yl)phenyl)-2,3-dihydro-1H-inden-1-yl)-3H-imidazo[4,5-b]pyridine (0.11 g, 0.15 mmol), HCO₂NH₄ (0.14 g, 2.2 mmol) and 10% Pd—C (80 mg) was refluxed under N₂ for 16 h, cooled to RT, filtered through a pad of celite. The filtrate was concentrated. The residue was purified by chromatography to give 3-((1S)-5-(2-(1H-tetrazol-5-yl)phenyl)-2,3-dihydro-1H-inden-1-yl)-2-ethyl-5-((S)-3-methoxybutyl)-7-methyl-3H-imidazo[4,5-b]pyridine (34 mg): ¹H NMR (CDCl₃, 400 MHz) δ ppm 7.96 (d, 1H), 7.70-7.40 (m, 3H), 7.24 (s, 1H), 6.90-6.50 (m, 3H), 6.00 (s, 1H), 3.80-2.40 (m, 15H), 2.00-1.00 (m, 8H). MS: 508.29 (M⁺+1). HPLC: 96.16%.

### Example 39

### 3-((1S)-5-(2-(1H-tetrazol-5-yl)phenyl)-2,3-dihydro-1H-inden-1-yl)-2-ethyl-5-((R)-3-methoxybutyl)-7-methyl-3H-imidazo[4,5-b]pyridine

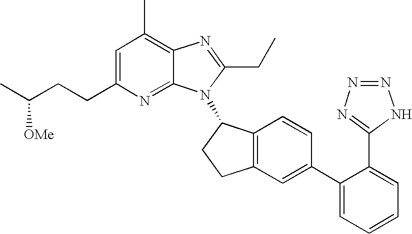

Using an analogous procedures as Example 38 (R)-but-3-yn-2-ol was used as reagent to give (R)-4-(2-ethyl-7-methyl-3-((S)-5-(2-(1-trityl-1H-tetrazol-5-yl)phenyl)-2,3-dihydro-1H-inden-1-yl)-3H-imidazo[4,5-b]pyridin-5-yl)but-3-yn-2-ol. This was followed by methylation of the alcohol, hydrogenation of the triple bond and removal of the trityl moiety to give 3-((1S)-5-(2-(1H-tetrazol-5-yl)phenyl)-2,3-dihydro-1H-inden-1-yl)-2-ethyl-5-((R)-3-methoxybutyl)-7-methyl-3H-imidazo[4,5-b]pyridine. ¹H NMR (400 MHz, CDCl₃) δ ppm 7.90 (s, 1H), 7.60-7.42 (m, 3H), 7.02-6.60 (d, 4H), 6.02 (br s, 1H), 3.40-0.95 (m, 24H). MS=508 (M+), HPLC: 91.46%.

### Example 40

### (R)-4-(3-((1S)-5-(2-(1H-tetrazol-5-yl)phenyl)-2,3-dihydro-1H-inden-1-yl)-7-methyl-2-propyl-3H-imidazo[4,5-b]pyridin-5-yl)butan-2-ol

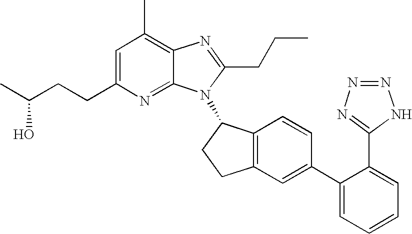

### Step 1. (S)-4-(7-methyl-2-propyl-3-((1S)-5-(2-(1-trityl-1H-tetrazol-5-yl)phenyl)-2,3-dihydro-1H-inden-1-yl)-3H-imidazo[4,5-b]pyridin-5-yl)but-3-yn-2-ol

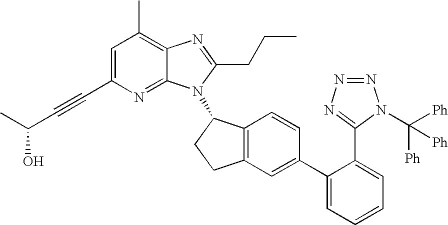

(R)-But-3-yn-2-ol was used for coupling the alkyne with 5-bromo-2-propyl-7-methyl-3-((1S)-5-(2-(1-trityl-1H-tetrazol-5-yl)phenyl)-2,3-dihydro-1H-inden-1-yl)-3H-imidazo[4,5-b]pyridine according to the method of Example 24. 1H NMR (400 MHz, CHLOROFORM-d) δ ppm 0.89 (d, J=6.04 Hz, 3H) 1.14 (t, J=7.31 Hz, 3H) 1.58 (br. s., 1H) 1.70-1.79 (m, 2H) 1.95 (dd, J=15.79, 7.41 Hz, 2H) 2.61 (br. s., 1H) 2.69 (br. s., 3H) 2.96-3.08 (m, 2H) 3.10-3.17 (m, 2H) 3.17-3.24 (m, 2H) 3.29 (d, J=4.68 Hz, 2H) 5.90-6.02 (m, 1H) 6.82 (d, J=7.80 Hz, 1H) 6.91 (br. s., 1H) 7.05 (d, J=8.97 Hz, 1H) 7.15 (s, 1H) 7.48 (dd, J=7.21, 1.56 Hz, 1H) 7.51-7.62 (m, 2H) 8.21 (dd, J=7.70, 1.27 Hz, 1H). MS APCI (+/−) 508.4/506.2

### Example 41

### (S)-4-(3-((1S)-5-(2-(1H-tetrazol-5-yl)phenyl)-2,3-dihydro-1H-inden-1-yl)-7-methyl-2-propyl-3H-imidazo[4,5-b]pyridin-5-yl)butan-2-ol

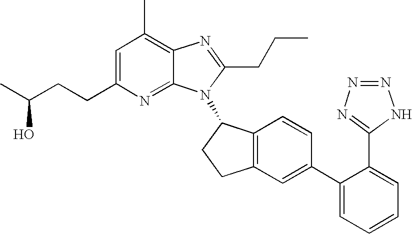

(S)-but-3-yn-2-ol was used for coupling the alkyne with 5-bromo-2-propyl-7-methyl-3-((1S)-5-(2-(1-trityl-1H-tetrazol-5-yl)phenyl)-2,3-dihydro-1H-inden-1-yl)-3H-imidazo[4,5-b]pyridine according to the method of Example 24. 1H NMR (400 MHz, CHLOROFORM-d) δ ppm 0.89 (d, J=6.43 Hz, 3H) 1.12 (t, J=7.31 Hz, 3H) 1.54-1.78 (m, 2H) 1.87-2.06 (m, 4H) 2.65 (s, 3H) 2.79-2.94 (m, 2H) 3.00-3.23 (m, 4H) 3.40 (dd, J=16.77, 10.92 Hz, 1H) 3.92-4.02 (m, 1H) 5.98 (t, J=8.97 Hz, 1H) 6.75-6.78 (m, 1H) 6.80-6.84 (m, 1H) 6.89 (s, 1H) 7.44 (s, 1H) 7.49-7.56 (m, 2H) 7.60 (t, J=6.73 Hz, 1H) 8.15 (d, J=7.60 Hz, 1H). MS APCI (+/−) 508.2/506.2

### Example 42

### 1-(2-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)ethyl)cyclopentanol

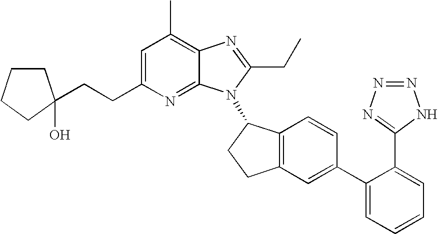

1-Ethynylcyclopentanol was used as the alkyne according to the method of Example 24. ¹H NMR (400 MHz, DMSO-d₆) δ ppm 1.24 (t, J=6.24 Hz, 3H), 1.31-1.57 (m, 6H), 1.61-1.71 (m, 2H), 1.76 (dd, J=7.31, 5.95 Hz, 2H), 2.47 (s, 3H), 2.62-2.86 (m, 5H), 2.93-3.08 (m, 2H), 4.07 (s, 1H), 6.34 (s, 1H), 6.69-6.80 (m, 2H), 6.88 (s, 1H), 7.13 (s, 1H), 7.55 (dd, J=14.23, 7.60 Hz, 2H), 7.61-7.70 (m, 2H); CIMS: 534.4 (APCI)+, 532.4 (APCI)−; HPLC: 97.47% purity; Rt=11.003 min, method A.

### Example 43

### 1-(2-(3-((1S)-5-(2-(1H-tetrazol-5-yl)phenyl)-2,3-dihydro-1H-inden-1-yl)-7-methyl-2-propyl-3H-imidazo[4,5-b]pyridin-5-yl)ethyl)cyclopentanol

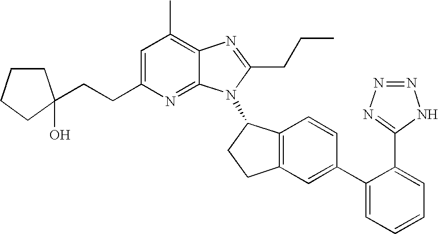

1-Ethynylcyclopentanol was used as the alkyne and coupled with 5-bromo-2-propyl-7-methyl-3-((1S)-5-(2-(1-trityl-1H-tetrazol-5-yl)phenyl)-2,3-dihydro-1H-inden-1-yl)-3H-imidazo[4,5-b]pyridine according to the method of Example 24. 1H NMR (400 MHz, CHLOROFORM-d) δ ppm 1.12 (t, J=7.31 Hz, 3H) 1.17 (d, J=8.38 Hz, 1H) 1.26 (d, J=10.53 Hz, 1H) 1.45 (br. s., 4H) 1.60 (br. s., 2H) 1.85-2.00 (m, 3H) 2.08-2.21 (m, 1H) 2.65 (s, 4H) 2.95-3.13 (m, 4H) 3.14-3.24 (m, 2H) 3.30-3.42 (m, 1H) 5.31 (s, 1H) 5.96-6.04 (m, 1H) 6.65-6.75 (m, 2H) 6.91 (s, 1H) 7.42 (s, 1H) 7.49-7.57 (m, 2H) 7.57-7.65 (m, 1H) 8.08-8.15 (m, 1H). MS APCI (+/−) 548.2/546.2

### Example 44

### 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)pentan-3-ol

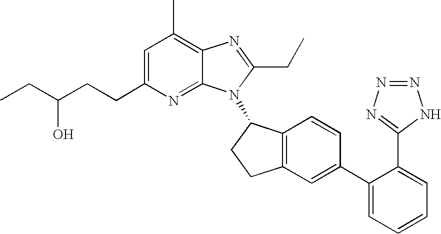

Pent-4-yn-2-ol was used as the alkyne according to the method of Example 24. ¹H NMR (400 MHz, DMSO-d₆) δ ppm 0.97 (dd, J=6.04, 3.70 Hz, 3H), 1.24 (t, J=7.02 Hz, 3H), 1.28-1.39 (m, 2H), 1.49-1.74 (m, 2H), 2.48 (s, 3H), 2.52-2.58 (m, 1H), 2.60-2.85 (m, 5H), 2.93-3.07 (m, 1H), 3.47-3.61 (m, 1H), 4.29 (bs, 1H), 6.34 (bs, 1H), 6.69-6.81 (m, 2H), 6.87 (s, 1H), 7.14 (s, 1H), 7.51-7.61 (m, 2H), 7.61-7.72 (m, 2H); CIMS: 508.2 (APCI)+; 506.2 (APCI)−; HPLC: >99% purity; Rt=10.073 min; method A.

### Example 45

### (R)-1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)pentan-3-ol

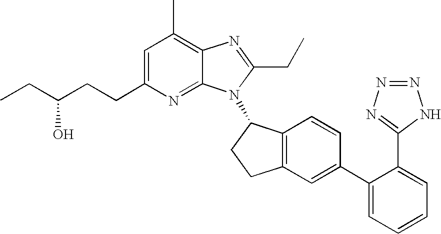

Pent-1-yn-3-ol was used as the alkyne according to the method of Example 24. ¹H NMR (400 MHz, DMSO-d) δ ppm 0.79 (t, J=7.41 Hz, 3H), 1.25 (t, J=7.60 Hz, 3H), 1.28-1.40 (m, 2H), 1.50-1.62 (m, 1H), 1.62-1.75 (m, 1H), 2.48 (s, 3H), 2.53-2.60 (m, 1H), 2.60-2.88 (m, 5H), 2.92-3.07 (m, 1H), 4.35 (bs, 1H), 6.33 (bs, 1H), 6.69-6.79 (m, 2H), 6.87 (s, 1H), 7.14 (s, 1H), 7.50-7.60 (m, 2H), 7.61-7.72 (m, 2H); CIMS: 508.2 (APCI)+; 506.2 (APCI)−; HPLC: 98.20% purity; Rt=10.232 min; method A.

### Example 46

### (S)-1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)pentan-3-ol

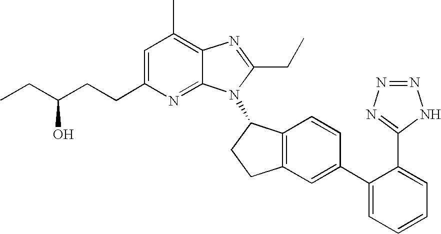

Pent-1-yn-3-ol was used as the alkyne according to the method of Example 24. ¹H NMR (400 MHz, DMSO-d₆) δ ppm 0.80 (t, J=7.41 Hz, 3H), 1.25 (t, =7.80 Hz, 3H), 1.27-1.44 (m, 2H), 1.49-1.63 (m, 1H), 1.63-1.77 (m, 1H), 2.48 (s, 3H), 2.51-2.61 (m, 1H), 2.62-2.89 (m, 5H), 2.93-3.08 (m, 1H), 4.35 (bs, 1H), 6.33 (bs, 1H), 6.68-6.80 (m, 2H), 6.87 (s, 1H), 7.14 (s, 1H), 7.50-7.61 (m, 2H), 7.61-7.72 (m, 2H); CIMS: 508.2 (APCI)+; 506.3 (APCI)−; HPLC: 98.45% purity; Rt=10.581 min; method A.

### Example 47

### 3-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)propan-1-ol

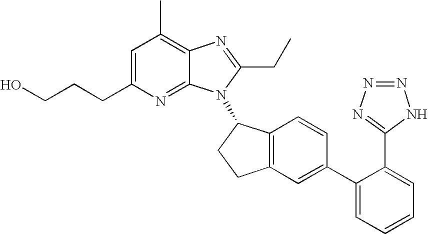

Prop-2-yn-1-ol was used as the alkyne according to the method of Example 24.

### Example 48

### 3-((1S)-5-(2-(1H-tetrazol-5-yl)phenyl)-2,3-dihydro-1H-inden-1-yl)-2-ethyl-5-(3-methoxypropyl)-7-methyl-3H-imidazo[4,5-b]pyridine

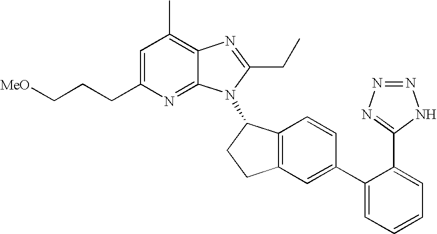

3-Methoxyprop-1-yne was used as the alkyne according to the method of Example 24. ¹H NMR (400 MHz, DMSO-d₆) δ ppm 1.26 (t, J=7.21 Hz, 3H), 1.71-1.85 (m, 2H), 2.47 (s, 3H), 2.51-2.59 (m, 1H), 2.61-2.89 (m, 5H), 2.94-3.06 (m, 1H), 3.16 (s, 3H), 3.20-3.27 (m, 3H), 6.31 (bs, 1H), 6.69-6.80 (m, 2H), 6.86 (s, 1H), 7.13 (s, 1H), 7.50-7.60 (m, 2H), 7.61-7.71 (m, 2H); CIMS: 494.2 (APCI)+; 492.3 (APCI)−; HPLC: 97.52% purity; Rt=10.537 min; method A.

### Example 49

### 4-(3-((1S)-5-(2-(1H-tetrazol-5-yl)phenyl)-2,3-dihydro-1H-inden-1-yl)-7-methyl-2-propyl-3H-imidazo[4,5-b]pyridin-5-yl)butan-2-ol

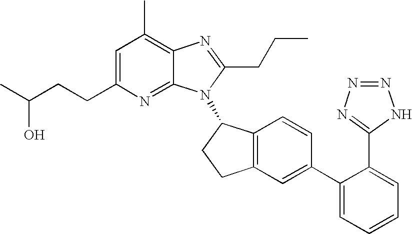

But-3-yn-2-ol was used as the alkyne and coupled with 5-bromo-2-propyl-7-methyl-3-((1S)-5-(2-(1-trityl-1H-tetrazol-5-yl)phenyl)-2,3-dihydro-1H-inden-1-yl)-3H-imidazo[4,5-b]pyridine according to the method of Example 24.

### Example 50

### 1-(3-((1S)-5-(2-(1H-tetrazol-5-yl)phenyl)-2,3-dihydro-1H-inden-1-yl)-7-methyl-2-propyl-3H-imidazo[4,5-b]pyridin-5-yl)-3-methylpentan-3-ol

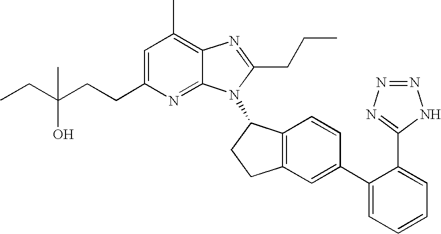

3-Methylpent-1-yn-3-ol was used as the alkyne and coupled with 5-bromo-2-propyl-7-methyl-3-((1S)-5-(2-(1-trityl-1H-tetrazol-5-yl)phenyl)-2,3-dihydro-1H-inden-1-yl)-3H-imidazo[4,5-b]pyridine according to the method of Example 24.

### Example 51

### 4-(3-((1S)-5-(2-(1H-tetrazol-5-yl)phenyl)-2,3-dihydro-1H-inden-1-yl)-7-methyl-2-propyl-3H-imidazo[4,5-b]pyridin-5-yl)-2-phenylbutan-2-ol

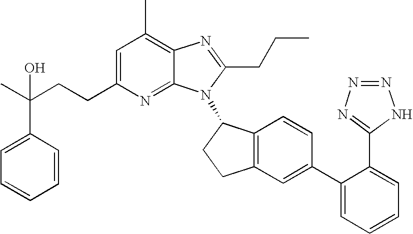

2-Phenylbut-3-yn-2-ol was used as the alkyne and coupled with 5-bromo-2-propyl-7-methyl-3-((1S)-5-(2-(1-trityl-1H-tetrazol-5-yl)phenyl)-2,3-dihydro-1H-inden-1-yl)-3H-imidazo[4,5-b]pyridine according to the method of Example 24. 1H NMR (400 MHz, DMSO-d6) δ ppm 1.31 (br. s., 3H) 1.38 (d, J=9.75 Hz, 3H) 1.96 (br. s., 2H) 2.40 (br. s., 1H) 2.47 (s, 3H) 2.73 (br. s., 3H) 3.03 (br. s., 1H) 6.41 (br. s., 1H) 6.77 (t, J=7.80 Hz, 2H) 6.87 (d, J=7.80 Hz, 2H) 6.93 (br. s., 1H) 6.98 (br. s., 1H) 7.09-7.30 (m, 5H) 7.37 (dd, J=16.77, 7.41 Hz, 2H) 7.45-7.53 (m, 1H) 7.54-7.61 (m, 1H) 7.63-7.71 (m, 2H).

### Example 52

### 1-(2-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)ethyl)cyclohexanol

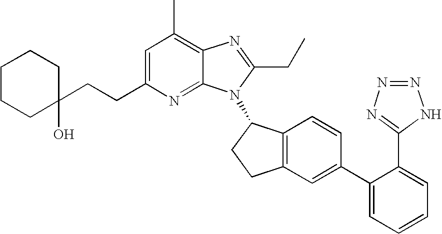

1-Ethynylcyclohexanol was used as the alkyne according to the method of Example 24. 1H NMR (400 MHz, DMSO-d6) δ ppm 1.00-1.44 (m, 8H) 1.54 (d, J=10.53 Hz, 4H) 2.53 (s, 4H) 2.74 (br. s., 2H) 2.94-3.10 (m, 2H) 6.44 (br. s., 1H) 6.79 (d, J=6.24 Hz, 1H) 6.91 (d, J=8.19 Hz, 1H) 7.14 (d, J=8.97 Hz, 2H) 7.46-7.61 (m, 2H) 7.62-7.72 (m, 1H). MS APCI (+/−) 548.2/546.2

### Example 53

### 4-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-2-methylbutan-2-ol

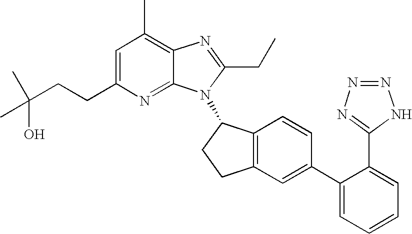

2-Methylbut-3-yn-2-ol was used as the alkyne according to the method of Example 24. ¹H NMR (400 MHz, DMSO-d₆) δ ppm 1.07 (d, J=5.07 Hz, 6H), 1.24 (t, J=6.82 Hz, 3H), 1.61-1.73 (m, 2H), 2.48 (s, 3H), 2.63-2.86 (m, 5H), 2.95-3.07 (m, 1H), 4.20 (bs, 1H), 6.35 (bs, 1H), 6.69-6.80 (m, 2H), 6.87 (s, 1H), 7.14 (s, 1H), 7.52-7.60 (m, 2H), 7.62-7.71 (m, 2H); CIMS: 508.2 (APCI)+; 506.2 (APCI)−; HPLC: 98.49% purity; Rt=10.198 min; method A.

### Example 54

### 3-(2-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)ethyl)-tetrahydrofuran-3-ol

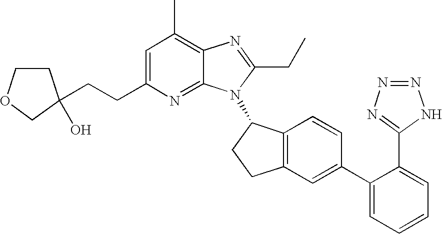

3-Ethynyl-tetrahydrofuran-3-ol was used as the alkyne according to the method of Example 24. ¹H NMR (400 MHz, DMSO-d₆) δ ppm 1.25 (t, J=6.83 Hz, 3H), 1.62-1.78 (m, 2H), 1.78-1.92 (m, 2H), 2.48 (s, 3H), 2.62-2.88 (m, 5H), 2.94-3.07 (m, 1H), 3.41-3.53 (m, 2H), 3.64-3.73 (m, 1H), 3.74-3.84 (m, 1H), 4.66 (bs, 1H), 6.34 (bs, 1H), 6.69-6.80 (m, 2H), 6.89 (s, 1H), 7.14 (s, 1H), 7.51-7.60 (m, 2H), 7.62-7.71 (m, 2H); CIMS: 536.3 (APCI)+; 534.3 (APCI)−; HPLC: 98.65% purity; Rt=9.170 min; method A.

### Example 55

### (R)-1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-3,4-dimethylpentan-3-ol

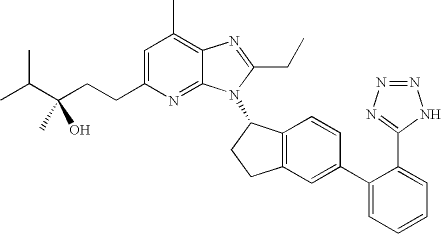

(S)-3,4-Dimethylpent-1-yn-3-ol was used as the alkyne according to the method of Example 24. 1H NMR (400 MHz, DMSO-d6) δ ppm 0.82 (dd, J=18.72, 7.02 Hz, 6H) 0.96 (d, J=7.41 Hz, 3H) 1.27 (t, J=7.02 Hz, 3H) 1.52-1.72 (m, 3H) 2.47 (s, 3H) 2.55-2.87 (m, 4H) 2.90-3.05 (m, 2H) 3.26-3.32 (m, 2H) 4.07-4.18 (m, 1H) 6.27 (br. s., 1H) 6.57-6.65 (m, 1H) 6.75-6.83 (m, 1H) 6.85 (s, 1H) 7.11 (s, 1H) 7.26-7.31 (m, 1H) 7.32-7.38 (m, 2H) 7.47-7.58 (m, 1H).

### Example 56

### 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-3-ethylpentane-3-ol

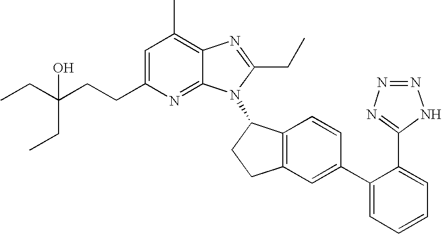

3-Ethylpent-1-yn-3-ol was used as the alkyne according to the method of Example 24. ¹H NMR (400 MHz, DMSO-d₆) δ ppm 0.69-0.81 (m, 6H), 1.26 (t, J=6.64 Hz, 3H), 1.30-1.40 (m, 4H), 1.52-1.64 (m, 2H), 2.48 (s, 3H), 2.49-2.54 (m, 2H), 2.55-2.74 (m, 4H), 2.80 (bs, 1H), 2.93-3.07 (m, 1H), 6.31 (bs, 1H), 6.70-6.80 (m, 2H), 6.86 (s, 1H), 7.12 (s, 1H), 7.50-7.60 (m, 2H), 7.61-7.72 (m, 2H); CIMS: 536.4 (APCI)+, 534.4 (APCI)−; HPLC: >99% purity; Rt=11.324 min; method A.

### Example 57

### (R)-1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-4-methyl pentan-3-ol

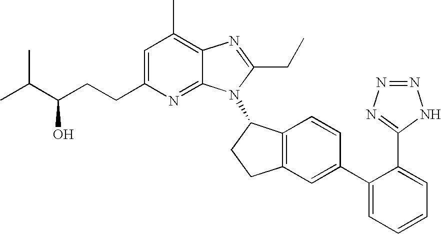

(R)-2-Methylpentan-3-ol was used as the alkyne according to the method of Example 24. ¹H NMR (400 MHz, DMSO-d6) δ ppm 0.71-0.83 (m, 6H) 1.32 (br. s., 3H) 1.42-1.56 (m, 2H) 1.68 (s, 1H) 2.53 (s, 3H) 2.57-2.63 (m, 1H) 2.63-2.78 (m, 2H) 2.84 (br. s., 2H) 2.97-3.08 (m, 2H) 3.09-3.16 (m, 2H) 6.43 (br. s., 1H) 6.73-6.82 (m, 1H) 6.84-6.92 (m, 1H) 7.09 (s, 1H) 7.16 (s, 1H) 7.49-7.61 (m, 2H) 7.62-7.72 (m, 2H).

### Example 58

### 3-((1S)-5-(2-(1H-tetrazol-5-yl)phenyl)-2,3-dihydro-1H-inden-1-yl)-5-(2-cyclohexylethyl)-2-ethyl-7-methyl-3H-imidazo[4,5-b]pyridine

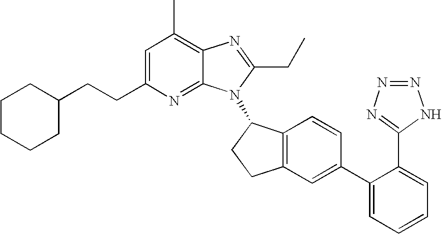

Ethynylcyclohexane was used as the alkyne according to the method of Example 24. ¹H NMR (400 MHz, DMSO-d₆) δ ppm 0.74-0.91 (m, 2H), 1.03-1.19 (m, 4H), 1.26 (t, J=7.41 Hz, 3H), 1.39-1.50 (m, 2H), 1.51-1.74 (m, 5H), 2.47 (s, 3H), 2.51-2.58 (m, 2H), 2.58-2.89 (m, 5H), 2.93-3.06 (m, 1H), 6.31 (bs, 1H), 6.70-6.81 (m, 2H), 6.85 (s, 1H), 7.12 (s, 1H), 7.49-7.60 (m, 2H), 7.61-7.72 (m, 2H); CIMS: 532.3 (APCI)+; 530.3 (APCI)−; HPLC: 95.34% purity; Rt=14.050 min; method A.

### Example 59

### 3-((1S)-5-(2-(1H-tetrazol-5-yl)phenyl)-2,3-dihydro-1H-inden-1-yl)-5-(2-cyclopropylethyl)-2-ethyl-7-methyl-3H-imidazo[4,5-b]pyridine

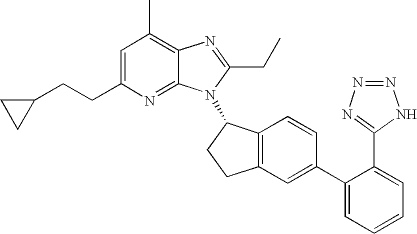

Ethynylcyclopropane was used as the alkyne according to the method of Example 24. CIMS: 490.6 (APCI)+, 488.9 (APCI)−; HPLC: 95.28% purity; Rt=12.173 min, method A.

### Example 60

### 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-3-methylbutan-1-one

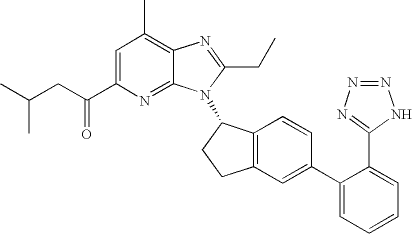

### Step 1. (S)-3-(5-bromo-2,3-dihydro-1H-inden-1-yl)-2-ethyl-7-methyl-3H-imidazo[4,5-b]pyridine-5-carbonitrile

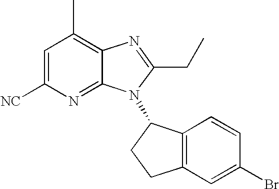

A mixture of the (S)-3-(5-bromo-2,3-dihydro-1H-inden-1-yl)-5-bromo-2-ethyl-7-methyl-3H-imidazo[4,5-b]pyridine (4.3 g, 9.88 mmol), potassium cyanide (0.71 g, 10.9 mmol) and copper(I) cyanide (1.77 g, 19.8 mmol) in DMF (2 mL) was heated at 145 C for 16 h. Ethyl acetate (55 mL) and 1 N HCl (10 mL) were added and the mixture stirred 10 min. The organic layer was separated and the resulting residue was purified on silica gel with 5-25% EtOAc in hexanes as eluant to give (S)-3-(5-bromo-2,3-dihydro-1H-inden-1-yl)-2-ethyl-7-methyl-3H-imidazo[4,5-b]pyridine-5-carbonitrile (1.3 g, 35% yield) as a white solid.

### Step 2

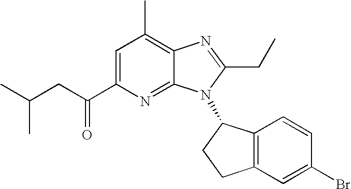

A solution of (S)-3-(5-bromo-2,3-dihydro-1H-inden-1-yl)-2-ethyl-7-methyl-3H-imidazo[4,5-b]pyridine-5-carbonitrile (0.3 g, 0.79 mmol) in toluene (2 mL) was treated with 2 M isobutyl magnesium chloride (1 mL) and the mixture heated at 60 C for 30 min. The reaction was quenched with 2 N HCl (2 mL) and EtOAc (10 mL) and the organic layer separated. The organic residue was purified on a silica gel column using 10-50% EtOAc in hexanes as eluant to give (S)-1-(3-(5-bromo-2,3-dihydro-1H-inden-1-yl)-2-ethyl-7-methyl-3H-imidazo[4,5-b]pyridin-5-yl)-2-methylpropan-1-one (0.23 g, 66% yield) as a light colored solid. 1H NMR (400 MHz, CHLOROFORM-d) δ ppm 0.75-0.94 (m, 6H) 1.40 (br. s., 3H) 2.11 (br. s., 1H) 2.57 (s, 3H) 2.60 (br. s., 1H) 2.80 (br. s., 4H) 2.94 (br. s., 1H) 3.03-3.17 (m, 1H) 3.37 (br. s., 1H) 6.28 (br. s., 1H) 6.77 (d, J=7.80 Hz, 1H) 6.88 (d, J=8.19 Hz, 1H) 7.21 (s, 1H) 7.40 (d, J=7.21 Hz, 1H) 7.45-7.64 (m, 2H) 7.78 (s, 1H) 8.03 (d, J=7.60 Hz, 1H).

### Step 3. 1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)-3-methylbutan-1-one

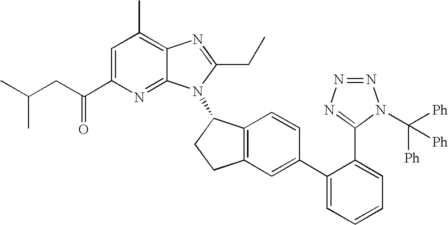

A mixture of (S)-1-(3-(5-bromo-2,3-dihydro-1H-inden-1-yl)-2-ethyl-7-methyl-3H-imidazo[4,5-b]pyridin-5-yl)-3-methylbutan-1-one (230 mg, 0.52 mmol), the tetrazolyl phenyl borate (272 mg, 0.63 mmol), Pd(OAc)₂ (12 mg, 0.052 mmol), triphenylphosphine (55 mg, 0.21 mmol) and potassium carbonate (235 mg, 1.7 mmol) in DME was deoxygenated with bubbling N2 (g) for 10 min. Water (43 mg, 2.4 mmol) was added and N2 (g) bubbled through the mixture for an additional 20 min. The suspension was then heated at 80 C for 16 h. The solution was filtered through celite and purified on a silica gel column using 5-50% EtOAc in hexanes as eluant to give 1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)-3-methylbutan-1-one (320 mg, 82% yield) as a gum.

### Step 4. 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-3-methylbutan-1-one

A solution of 1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)-3-methylbutan-1-one (320 mg, 0.43 mmol) in methanol (20 mL) was refluxed for 4 h. Solvent was removed and the residue purified on a short packed alumina column (neutral, −150 mesh) using 0-10% MeOH in EtOAc as eluant to give 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-3-methylbutan-1-one (167 mg, 77% yield) as a light colored solid.

1H-NMR (CDCl₃)− MS: M+1506.1

### Example 61

### 2-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-3-methylbutan-2-ol

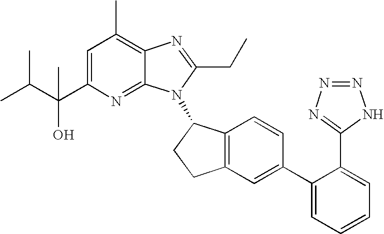

A solution of 1-(2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)-3-methylbutan-1-one (260 mg, 0.35 mmol) in THF (3 mL) was treated with 1.4 M methyl magnesium bromide in THF (0.3 mL) and stirred at room temperature for 2 h. The solvent was removed and the crude product was dissolved in methanol (25 mL) and refluxed for 20 h. Solvent was removed and the residue purified on silica gel using 0-10% MeOH in EtOAc as eluant to give 2-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-3-methylbutan-2-ol (90 mg, 53% yield) as a light colored solid. 1H NMR (400 MHz, CHLOROFORM-d) δ ppm 0.69 (d, J=6.83 Hz, 3H) 0.82 (d, J=6.83 Hz, 3H) 0.93 (d, J=7.22 Hz, 1H) 1.58 (t, J=7.52 Hz, 3H) 1.64 (s, 3H) 1.72-1.85 (m, 1H) 2.25-2.40 (m, 1H) 2.68-2.73 (m, 3H) 2.82-2.96 (m, 1H) 3.25 (d, J=8.79 Hz, 3H) 3.36-3.48 (m, 1H) 4.79 (br. s., 1H) 6.05 (d, J=6.25 Hz, 1H) 6.62 (d, J=7.61 Hz, 1H) 6.91-6.99 (m, 2H) 7.52-7.61 (m, 3H) 7.65 (t, J=7.52 Hz, 1H) 7.91 (d, J=7.61 Hz, 1H). MS: M+1 508 HPLC: HPLC: XTerra RP18 5 uM, 4.6×250 mm column, 90:10 to 10:90, 0.1% TFA water: 0.1% TFA acetonitrile, linear gradient over 20 min at 1.6 mL/min; Retention time=10.2 min.

### Example 62

### 3-((1S)-5-(2-(1H-tetrazol-5-yl)phenyl)-2,3-dihydro-1H-inden-1-yl)-2-ethyl-5-((5-ethyl-1,3,4-oxadiazol-2-yl)methyl)-7-methyl-3H-imidazo[4,5-b]pyridine

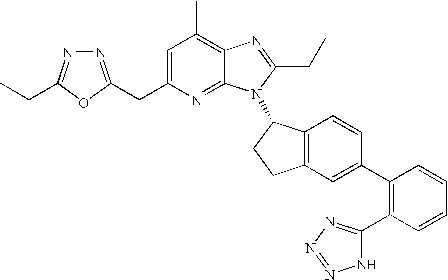

### Step 1: (S)-2-(3-(5-bromo-2,3-dihydro-1H-inden-1-yl)-2-ethyl-7-methyl-3H-imidazo[4,5-b]pyridin-5-yl)acetohydrazide

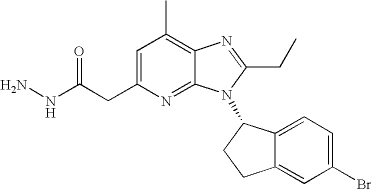

Hydrazine hydrate (0.500 mL, 10.3 mmol) was added to a solution of (S)-[3-(5-Bromo-indan-1-yl)-2-ethyl-7-methyl-3H-imidazo[4,5-b]pyridin-5-yl]-acetic acid methyl ester (0.250 g, 0.584 mmol) in EtOH (25 mL) and heated to reflux for 16 hours. Solvent removed in vacuo and (S)-2-(3-(5-bromo-2,3-dihydro-1H-inden-1-yl)-2-ethyl-7-methyl-3H-imidazo[4,5-b]pyridin-5-yl)acetohydrazide was obtained as a white solid (260 mg 96% yield). Used without further purification. 1H NMR (400 MHz, CHLOROFORM-d) δ ppm 1.42 (t, J=7.42 Hz, 3H) 2.63 (s, 3H) 2.66-2.78 (m, 2H) 2.88 (s, 2H) 3.04-3.18 (m, 1H) 3.30-3.43 (m, 1H) 3.65 (d, J=10.15 Hz, 2H) 3.70 (s, 2H) 6.15 (s, 1H) 6.75 (d, J=7.42 Hz, 1H) 6.89 (s, 1H) 7.30 (dd, J=8.00, 0.98 Hz, 1H) 7.55 (s, 2H). MS: 429.2 (APCI)⁺.

### Step 2: (S)—N′-(2-(3-(5-bromo-2,3-dihydro-1H-inden-1-yl)-2-ethyl-7-methyl-3H-imidazo[4,5-b]pyridin-5-yl)acetyl)propionohydrazide

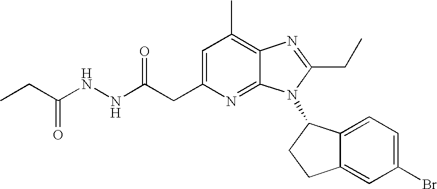

A cooled (0° C.) solution of (S)-2-(3-(5-bromo-2,3-dihydro-1H-inden-1-yl)-2-ethyl-7-methyl-3H-imidazo[4,5-b]pyridin-5-yl)acetohydrazide (0.200 g, 0.630 mmol) in CH₂Cl₂ (3 mL) was treated with DIEA (0.670 mL, 3.76 mmol) and propionyl chloride (0.049 mL, 0.560 mmol) and stirred at 0° C. for 3 hours. Solvent removed in vacuo and residue chromatographed to give (S)—N′-(2-(3-(5-bromo-2,3-dihydro-1H-inden-1-yl)-2-ethyl-7-methyl-3H-imidazo[4,5-b]pyridin-5-yl)acetyl)propionohydrazide (0.219 g, 96% yield). MS: 485.2 (APCI)⁺.

### Step 3: (S)-3-(5-bromo-2,3-dihydro-1H-inden-1-yl)-2-ethyl-5-((5-ethyl-1,3,4-oxadiazol-2-yl)methyl)-7-methyl-3H-imidazo[4,5-b]pyridine

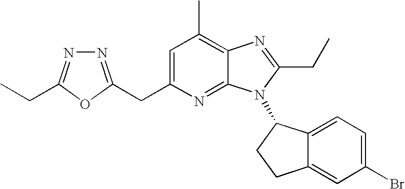

To a solution of (S)—N′-(2-(3-(5-bromo-2,3-dihydro-1H-inden-1-yl)-2-ethyl-7-methyl-3H-imidazo[4,5-b]pyridin-5-yl)acetyl)propionohydrazide (0.219 g, 0.452 mmol) in toluene (15 mL) was added DIEA (0.322 mL, 1.81 mmol) and POCl₃ (0.124 mL, 1.36 mmol) and heated to reflux for 48 hours. Solvent was removed in vacuo and residue chromatographed on silica gel to give of (S)-3-(5-bromo-2,3-dihydro-1H-inden-1-yl)-2-ethyl-5-((5-ethyl-1,3,4-oxadiazol-2-yl)methyl)-7-methyl-3H-imidazo[4,5-b]pyridine (79.3 mg, 37.6% yield) of. MS: 467.1 (APCI)⁺

### Step 4: 2-ethyl-5-((5-ethyl-1,3,4-oxadiazol-2-yl)methyl)-7-methyl-3-((1S)-5-(2-(1-trityl-1H-tetrazol-5-yl)phenyl)-2,3-dihydro-1H-inden-1-yl)-3H-imidazo[4,5-b]pyridine

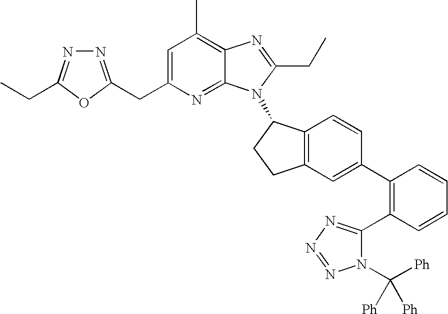

Triphenylphosphine (0.0201 g, 0.0765 mmol), Pd(OAc)₂ (0.00382 g, 0.0170 mmol), potassium carbonate (0.0764 g, 0.552 mmol), (2-(2-trityl-imidazole)-phenyl boronic acid (0.0882 g, 0.204 mmol), (S)-3-(5-bromo-2,3-dihydro-1H-inden-1-yl)-2-ethyl-5-((5-ethyl-1,3,4-oxadiazol-2-yl)methyl)-7-methyl-3H-imidazo[4,5-b]pyridine (0.0793 g, 0.170 mmol) and water (0.0141 mL, 0.782 mmol) were dissolved in DME (10 mL) and degassed for 30 min. The mixture was then heated to 100° C. for 3.5 hours. Reaction was deemed complete by LCMS. Solvent was removed in vacuo and residue chromatographed to give 2-ethyl-5-((5-ethyl-1,3,4-oxadiazol-2-yl)methyl)-7-methyl-3-((1S)-5-(2-(1-trityl-1H-tetrazol-5-yl)phenyl)-2,3-dihydro-1H-inden-1-yl)-3H-imidazo[4,5-b]pyridine (0.095 g, 72% yield) as an off-white foam. MS: 774.3, 532.3 (loss of trityl group) (APCI)⁺. 530.3 (APCI)⁻.

### Step 5: 3-((1S)-5-(2-(1H-tetrazol-5-yl)phenyl)-2,3-dihydro-1H-inden-1-yl)-2-ethyl-5-((5-ethyl-1,3,4-oxadiazol-2-yl)methyl)-7-methyl-3H-imidazo[4,5-b]pyridine

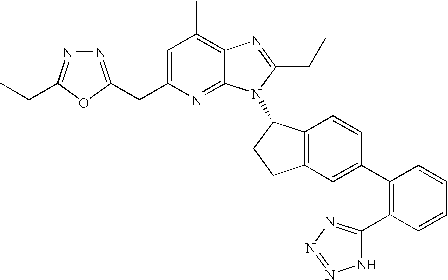

A solution of 2-ethyl-5-((5-ethyl-1,3,4-oxadiazol-2-yl)methyl)-7-methyl-3-((1S)-5-(2-(1-trityl-1H-tetrazol-5-yl)phenyl)-2,3-dihydro-1H-inden-1-yl)-3H-imidazo[4,5-b]pyridine (0.095 g, 0.12 mmol) in MeOH (1 mL) was heated at 80° C. for 16 hours. Solvent was removed in vacuo and the residue chromatographed on silica gel (75% EtOac/hep 1% AcOH) to give 3-((1S)-5-(2-(1H-tetrazol-5-yl)phenyl)-2,3-dihydro-1H-inden-1-yl)-2-ethyl-5-((5-ethyl-1,3,4-oxadiazol-2-yl)methyl)-7-methyl-3H-imidazo[4,5-b]pyridine (0.049 g, 75% isolated yield). 1H NMR (400 MHz, CHLOROFORM-d) δ ppm 1.38 (t, J=7.61 Hz, 3H) 1.46 (t, J=7.61 Hz, 3H) 2.38 (dd, J=12.69, 8.00 Hz, 1H) 2.53-2.62 (m, 1H) 2.64 (s, 3H) 2.75-2.83 (m, 1H) 2.86 (q, J=7.68 Hz, 2H) 2.94-3.03 (m, 1H) 3.07 (q, J=7.55 Hz, 2H) 4.22 (d, J=16.79 Hz, 1H) 4.95 (d, J=16.79 Hz, 1H) 5.87 (t, J=8.20 Hz, 1H) 6.45-6.56 (m, 2H) 6.93 (s, 1H) 7.47-7.56 (m, 3H) 7.57-7.64 (m, 1H) 7.90 (d, J=7.42 Hz, 1H). MS: 532.3 (APCI)⁺.

### Example 63

### 2-((3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)methyl)cyclohexanone

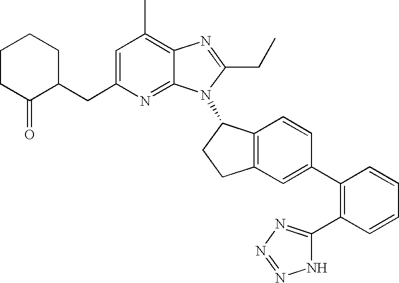

### Steps 1, 2 and 3. (E)-2-((2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)methylene)cyclohexanone

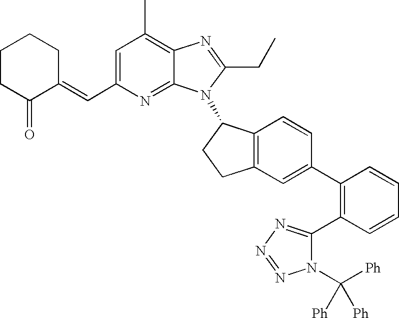

To a mixture of (S)-3-(5-bromo-2,3-dihydro-1H-inden-1-yl)-2-ethyl-7-methyl-3H-imidazo[4,5-b]pyridine-5-carbaldehyde (0.33 g, 0.86 mmol), cyclohexanone (0.10 g, 1.03 mmol), and Mgl₂ in dichloromethane (8 mL) at 23° C. was added diisopropylethylamine (0.19 mL) dropwise. After the addition was complete, the mixture was stirred at 23° C. for 30 min. The reaction mixture was quenched with aqueous NH₄Cl, extracted with dichloromethane (2×50 mL), dried and concentrated. The residue was dissolved in dichloromethane (10 mL) and treated with Et₃N (0.90 mL, 4.80 mmol) and MsCl (0.20 mL, 1.92 mmol) for 2 hours. The resulting mixture was stirred at 23° C. for 16 h, quenched with H₂O, extracted with dichloromethane (2×40 mL), dried and concentrated. The residue was mixed with (2-(2-trityl-imidazole)-phenyl boronic acid (0.22 g, 0.76 mmol), Pd(OAc)₂ (21 mg, 0.09 mmol), PPh₃ (98 mg, 0.38 mmol) and K₂CO₃ (0.35 g, 1.18 mmol) in DME (8 mL) and water (0.08 mL). The mixture was degassed by bubbling N₂ for 5 min and then heated in a sealed vessel at 90° C. for 16 h, cooled to 23° C., concentrated. The residue was purified by chromatography to give (E)-2-((2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)methylene)cyclohexanone (0.26 g, 39% over 3 steps). ¹H NMR (CDCl₃, 400 MHz) δ ppm 7.76 (d, 1H), 7.40-7.00 (m, 12H), 6.98 (s, 2H), 6.80 (m, 7H), 6.50 (d, 1H), 3.00-2.20 (m, 11H), 1.60-1.00 (m, 9H).

### Step 4. 2-((2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)methyl)cyclohexanone

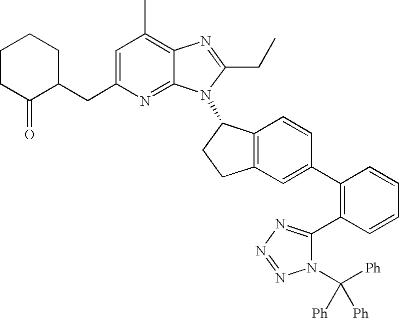

(E)-2-((2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)methylene)cyclohexanone (0.26 g, 0.34 mmol) and 10% Pd—C (0.1 g) in EtOAc (50 mL) was hydrogenated at 50 psi for 2 h, filtered through a pad of celite and concentrated to give 4 (0.26 g, 100%). ¹H NMR (CDCl₃, 400 MHz): δ 7.90 (d, 1H), 7.60-7.20 (m, 16H), 7.10 (s, 1H), 6.95 (m, 4H), 6.85 (2H), 3.35 (m, 1H), 3.15 (m, 1H), 3.00-2.00 (m, 13H), 1.80-1.00 (m, 9H). MS: 774 (M⁺+1)

### Step 5. 2-((3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)methyl)cyclohexanone

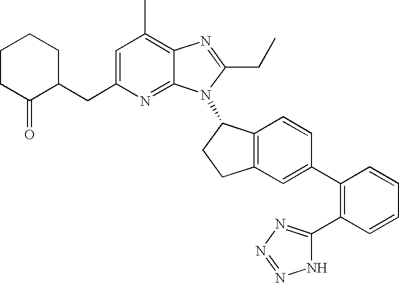

A solution of 2-((2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)methyl)cyclohexanone (80 mg, (0.10 mmo) in MeOH was refluxed for 3 h, cooled to RT and concentrated. The residue was purified by chromatography to give a mixture of (S)-2-((3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)methyl)cyclohexanone and (R)-2-((3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)methyl)cyclohexanone (25 mg). ¹H NMR (CDCl₃, 400 MHz) δ ppm 8.00 (m, 1H), 7.50 (m, 3H), 7.30-7.20 (m, 1H), 7.00-6.50 (m, 3H), 3.40-1.00 (m, 23H). MS: 532.27 (M⁺+1). HPLC: 87.25%.

### Example 64

### 2-((3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)methyl)cyclohexanol

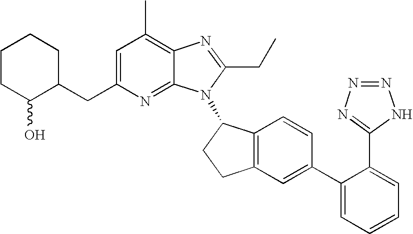

Step 1. A cooled (0° C.) solution of 2-((2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)methyl)cyclohexanone (0.18 g, 0.23 mmol) was treated with lithium aluminum hydride (0.26 mL, 0.26 mmol, 1 M in THF) and stirred at 0° C. for 30 min. The mixture was quenched with aqueous ammonium chloride (0.1 mL) diluted with EtOAc (20 mL), dried over sodium sulfate (5 g), filtered and concentrated. The residue was refluxed in methanol (5 mL) for 3 h, cooled to RT and concentrated. The residue was purified on silica gel to give 2-((3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)methyl)cyclohexanol as two distinct stereoisomers 63a (17 mg) and 63b (20 mg). Absolute stereochemistry was not determined.

Data for 64a: ¹H NMR (CDCl₃, 400 MHz) δ ppm 8.25 (d, 1H), 7.60-7.40 (m, 3H), 7.20 (s, 1H), 7.10 (d, 1H), 6.80 (m, 2H), 6.00 (t, 1H), 5.80 (br s, 1H), 3.30-2.90 (m, 6H), 2.80-2.50 (m, 5H), 1.60-1.00 (m, 13H). MS: 534.23 (M⁺+1). HPLC: 92.98%

Data for 64b: ¹H NMR (CDCl₃, 400 MHz) δ ppm 8.20 (d, 1H), 7.83 (m, 1H), 7.80 (m, 2H), 7.27 (s, 1H), 7.00 (d, 1H), 6.90 (m, 2H), 6.00 (m, 1H), 4.70 (br s, 1H), 3.46 (m, 2H), 3.20 (m, 3H), 2.90 (m, 1H), 2.80-2.60 (m, 5H), 1.90-1.00 (m, 13H) MS: 534.23 (M⁺+1). HPLC: 96.97%

### Example 65

### 2-((3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)methyl)cyclopentanol

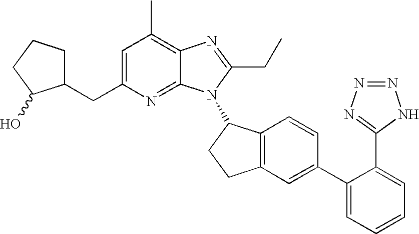

### Step 1: (E)-2-((2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)methylene)cyclopentanone

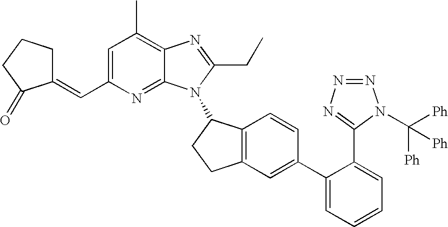

To a mixture of (S)-3-(5-bromo-2,3-dihydro-1H-inden-1-yl)-2-ethyl-7-methyl-3H-imidazo[4,5-b]pyridine-5-carboxaldehyde (0.34 g, 0.89 mmol), cyclopentanone (90 mg, 1.07 mmol), magnesium iodide (0.30 g, 1.07 mmol) in dichloromethane (8 mL) was added diisopropylethylamine (0.20 mL) dropwise. After the addition was over, the mixture was stirred at RT for 30 min. The reaction mixture was quenched with aqueous NH₄Cl, extracted with dichloromethane (50 mL), dried and concentrated. The residue was dissolved in dichloromethane (10 mL) and treated with Et₃N (0.63 mL, 4.50 mmol) and MsCl (0.14 mL, 1.78 mmol). The resulting mixture was stirred at RT for 16 h, quenched with H₂O, extracted with dichloromethane (40 mL), dried and concentrated. The residue was mixed with the tetrazolyl phenyl borate adduct (0.14 g, 0.32 mmol), Pd(OAc)₂ (9 mg, 0.04 mmol), PPh₃ (42 mg, 0.16 mmol) and KCO₃ (70 mg, 0.50 mmol) in DME (3 mL) and water (2 drops). The mixture was degassed by bubbling N₂ for 5 min and then heated in a sealed vessel at 90° C. for 16 h, cooled to RT, concentrated. The residue was purified on silica gel to give (E)-2-((2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)methylene)cyclopentanone (0.11 g, 16% over 3 steps). ¹H NMR (CDCl₃, 400 MHz) δ ppm 7.95 (d, 1H), 7.60-7.20 (m, 14H), 7.18 (m, 2H), 7.00 (m, 6H), 6.66 (d, 1H), 3.10 (m, 1H), 3.00-2.40 (m, 8H), 2.30 (m, 2H), 1.80 (m, 2H), 1.40 (m, 3H), 1.23 (m, 2H).

### Step 2

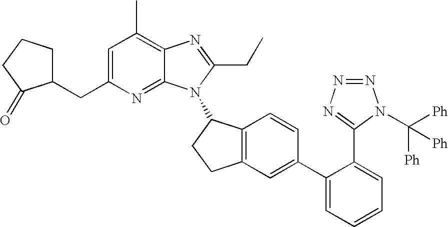

A mixture of (E)-2-((2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)methylene)cyclopentanone (0.11 g, 0.14 mmol) and 10% Pd—C (0.1 g) in EtOAc (50 mL) was hydrogenated at 50 psi for 2 h, filtered through a pad of celite and concentrated to give 2-((2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)methyl)cyclopentanone (0.11 g, 100%). MS: 760 (M⁺+1)

### Steps 3 and 4: (R)- and (S)-2-((3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)methyl)cyclopentanol

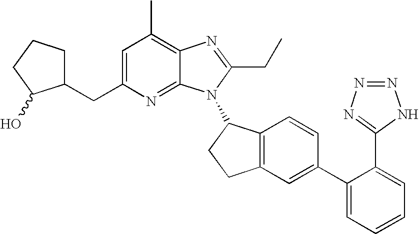

To a cooled (0° C.) solution of 2-((2-ethyl-7-methyl-3-((1S)-5-(2-(1-trityl-1H-tetrazol-5-yl)phenyl)-2,3-dihydro-1H-inden-1-yl)-3H-imidazo[4,5-b]pyridin-5-yl)methyl)cyclopentanone (0.11 g, 0.14 mmol) in THF (3 mL) was added lithium aluminum hydride (0.22 mL, 0.22 mmol). The mixture was stirred at 0° C. for 30 min, quenched with aqueous ammonium chloride (0.1 mL) diluted with EtOAc (20 mL), dried, and concentrated. The residue was dissolved in methanol (4 mL) and refluxed for 3 h, cooled to RT and concentrated. The residue was purified by chromatography to give Example 65 as two stereoisomers of 2-((3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)methyl)cyclopentanol 65a (16 mg) and 6b (22 mg). Absolute stereochemistry was not determined.

Data for 65a: ¹H NMR (CDCl₃, 400 MHz) δ ppm 8.20 (m, 1H), 7.60 (m, 3H), 7.30-7.20 (m, 1H), 7.00-6.60 (m, 3H), 3.40-1.00 (m, 22H). MS: 520.12 (M⁺+1). HPLC: 93.11%.

Data for 65b: ¹H NMR (CDCl₃, 400 MHz) δ ppm 8.20 (m, 1H), 7.60 (m, 4H), 6.90-6.70 (m, 3H), 5.97 (m, 1H), 4.00-1.00 (m, 22H). MS: 520.25 (M⁺+1). HPLC: 95.41%.

Examples 66 and 67 were prepared by an analogous procedure as Example 4 except that the appropriate heterocyclic anion was used as a nucleophile to displace the mesylate.

### Example 66

### 3-((1S)-5-(2-(1H-tetrazol-5-yl)phenyl)-2,3-dihydro-1H-inden-1-yl)-5-((2H-1,2,3-triazol-2-yl)methyl)-2-ethyl-7-methyl-3H-imidazo[4,5-b]pyridine

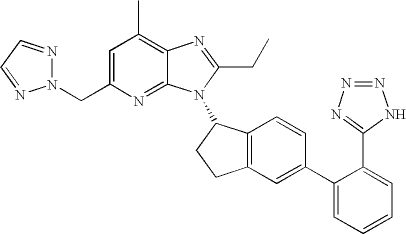

The sym-triazole anion was used to displace the mesylate. 1H NMR (400 MHz, CHLOROFORM-d) δ ppm 1.46 (t, J=7.42 Hz, 3H) 2.10 (s, 3H) 2.31-2.44 (m, 1H) 2.50-2.62 (m, 1H) 2.66 (s, 3H) 2.81-2.91 (m, 1H) 2.92-3.04 (m, 1H) 3.04-3.19 (m, J=6.64 Hz, 2H) 5.70 (d, J=15.62 Hz, 1H) 5.91 (s, 1H) 6.20 (d, J=15.62 Hz, 1H) 6.58 (d, 1H) 6.63 (d, 1H) 7.01 (s, 1H) 7.34 (s, 1H) 7.52-7.58 (m, 2H) 7.59 (s, 2H) 7.62-7.69 (m, 1H) 8.00 (d, J=7.81 Hz, 1H).

### Example 67

### 3-((1S)-5-(2-(1H-tetrazol-5-yl)phenyl)-2,3-dihydro-1H-inden-1-yl)-5-((3,5-dimethyl-1H-pyrazol-1-yl)methyl)-2-ethyl-7-methyl-3H-imidazo[4,5-b]pyridine

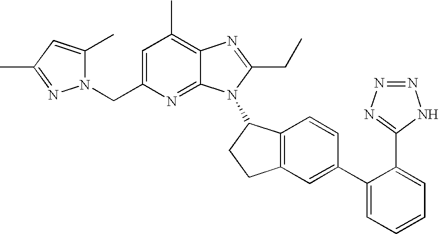

3,5-Dimethyl-1H-pyrazole anion was used to displace the mesylate. 1H NMR (400 MHz, CHLOROFORM-d) δ ppm 1.90 (d, J=19.88 Hz, 3H) 1.99 (d, J=24.17 Hz, 3H) 2.47 (s, 2H) 2.76 (br. s., 1H) 2.85 (br. s., 3H) 3.05 (br. s., 2H) 3.49 (s, 2H) 5.24 (d, J=18.72 Hz, 1H) 5.88 (d, J=17.55 Hz, 1H) 5.92 (br. s., 1H) 5.99 (s, 1H) 6.40-6.58 (m, 2H) 7.30 (br. s., 1H) 7.48-7.60 (m, 2H) 7.62 (d, J=7.41 Hz, 1H) 7.84 (d, J=7.41 Hz, 1H).

### Example 68

### 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-3,4,4-trimethylpentan-3-ol

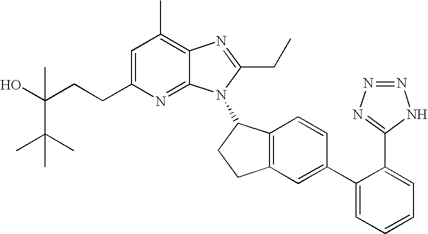

3,4,4-Trimethylpent-1-yn-3-ol was used as the alkyne according to the method of Example 24. 1H NMR (400 MHz, DMSO-d6) δ ppm 0.81 (d, J=7.03 Hz, 9H) 0.99 (s, 3H) 1.34 (br. s., 3H) 1.48-1.61 (m, 1H) 1.65-1.80 (m, 1H) 2.55 (s, 1H) 2.74 (br. s., 2H) 2.83 (br. s., 1H) 2.96-3.09 (m, 2H) 6.45 (br. s., 1H) 6.76-6.85 (m, 1H) 6.91-6.98 (m, 1H) 7.10-7.15 (m, 1H) 7.18 (s, 1H) 7.51 (dd, J=7.42, 3.90 Hz, 1H) 7.57 (t, J=7.03 Hz, 1H) 7.62-7.71 (m, 2H).

### Example 69

### 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-3,5-dimethylhexan-3-ol

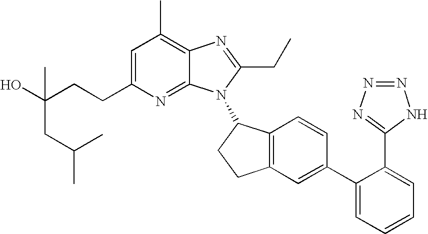

3,5-Dimethylhex-1-yn-3-ol was used as the alkyne according to the method of Example 24. 1H NMR (400 MHz, DMSO-d6) δ ppm 0.84-0.93 (m, 6H) 1.05 (d, J=8.98 Hz, 3H) 1.18-1.34 (m, 5H) 1.66 (br. s., 2H) 1.69-1.79 (m, 1H) 2.47 (s, 3H) 2.67 (br. s., 4H) 2.75-2.88 (m, 1H) 2.91-3.04 (m, 1H) 6.27 (br. s., 1H) 6.58-6.65 (m, 1H) 6.74-6.82 (m, 1H) 6.85 (s, 1H) 7.06-7.16 (m, 1H) 7.27-7.42 (m, 3H) 7.49-7.58 (m, 1H).

What is claimed:

1. A compound of Formula I: 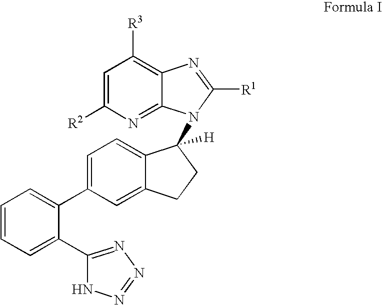
wherein:
R¹ is (C₁-C₄)alkyl or ethoxy;
R² is (C₁-C₈)alkyl, (C₂-C₈)alkenyl or (C₂-C₈)alkynyl, said (C₁-C₈)alkyl, (C₂-C₈)alkenyl or (C₂-C₈)alkynyl mono-, di- or tri-substituted independently with hydroxyl, (C₁-C₅)alkylcarbonyloxy, benzylcarbonyloxy, (C₁-C₃)alkoxy, halo, trifluoromethyl, nitrile, oxo or a 3 to 8 membered partially saturated, fully saturated or fully unsaturated ring optionally having one to three heteroatoms selected independently from one, two or three N, one O or one S and said 3 to 8 membered ring optionally mono-, di- or tri-substituted independently with halo, (C₂-C₆)alkenyl, (C₁-C₆)alkyl, hydroxyl, (C₁-C₆)alkoxy, (C₁-C₄)alkylthio, amino, nitro, cyano, oxo, carboxy, (C₁-C₆)alkyloxycarbonyl or mono-N— or di-N,N—(C₁-C₆)alkylamino wherein said (C₁-C₆)alkyl substituent is optionally mono-, di- or tri-substituted independently with halo, hydroxyl, (C₁-C₆)alkoxy, (C₁-C₄)alkylthio, amino, nitro, cyano, oxo, carboxy, (C₁-C₆)alkyloxycarbonyl or mono-N— or di-N,N—(C₁-C₆)alkylamino and wherein said (C₁-C₆)alkyl substituent is also optionally substituted with from one to nine fluorines; and
R³ is CH₃, or
a pharmaceutically acceptable salt thereof.

2. A compound as recited in claim 1 wherein:
R¹ is (C₂-C₄) alkyl; and
R² is (C₁-C₈)alkyl, said (C₁-C₈)alkyl mono- or di-substituted independently with hydroxyl, (C₁-C₅)alkylcarbonyloxy, benzylcarbonyloxy, (C₁-C₃)alkoxy, halo, keto or a 5 to 6 membered partially saturated, fully saturated or unsaturated ring optionally having one or two N, and said 5 to 6 membered ring optionally mono-, di- or tri-substituted independently with hydroxy, halo, (C₁-C₃)alkoxy, (C₁-C₄)alkyl or oxo; or
a pharmaceutically acceptable salt thereof.

3. A compound as recited in claim 2 wherein
R² is (C₂-C₅)alkyl, said (C₂-C₅)alkyl mono-substituted with hydroxyl or (C₁-C₅)alkylcarbonyloxy, benzylcarbonyloxy,
or a pharmaceutically acceptable salt thereof.

4. A compound as recited in claim 2 wherein:
R² is selected from (C₂-C₄)alkyl, said (C₂-C₄)alkyl mono-substituted with (C₁-C₃)alkoxy,
or a pharmaceutically acceptable salt thereof.

5. A compound as recited in claim 2 wherein:
R² is selected from (C₂-C₅)alkyl, said (C₂-C₅)alkyl mono-substituted with a 5 to 6 membered partially saturated, fully saturated or unsaturated ring optionally having one or two N, and said 5 to 6 membered ring optionally mono-, di- or tri-substituted independently with hydroxyl, halo, (C₁-C₃)alkoxy, (C₁-C₄)alkyl or oxo; or
a pharmaceutically acceptable salt thereof.

6. A compound as recited in claim 2 wherein:
R² is selected from (C₂-C₅)alkyl, said (C₂-C₅)alkyl mono-substituted with hydroxyl, (C₁-C₅)alkylcarbonyloxy, benzylcarbonyloxy, or (C₁-C₃)alkoxy, and mono-substituted with a 5 to 6 membered partially saturated, fully saturated or fully unsaturated ring optionally having one or two N, and said 5 to 6 membered ring optionally mono-, di- or tri-substituted independently with hydroxy, halo, (C₁-C₃)alkoxy, (C₁-C₄)alkyl or oxo; or
a pharmaceutically acceptable salt thereof.

7. A compound as recited in claim 3 wherein
R¹ is ethyl;
R² is (C₂-C₅)alkyl, said (C₂-C₅)alkyl mono-substituted with hydroxyl or (C₁-C₅)alkylcarbonyloxy, benzylcarbonyloxy,
or a pharmaceutically acceptable salt thereof.

8. A compound as recited in claim 4 wherein
R¹ is ethyl;
R² is selected from (C₂-C₄)alkyl, said (C₂-C₄)alkyl mono-substituted with (C₁-C₃)alkoxy.

9. A compound which is
a. (S)-1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-2-methylpropan-1-ol);
b. 3-((1S)-5-(2-(1H-tetrazol-5-yl)phenyl)-2,3-dihydro-1H-inden-1-yl)-2-ethyl-5-(2-methoxyethyl)-7-methyl-3H-imidazo[4,5-b]pyridine;
c. 1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)-2-methylpropan-2-ol;
d. 2-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)ethanol; or
e. 2-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)ethyl acetate
or a pharmaceutically acceptable salt thereof.

10. A compound having the structure

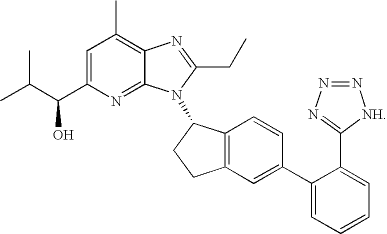

11. A compound having the structure

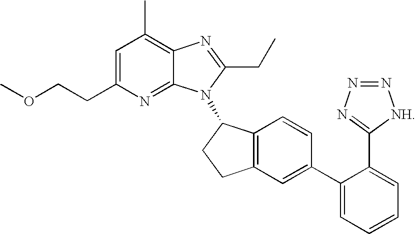

12. A compound having the structure

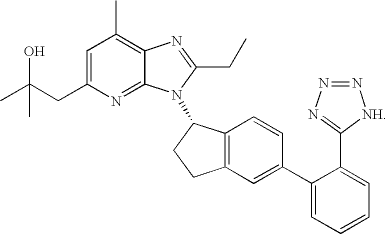

13. A compound having the structure

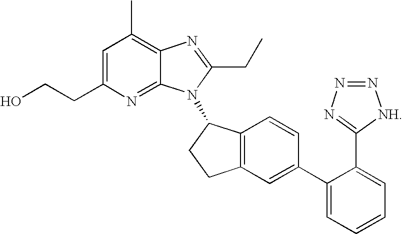

14. A compound having the structure

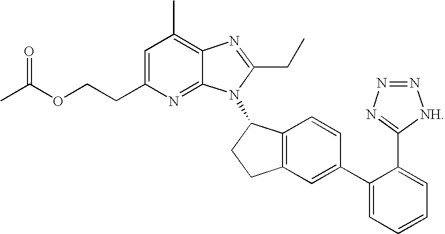

15. A compound of Formula IIA: 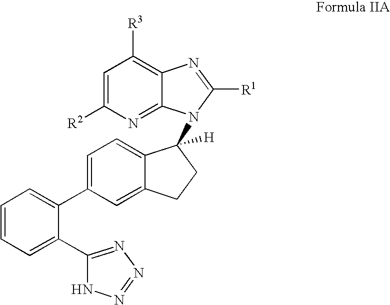
wherein:
R¹ is selected from ethyl, n-propyl, iso-propyl, cyclopropyl, n-butyl, s-butyl, isobutyl, and t-butyl;
R² is n-butyl substituted by 1 or 2 groups selected from OH, C₁-C₃ alkoxy, C(O)OR^{a} or C(O)NR^{a}R^{b} and C₃-C₆ cycloalkyl;
R^{a} is selected from H, C₁-C₆ alkyl, —(CH₂)₀₋₃—(C₃-C₇ cycloalkyl), phenyl and benzyl;
R^{b} is selected from H and C₁-C₆ alkyl; and
R³ is selected from CH₃; or
a pharmaceutically acceptable salt thereof.

16. A compound of Formula IIIA: 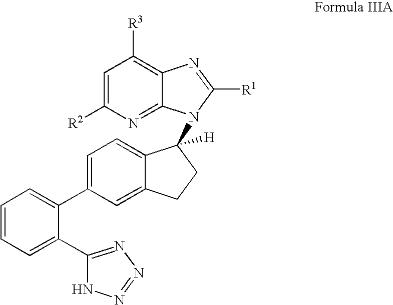
wherein:
R¹ is selected from ethyl, n-propyl iso-propyl, cyclopropyl, n-butyl, s-butyl, isobutyl, and t-butyl;
R² is isobutyl substituted by 1 or 2 groups selected from OH, C₁-C₃ alkoxy, C(O)OR^{a} or C(O)NR^{a}R^{b} and C₃-C₆ cycloalkyl;
R^{a} is selected from H, C₁-C₆ alkyl, —(CH₂)₀₋₃-(C₃-C₇ cycloalkyl), phenyl and benzyl;
R^{b} is selected from H and C₁-C₆ alkyl; and
R³ is CH₃;
or a pharmaceutically acceptable salt thereof.

17. A compound as recited in claim 1 selected from the group of
(S,S)-4-(2-Ethyl-7-methyl-3-{5-[2-(1H-tetrazol-5-yl)-phenyl]-indan-1-yl}-3H-imidazo[4,5-b]pyridin-5-yl)-butan-2-ol;
(S)-1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)propan-2-ol
(R)-1-(3-((1S)-5-(2-(1H-tetrazol-5-yl)phenyl)-2,3-dihydro-1H-inden-1-yl)-2-ethyl-7-methyl-3H-imidazo[4,5-b]pyridin-5-yl)propan-2-ol
(S)-1-(2-Ethyl-7-methyl-3-{(S)-5-[2-(1H-tetrazol-5-yl)-phenyl]-indan-1-yl}-3H-imidazo[4,5-b]pyridin-5-yl)-2-methyl-propan-1-ol;
3-((1S)-5-(2-(1H-tetrazol-5-yl)phenyl)-2,3-dihydro-1H-inden-1-yl)-2-ethyl-5-(2-methoxyethyl)-7-methyl-3H-imidazo[4,5-b]pyridine;
1-((S)-2-Ethyl-7-methyl-3-{5-[2-(1H-tetrazol-5-yl)-phenyl]-indan-1-yl}-3H-imidazo[4,5-]pyridin-5-yl)-2-methyl-propan-2-ol;
3-((1S)-5-(2-(1H-tetrazol-5-yl)phenyl)-2,3-dihydro-1H-inden-1-yl)-2-ethyl-5-(2-methoxypropan-2-yl)-7-methyl-3H-imidazo[4,5-b]pyridine;
3-((1S)-5-(2-(1H-tetrazol-5-yl)phenyl)-2,3-dihydro-1H-inden-1-yl)-2-ethyl-7-methyl-5-(pyridin-2-ylmethyl)-3H-imidazo[4,5-b]pyridine;
(S)-2-Ethyl-7-methyl-5-(2-pyridin-3-yl-ethyl)-3-{5-[2-(1H-tetrazol-5-yl)-phenyl]-indan-1-yl}-3H-imidazo[4,5-b]pyridine;
(S)-2-ethyl-(5-ethyl-[1,3,4]oxadiazol-2-ylmethyl)-7-methyl-3-{5-[2-(1H-tetrazol-5-yl)-phenyl]-indan-1-yl}-3H-imidazo[4,5-b]pyridine;
(S)-(2-Ethyl-7-methyl-3-{5-[2-(1H-tetrazol-5-yl)-phenyl]-indan-1-(S)-yl}-3H-imidazo[4,5-b]pyridin-5-yl)-(S)-phenyl-methanol;
(S)-(2-Ethyl-7-methyl-3-{5-[2-(1H-tetrazol-5-yl)-phenyl]-indan-1-(S)-yl}-3H-imidazo[4,5-b]pyridin-5-yl)-(R)-phenyl-methanol;
2-(S)-(2-Ethyl-7-methyl-3-{5-[2-(1H-tetrazol-5-yl)-phenyl]-indan-1-(S)-yl}-3H-imidazo[4,5-b]pyridin-5-ylmethyl)-cyclohexanone; and
2-(R)-(2-Ethyl-7-methyl-3-{5-[2-(1H-tetrazol-5-yl)-phenyl]-indan-1-(S)-yl}-3H-imidazo[4,5-b]pyridin-5-ylmethyl)-cyclohexanone;
or a pharmaceutically acceptable salt form thereof.

18. A pharmaceutical composition comprising a pharmaceutically effective amount of a compound of claim 1, or a pharmaceutically acceptable salt thereof, and a pharmaceutically acceptable carrier or excipient.

19. A method of treatment of a disease selected from the group of type 2 diabetes, insulin resistance, hyperinsulinemia, hyperlipidemia, hypertriglyceridemia, metabolic syndrome, congestive heart failure, and hypertension in a mammal in need of such treatment, the method comprising administering to the mammal a pharmaceutically effective amount of a compound of claim 1, or a pharmaceutically acceptable salt thereof.
